(19) United States
(12) Patent Application Publication
(10) Pub. No.: US 2005/0226879 A1
(43) Pub. Date: Oct. 13, 2005

(54) Methods and compositions related to inhibiting nuclear envelope breakdown

(76) Inventors: Katharine S Ulmann, Salt Lake City, UT (US); Jin Liu, Davis, CA (US); Amy Prunuske, Salt Lake City, UT (US); Christian Dimaano, Salt Lake City, UT (US)

Correspondence Address: NEEDLE & ROSENBERG, P.C., SUITE 1000, 999 PEACHTREE STREET, ATLANTA, GA, 30309-3915, US

(21) Appl. No.: 10/528,183

(22) PCT Filed: Sep. 17, 2003
(86) PCT No.: PCT/US03/29267

§ 371 Date: May 27, 2005

Related U.S. Application Data
(60) Provisional application No. 60/411,248, filed on Sep. 17, 2002.

(51) Int. Cl. A61K 39/395; C12Q 1/68; A61K 31/19

(52) U.S. Cl. 424/146.1; 514/575; 435/6

(57) ABSTRACT

Disclosed are compositions and methods for inhibiting nuclear envelope breakdown.

### I. SUMMARY

In accordance with the purposes of this invention, as embodied and broadly described herein, this invention, in one aspect, relates to compositions and methods relating to inhibition of nuclear envelope breakdown.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### II. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 is a schematic illustration of the use of extract from Xenopus eggs, which provide a rich source of cellular material.

FIG. 2 is a schematic illustration of initial steps in a system for recapitulating nuclear structure and function in vitro.

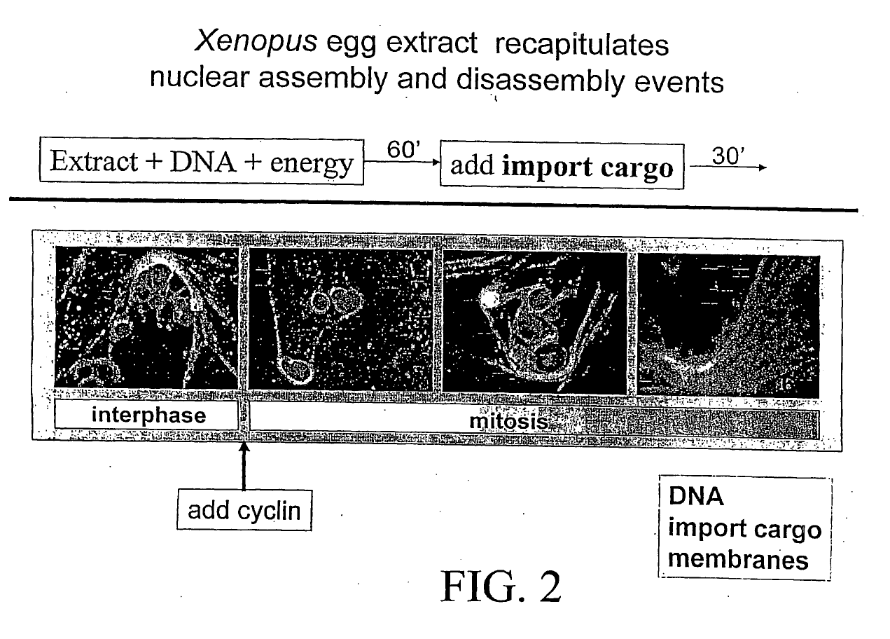

FIG. 3 contains illustrations of the Nup153 nuclear pore protein determined to have a role in nuclear disassembly and a domain-specific recombinant protein containing the central zinc-finger region of Nup153.

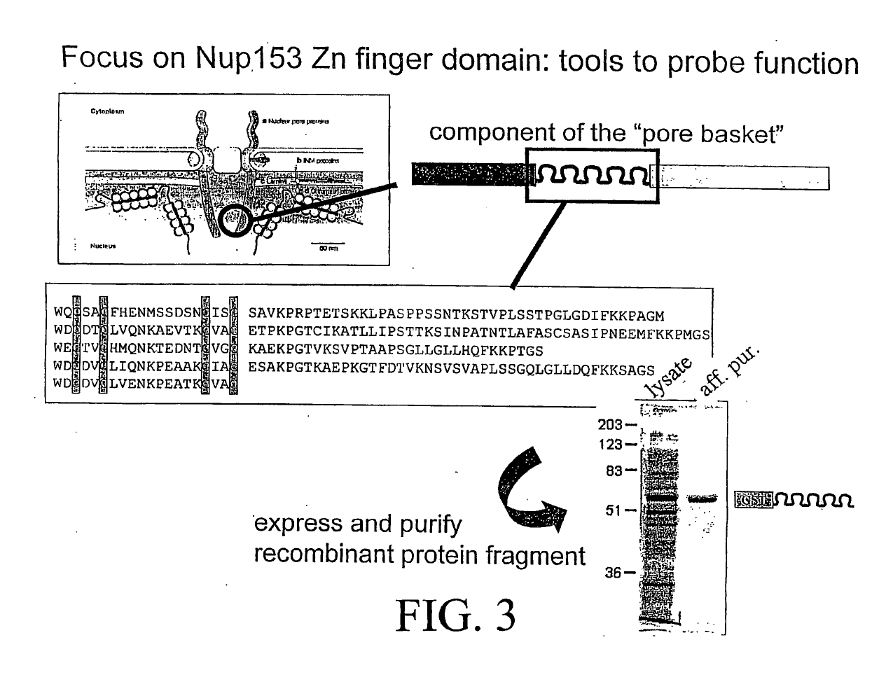

FIG. 4 illustrates the failure of nuclear envelope breakdown in the presence of the recombinant protein with the Nup153 zinc finger domain of FIG. 3.

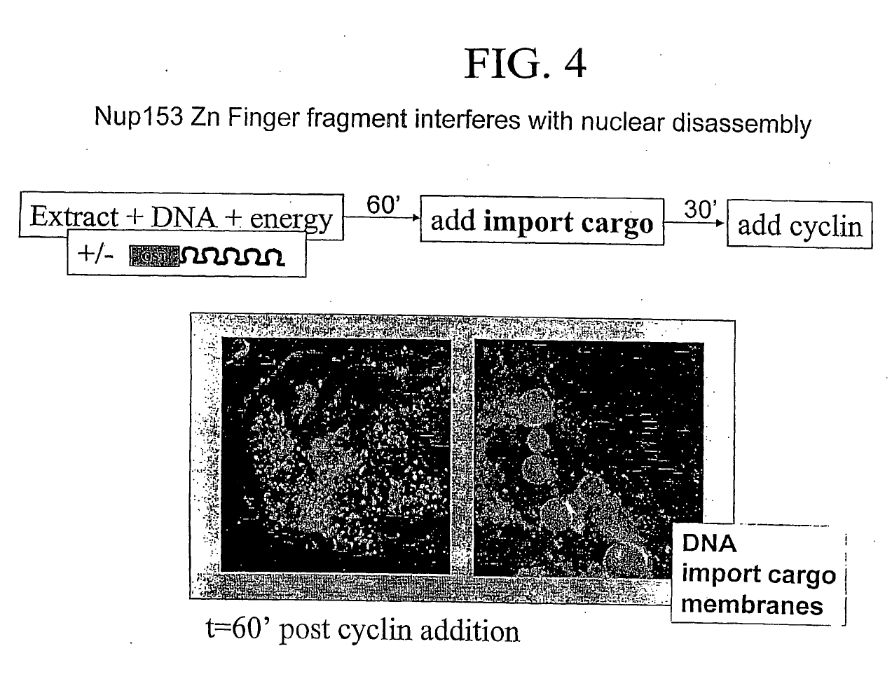

FIG. 5 shows blots of Nup153 specific antibodies.

FIG. 6 illustrates the failure of nuclear envelope breakdown in the presence of the Nup153-specific antibodies of FIG. 5.

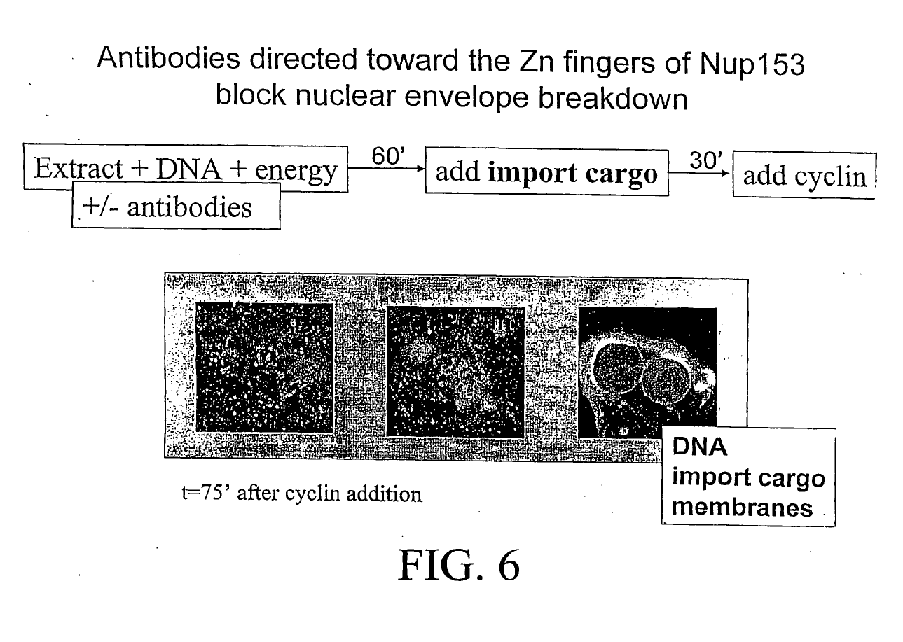

FIG. 7 shows a specific domain of Nup153 exerts a dominant negative effect on nuclear envelope breakdown. FIG. 7A shows a schematic diagram of Xenopus Nup153. “N” and “Z” indicate Nup153-N (amino acids 436-655) and Nup153-Z (amino acids 655-926) fragments used in this study. FIG. 7B shows a coomassie blue staining of purified recombinant proteins GST, GST-N and GST-Z; lanes 1-3. Immunoblot of recombinant proteins before (lanes 4-7) and after (lanes 8-11) nuclei assembly/disassembly assay. Proteins were detected with antibodies directed against GST. Molecular weight markers indicated correspond to 93, 49, 35 and 29 kD. FIG. 7C shows buffer (ELB) or recombinant proteins (3 μg) were added 15 minutes prior to the beginning of the assembly/disassembly assay. The interphase sample was taken after 90 minutes of assembly, immediately prior to cyclin addition. The mitotic sample was taken 75 minutes after cyclin addition. DNA (Hoechst 33258 stain) is shown in blue and merged with the signal for NLS import substrate, shown in red panels a, c, e, g, i, k, m, and o). Membrane staining is shown in panels b, d, f, h, j, l, n and p. Numbers to the right of the panel indicate the percentage of intact nuclei at the 75 minute post-cyclin time point. The bar in panel o indicates 50 μm.

FIG. 8 shows antibodies specific to Nup153 interfere with nuclear envelope breakdown. FIG. 8A shows an immunoblot of fractionated egg extract probed with pre-immune antibody (lane 1), antibody against Nup153-Z (lane 2), antibody against Nup153-N (lane 3) and mAb414 (lane 4). mAb414 reactivity confirms the presence of Nup358, Nup214, Nup153, and Nup62. For both A and B, molecular weight markers indicated are 198, 115 and 93 kD. FIG. 8B shows an immunoprecipitation of egg extract with pre-immune (lane 1), anti-Nup153-Z (lane 2), and anti-Nup153-N (lane 3). Lane 4 was loaded with the equivalent of ˜75% input. The blot was probed with mAb414. C) Pre-immune (PI) or specific antibodies (2.5 μg) or buffer (ELB) were added 15 minutes prior to the beginning of the assembly/disassembly assay. Samples were processed as described in FIG. 7. Nuclear import cargo and DNA are shown; the bar in panel f indicates 50 μm.

FIG. 9 shows the zinc finger domain of Nup153 associates with the COPI complex. FIG. 9A shows a silver staining of gel from GST pulldown assay. 2% of input (XEE, Xenopus egg extract) was loaded in lane 1. Molecular markers indicated are 198, 115, and 93 kD. (a) and (b) indicate two bands subjected to peptide sequencing. FIG. 9B shows sequences of peptides obtained from band (a) and band (b). All sequences are aligned with those of homologous human proteins in the database. FIG. 9C shows an immunoblot of GST pulldown samples with antibodies against human α-COP, β-COP and β′-COP, respectively.

FIG. 10 shows that Anti-β-COP inhibits nuclear envelope breakdown. A) Immunoblot of egg extract probed with pre-immune (lane 1) and anti-Xenopus-β-COP peptide antibody (lane 2). In lane 3, anti-β-COP (0.2 μg/ml) was incubated with β-COP peptide (0.2 μg/ml) for 15 min at room temperature before adding to the blot. In both A and B, molecular weight markers indicated are 115 and 93 kD. FIG. 10B shows an immunoprecipitation of egg extract with pre-immune antibody (lane 1) and anti-Xenopus β-COP peptide antibody (lane 2). In lane 3, 2% of input egg extract was loaded. The blot was probed with anti-Xenopus-β-COP peptide antibody. * indicates IgG. FIG. 10C shows bBuffer (ELB), antibodies (5 μg) and β-COP peptide (5 μg) were added 15 minutes prior to the assembly/disassembly assay. In “PI+peptide” and “anti-β-COP+peptide” groups, antibodies were incubated with the peptide for 15 min at room temperature before added to crude egg extract. Samples were processed as described in FIG. 7. Nuclear import cargo and DNA are shown; the bar in panel 1 indicates 50 μM.

FIG. 11 shows that β-COP is recruited to the nuclear envelope during nuclear disassembly. FIG. 11A shows an indirect immunofluorescence was performed on HeLa cells following cell cycle synchronization. Cells in early prophase (indicated by arrows) show nuclear accumulation of cyclin B (panel b). The localization of β-COP is shown in panel a and Hoechst detection of DNA is shown in panel c. FIG. 11B shows that under the same conditions described above, both β-COP (panel a) and nuclear pore protein reactive with mAb414 (panel b) were detected. The merge of the two images is seen in panel c and arrows highlight cells exhibiting colocalization. The size bar in panel c indicates 50 μm for panels in both A and B. FIG. 11C shows that HeLa cells treated as above were examined by confocal microscopy for β-COP (green) and nuclear pore proteins (mAb414, red). The size bar in panel b indicates 25 μm. FIG. 11D shows the localization mAb414-reactive nucleoporins (panels e-h) and β-COP (panels a-d) during in vitro nuclear disassembly. Time is either immediately prior to cyclin addition (panels a, e), or 20 minutes (panels b, f) 40 minutes panels c, g), and 60 minutes (panels d, h) after cyclin addition. FIG. 11E shows a recombinant zinc finger region (3 μg; 1.5 μM final concentration) was added 15 minutes prior to the assembly/disassembly assay. The same volume of buffer (ELB) was added to controls. Samples were examined 60 minutes after the addition of cyclin. Samples in panels a, c, e, and g are from one experiment and panels b, d, f, h are from an independent experiment. The bar in panel h indicates 50 μm for all panels in D and E.

FIG. 12 shows inhibitors of ARF function interfere with nuclear envelope breakdown. FIG. 12A shows that buffer (ELB) or ARF peptides (22 μM, final concentration) were added 15 minutes prior to the beginning of the assembly/disassembly assay. Samples were examined after 90 minutes of assembly conditions (interphase) and 75 minutes after cyclin was added (mitosis). In the merged images, DNA (Hoechst 33258) is shown in blue, NLS import substrate is shown in red, and membranes (DHCC) are shown in green. Numbers to the right of the panel indicate the percentage of intact nuclei at the 75 minute post-cyclin time-point; the bar in panel f indicates 50 μm for all panels in A and B. FIG. 12B shows that buffer (ELB) or Brefeldin A (BFA) were added 15 minutes prior to the beginning of the assembly/disassembly assay and analyzed as described in FIG. 12A.

FIG. 13 shows the prevention of nuclear envelope breakdown corresponds to a block in nuclear lamina disassembly. FIG. 13A shows that recombinant protein fragments (4 μg) were added to egg extract 15 minutes prior to the assembly/disassembly assay. The interphase samples were taken after 90 minutes of assembly and the mitotic samples were taken 75 minutes after the addition of cyclin. Detection of the lamins is shown in panels a-c and nuclear pore proteins reactive with mAb414 are shown in panels d-f. The samples were imaged with confocal microscopy. FIG. 13B shows ARF peptides (11 μM, final concentration) were incubated 15 minutes prior to the assembly/disassembly assay. Samples were analyzed as described in FIG. 13A. The bar in panel f indicates 50 μm for all panels in A and B.

FIG. 14 shows alignment of zinc fingers of human Nup153, Nup358, and Np14.

FIG. 15 shows results of the phage display screen of Example 2. Peptides chosen for further testing are boxed. The two shades of text indicate sequences derived from two different wash conditions in the third round of selection. Basic residues (H, R, and K) are also highlighted.

### III. DETAILED DESCRIPTION

The process by which a cell reproduces to create two identical copies is mitosis. The goal of mitosis is the formation of two identical cells from a single parent cell. The cells formed are known as daughter cells. In order for this to happen, the DNA in the chromosomes must be faithfully copied. This occurs via replication. The organelles, such as mitochondria, must also be distributed so that each daughter cell receives an adequate amount to function. The cytoplasm of the cell must also be physically separated into two different cells.

Cancer arises from a loss of normal growth control. In normal tissues, the rates of new cell growth and old cell death are kept in balance. In cancer, this balance is disrupted. This disruption can result from uncontrolled cell growth or loss of a cell's ability to undergo apoptosis. The process of cell division is driven by a cascading path of signals, with each event catalyzed by a unique enzyme. One strategy to impede the destructive course of the disease is the design and use of drugs that inactivate one of these enzymes, halting the division process.

Many of the features of cancer cells are due to defects in the genes that control cell division. The cell division process occurs as an orderly progression through four different stages. These four stages are collectively known as the cell cycle. Mitosis is the first stage. Mitosis occurs when a single cell divides into two daughter cells. In normal cells, the division produces two cells with the identical (or nearly identical barring replication mutations) genetic content of the parent cell. Mitosis is further broken down into sub-phases based on visible changes within the cells, especially within the nucleus.

In prophase, the nuclear envelope dissolves and the chromosomes condense in preparation for cell division. The chromosomes become more compact, allowing them to be more easily sorted into the forming daughter cells. Also in prophase, protein fibers (spindle fibers) form and reach from one end of the cell to another. This bundle of fibers gives the dividing cell the structure it needs to push and pull the cell components and form two new cells.

The protein strands that reach from one end of the cell to the other are called microtubules. These proteins are assembled and disassembled during the cell division process. They are the target of several different chemotherapy agents. For example, Taxol®, a chemical derived from an extract of the yew tree, binds to the microtubules and does not allow them to disassemble. This causes the cells to fail in the mitosis process and die. Another class of chemotherapy agent, represented by vinblastine, has the opposite effect. These drugs don't allow the spindle fiber to form. The result is the same, as the cell division process is inhibited.

Needed in the art are compositions and methods to inhibit cancer cell proliferation by inhibiting nuclear envelope breakdown.

A. Compositions and Methods

Nuclear envelope breakdown is a critical step in the cell cycle of higher eukaryotes. Integral membrane proteins associated with the nuclear membrane have been observed to disperse into the endoplasmic reticulum at mitosis. Disclosed herein is a role for the COPI coatomer complex in nuclear envelope breakdown, implicating vesiculation as an important step. It is shown herein that a nuclear pore protein, Nup153, plays a critical role in directing COPI to the nuclear membrane at mitosis and that this event provides feedback to other aspects of nuclear disassembly.

1. Nuclear Envelope

For much of the cell cycle, eukaryotic genomic DNA is enclosed by two membrane bilayers termed the nuclear envelope. Thousands of macromolecular nuclear pore complexes are present in this nuclear envelope and serve as conduits for traffic between the nucleus and cytoplasm (Vasu, S. K., and Forbes, D. J., Curr Opin Cell Biol 13, 363-375 (2001); Suntharalingam, M. and Wente, S. R., Dev. Cell 4:775-89 (2003)). Despite the continuity at each nuclear pore between the inner and outer nuclear membranes, a unique profile of proteins are anchored in the inner nuclear membrane through interactions with proteins located at the peripheral nuclear lamina and chromatin (Burke, B., and Ellenberg, J., Nat Rev Mol Cell Biol 3, 487-497 (2002)). In most metazoan cells, the nuclear envelope becomes reorganized at mitosis and ultimately its disassembly is key to accurate inheritance of both genomic DNA and nuclear envelope components.

Events proposed to be important to the disintegration of the nuclear envelope include: changes in nuclear pore configuration (Lenart, P., et al., J Cell Biol 160, 1055-1068 (2003); Terasaki, M., et al., Mol Biol Cell 12, 503-510 (2001)); creation of a tear in the nuclear envelope due to microtubule-dependent tension exerted on the nuclear envelope (Beaudoui, J., et al., Cell 108, 83-96 (2002); Salina, D., et al., Cell 108, 97-107 (2002)); and an increase in lateral mobility of integral membrane proteins allowing equilibration between the nuclear envelope and the endoplasmic reticulum and resulting in the loss of distinction between these membrane populations (Ellenberg, J., et al., J Cell Biol 138, 1193-1206 (1997); Yang, L., et al., J Cell Biol 137, 1199-1210 (1997)). Observations that there are specific sub-types of vesicle populations necessary for nuclear envelope formation in vitro (Buendia, B., and Courvalin, J. C., Exp Cell Res 230, 133-144 (1997); Drummond, S., et al., J Cell Biol 144, 225-240 (1999); Ewald, A, et al., Eur J Cell Biol 73, 259-269 (1997); Sasagawa, S., et al., Eur J Cell Biol 78, 593-600 (1999); Vigers, G. P., and Lobka, M. J., J Cell Biol 112, 545-556 (1991)) support the idea that formation of specialized vesicles occurs at mitosis. An alternative interpretation of this data is that specialized microdomains within the mitotic endoplasmic reticulum are the source of specialized vesicles, which are generated only when this membrane network is fragmented during biochemical fractionation (Collas, L, and Courvalin, J. C., Trends Cell Biol 10, 5-8 (2000); Ellenberg, J., et al., J Cell Biol 138, 1193-1206 (1997)). Indeed, recent models have discounted vesiculation as a step of nuclear envelope breakdown (Aitchison, J. D., and Rout, M. P. Cell 108, 301-304 (2002); Burke, B., and Ellenberg, J., Nat Rev Mol Cell Biol 3, 487497 (2002); Collas, I., and Courvalin, J. C., Trends Cell Biol 10, 5-8 (2000); Gonczy, P. Nuclear envelope: torn apart at mitosis. Curr Biol 12, R242-244 (2002); Lenart, P., and Ellenberg, J., Curr Opin Cell Biol 15, 88-95 (2003)).

Nuclear pore proteins (nucleoporins or Nups), situated in intimate connection with the nuclear membrane are uniquely positioned to help execute nuclear envelope breakdown. Nup153 is dynamically associated with the pore (Daigle, N., et al., J Cell Biol 154, 71-84 (2001), with a steady-state localization on the nuclear face of the pore and regions exposed on the cytoplasmic side as well (Fahrenkrog, B., et al., J Struct Biol 140, 254-267 (2002); Nakielny, S., et al., Embo J 18, 1982-1995 (1999); Pante, N., et al., J Cell Biol 126, 603-617 (1994); Sukegawa, J., and Blobel, G., Cell 72, 29-38 (1993); Walther, T. C., et al., Embo J 20, 5703-5714 (2001)). The function of Nup153 in nuclear envelope disassembly was explored, and a critical role for this pore protein in nuclear envelope breakdown was discovered. To better understand this novel role, proteins were identified that interact with the central region of Nup153 and an association between Nup153 and the COPI complex was found. This coatomer complex has been previously characterized in the context of vesicle budding both within the Golgi and between Golgi and ER (Nickel, W., et al., J Cell Sci 115, 3235-3240 (2002)). Perturbing the function of the COPI complex dramatically impairs nuclear envelope disassembly. These results lend insight into key players at this important stage of the cell cycle. Moreover, the machinery of vesicular trafficking is now definitively linked to nuclear envelope breakdown.

B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular protein, such as Nup153 or Nup358/RanBP2 is disclosed and discussed, and a number of modifications that can be made to a number of molecules including the amino acids of Nup153 or Nup358/RanBP2 are discussed, specifically contemplated is each and every combination and permutation of these amino acids and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-B, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

A composition comprising a molecule that inhibits nuclear envelope breakdown is disclosed. A molecule that inhibits nuclear envelope breakdown can be called an inhibitor of nuclear envelope breakdown, and nuclear envelope breakdown inhibitors reduce the breakdown, or disassembly, of the nuclear envelope. For example, they can reduce breakdown by greater than 1%, 10%, 20%, 30%, 40%, 50%, 60%, 70%, 80%, 90%, 100%, as compared to a control when tested in an in vitro nuclear disassembly assay as described herein. In certain embodiments the inhibitors of nuclear envelope breakdown can cause a 2-fold, 5-fold, 10-fold, 100-fold, 1000-fold, or 10,000-fold or greater breakdown as compared to a control, as discussed herein.

Inhibition can be measured, for example, by “percent protection.” By percent protection is meant the amount of nuclei that remain intact after mitotic signaling is initiated compared to the amount of nuclei in control samples that are also shifted to mitosis but in the presence of an inert protein control. This test is done at the earliest point at which control nuclei have disassembled. “Inhibition” is defined as perturbation of a normal cell cycle progression and/or kinetics and typically this is determined by comparison to a control.

In one embodiment, the molecule is not nocodozole, p50/dynamitin, or p62. In another embodiment, the molecule does not arrest the cell cycle prior to nuclear envelope breakdown. By “arrest cell cycle” is meant causing the cell cycle to stop progression and not complete the cycles of division. The arrest typically occurs at a specific stage of the cell cycle. In particular, the cell cycle can be arrested at interphase and not progress to mitosis.

The composition can interfere with a Nup153-COPI interaction. Interference means reducing a Nup153-COPI interaction. This interference in certain embodiments can occur directly where, for example, the composition acts as a competitive inhibitor of the Nup153 and COPI interaction. It can also occur indirectly, through for example, interaction with Nup153 or COPI allosterically, such that the COPI and Nup153 interaction cannot take place. “COPI complex” is defined as any complex containing at least one of the COPI subunits α, β, β′, δ, ε, γ, γ′, ζ1, or ζ2. A “COPI interaction” is defined as any interaction between at least one of the COPI subunits α, β, β′, δ, ε, γ, γ′ ζ1, or ζ2. Other examples of indirect interaction can occur by changing the conformation of Nup153 or COPI, stearically hindering their interaction while not directly interacting with the Nup153-COPI binding site, or by interfering with other proteins associated with the complex by inhibiting their interaction. Interfering with the Nup153-COPI interaction can also mean inhibiting nuclear envelope breakdown (disassembly). The inhibitors of Nup153-COPI interaction in certain embodiments can be compositions that reduce the interaction by for example, 10%, 20%, 30%, 40%, 50%, 60%, 70%, 80%, 90%, or 100%, as compared to a control Nup153-COPI interaction. In certain embodiments, a control interaction can be determined by incubating Nup153 and COPI. An example of the conditions that can be used for this interaction are found in Example 1. In this example, pulldown buffer was used (50 mM Hepes pH 7.8, 5 mM MgCl₂, 200 mM NaCl, 0.5% Triton X-100, 2 μg/ml aprotinin and leupeptin). In other embodiments, the inhibitors of interaction can reduce the interaction by, for example, 2-fold, 5-fold, 10-fold, 100-fold, or 1000-fold.

As disclosed herein, the zinc finger region interacts with a subunit of the COPI complex or protein linking the COPI complex and the Nup153. Furthermore, the C-terminal and N-terminal region surrounding the zinc finger region can act as binding sites for inhibitors, because by binding the N-terminal or C-terminal regions, for example, sterically inhibiting binding at the zinc finger region. The composition can bind the N-terminal, C-terminal, or zinc finger region of Nup153. Binding means that the interaction can be measured in an assay, such as an equilibrium dialysis assay, or gel shift, or chromatography, such that a binding or dissociation constant for the interaction can be obtained. For example, in certain embodiments bind can mean an interaction defined by a dissociation constant (k_{d}), for example, of less than or equal to 10⁻¹²M, 10⁻¹¹M, 10⁻¹⁰M, 10⁻⁸M, 10⁻⁹M, 10⁻⁷M, 10⁻⁶M, 10⁻⁵M, 10⁻⁴M, 10 ⁻³, or 10⁻². The primary structure of Nup153 can be divided into three regions; a unique N-terminal region, a central domain consisting of four to five zinc fingers (depending upon species) and a C-terminal region containing approximately 30 irregularly spaced FXFG repeats. Different classes of FG-containing repeats are found in many nucleoporins and often represent binding sites for nuclear import and export receptors, and the C-terminal region of Nup153 has indeed been shown to interact with several such receptors (Walther et al., EMBO J. 2001 October; 20 (20): 5703-5714). Variations of these FG regions are well understood in the art and are considered herein disclosed.

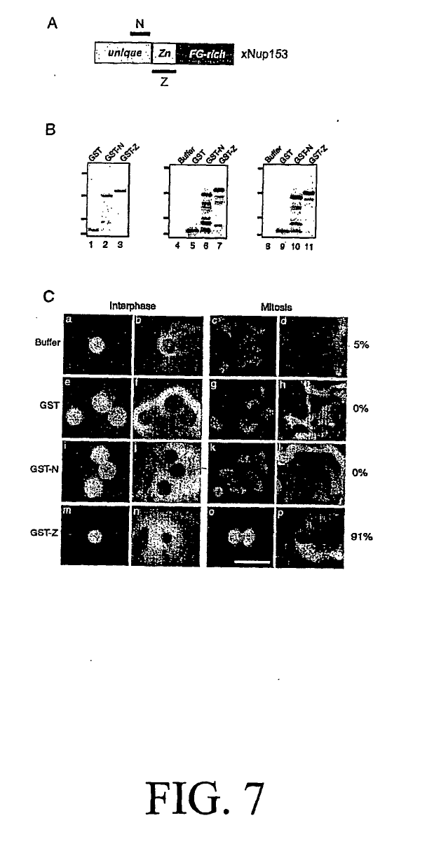

When a fragment encompassing the central zinc finger domain of Nup153 (FIG. 7, Example 1) was included in the cell-free extracts derived from Xenopus eggs, which were used to form synthetic nuclei around sperm chromatin, inhibition of nuclear envelope breakdown was apparent (91% of nuclei remaining). Membrane-staining rims around the DNA, typical of the nuclear envelope, were observed in the presence of the zinc finger. In addition, nuclear import cargo was still concentrated in nuclei under mitotic conditions, confirming both the presence and integrity of the nuclear envelope.

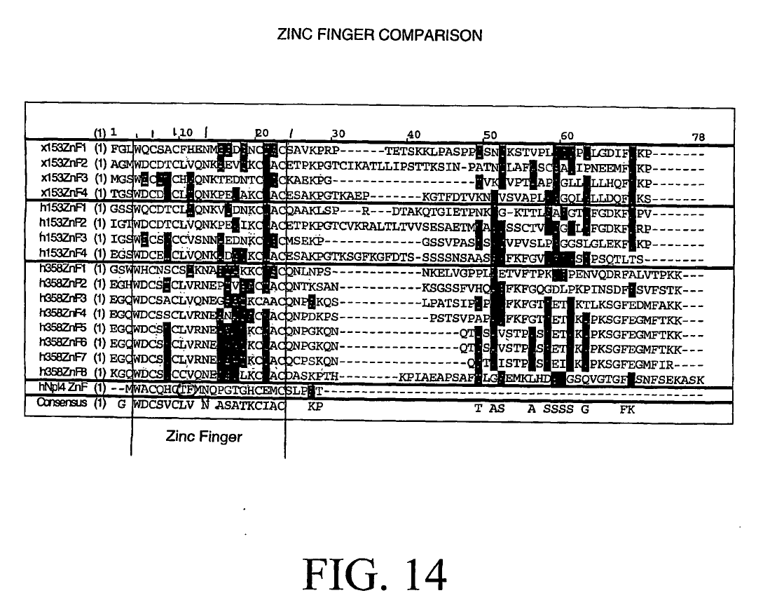

The composition can also bind the zinc finger region of Nup358/RanBP2 or Np14. In FIG. 14, sequence alignment data shows similarity between the zinc finger regions of Nup153, Nup358, and Np14. Nup358/RanBP2 is another vertebrate pore protein, showing that Nup358 probably also plays a role in recruitment of the COPI machinery. Np14, a protein that has been implicated in the process of nuclear assembly, also contains a zinc finger with similarity. (Hetzer, M., et al., Nat Cell Biol 3, 1086-1091 (2001)). Furthermore, the small GTPase Ran can interact with the zinc finger of Nup153 (Nakielny, S., et al., EMBO 18:1982-1995 (1999)); showing that Ran, which is known to play an important role in nuclear envelope assembly, is a candidate for modulating recruitment of the COPI complex to Nup153 (Example 1). SEQ ID NOS 8 and 10 represent Nup358 and Np14, respectively. The zinc finger region of Nup358 can be found at amino acids 1353-1811 of SEQ ID NO: 8, and at amino acids 583-608 of Np14. It is understood that in certain embodiments, GTPase Ran is not included as an inhibitor.

The composition can also bind any portion of a peptide, wherein the peptide comprises a sequence having at least 30%, 40%, 45%, 46%, 47%, 48%, 49%, 50%, 60%, 70%, 80%, 90%, or 100% identity to amino acids 1353 to 1811 of SEQ ID NO: 8 or amino acids 583 to 608 of SEQ ID NO: 10 (the zinc finger domains of Nup358 and Np14, respectively).

The composition can also bind a peptide, wherein the peptide comprises a sequence having at least 30%, 40%, 45%, 46%, 47%, 48%, 49%, 50%, 60%, 70%, 80%, 90%, or 100% identity to amino acids 658 to 891 of SEQ ID NO: 2 (the zinc finger domain of Nup153).

The composition can also bind a peptide, wherein the peptide comprises a sequence set forth in SEQ ID NOS: 12, 14, 16, 18, 20, 22, 24, and 26 (COPI coatomer proteins.)

It is understood that the when a composition is a peptide or peptide related molecule, such as an inhibitor of the Nup153-COPI complex, the peptide can be any length or less than any length, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 86, 87, 88, 89, 90, 91, 92, 94, 95, 96, 97, 98, 99, 100, 110, 120, 130, 140, 150, 160, 170, 180, 190, 200, 210, 220, 230, 240, 250, 300, 350, 400, or 500 amino acids or units in length. A Nup153-COPI complex is a complex of molecules that includes Nup153 and at least one of the subunits of COPI such as α, β, β′, δ, ε, γ, γ′ ζ1, or ζ2.

It is understood that in certain embodiments the inhibitors of nuclear envelope breakdown, such as inhibitors of Nup153-COPI interaction have relationship to the zinc finger region of Nup153. This region contains 5 zinc fingers connected by a variety of different linking regions. Thus, in certain embodiments two or more zinc fingers can be linked together to form an inhibitor. Molecules have this type can be represented by the formula I
-((Zₐ-Lₐ-Z_{b})-L_{b})ₙ-

Wherein -Zₐ represents a zinc finger as described herein.

Wherein -Lₐ represents a linker which can be anything.
For example, Lₐ can be made of amino acids, or derivatives or alkyl changes or nucleic acids. Linker materials are well known and can be used herein. Furthermore, the linker can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acid or units long.

Wherein -Z_{b} can be any zinc finger region as described herein.

Wherein -L_{b} represents a linker which can be anything. For example, L can be made of amino acids, or derivatives or alkyl changes or nucleic acids. Linker materials are well known and can be used herein. Furthermore, the linker can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 90, 100 or more amino acid or units long.

It is understood that there can be as many Zinc fingers as desired. For example, there can be 1, 2, 3, 4, 5, 6, 7, or 8 zinc fingers regions, and that these then would be connected by the appropriate number of linkers.

It is also understood that the inhibitors can be made of combinations of different types of molecules, such as an antibody, an aptamer, or some other type of molecule, that, for example, binds COPI. It is understood that these molecules can be linked in any combination, and that in Formula I in these embodiments, Zₐ or Z_{b} can be then also any other type of molecule, and can be represented as Xₐ or X_{b}. These molecule can be any molecule that binds COPI or binds Nup153 as described herein, for example.

It is also understood that the inhibitors as described herein can also be used in methods of modulating Nup153 or COPI activity, nuclear envelope breakdown, cell cycle progression, and cell viability. They can also be used as molecules to further refine better inhibitors or to isolate other molecules that interact with COPI or Nup153 in the cell.

Also disclosed are compositions that interact with the zinc finger region of Nup153, such as compositions identified using any selection mechanism, such as phage display or a two-hybrid system. For example, ZNFPHDI=CTTHPFTHECGGGS (SEQ ID NO: 30) was identified in a phage display experiment to a Nup153 zinc finger. This peptide in synthetic form as well as displayed form was capable of inhibiting nuclear envelope breakdown.

1. Homology/Identity

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

It is understood that one way to define any known variants and derivatives or those that might arise of the disclosed genes and proteins herein is through defining the variants and derivatives in terms of homology to specific known sequences. For example SEQ ID NO: 1 sets forth a particular sequence of a Nup153 nucleic acid, and SEQ ID NO: 2 sets forth a particular sequence of the protein encoded by SEQ ID NO: 1, a Nup153 protein. Specifically disclosed are variants of these and other genes and proteins herein disclosed which have at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level. For example, FIG. 14 shows sequence alignment data for Nup153, Nup358, and Np14. It can be seen that there is homology between these zinc fingers.

This class of zinc fingers has been referred to as the Np14 zinc finger/RanBP2 zinc finger domain or NZF (Wang et al., (J. Biol. Chem. 278:20225-20234, 2003). Hallmark features of this general category of zinc finger include the four regularly spaced cysteine residues (CX₂CX₁₀CX₂C) and an asparagine residue at position 8 relative to the first cysteine. A database search based on these criteria yielded 243 peptide sequences in 166 different proteins. Although a tryptophan at position −2 is shared by Np14, Nup153, and Nup358, it is not shared by all zinc fingers that match the other criteria. It was demonstrated that zinc was bound by this region of Np14.

Although the above criteria can be used to describe this category of zinc finger regions, one functional distinction between members of this group lies in their ability to recognize ubiquitin. In turn, this property correlates with the presence of particular amino acids (TF at positions 5 and 6 relative to the first cysteine).

Nup153 and Nup358/RanBP2 zinc fingers do not recognize ubiquitin and likewise do not contain the TF motif Instead, “LV” predominates at this position. The Nup153 and Nup358 zinc finger sequences contain other patterns in the amino acid composition that may distinguish this subclass. Specifically as shown in FIG. 14, F at −9, G at −4, an acidic residue [D/E] at −1, a basic residue [K/R] at +14, and aliphatic residues [A, G, I, L, V] at positions +16, 17.

There are also distinctions between the zinc finger regions of Nup153 and Nup358. For example, position +9 is acidic in Nup358 [E] and basic in Nup153 [K]. Position 11 is acidic in Nup153 and is predominated by serine or threonine in Nup358.

Another distinctive feature of the zinc finger domain of both Nup153 and Nup358/RanBP2 is its repetitive nature. In terms of function, while one zinc finger can inhibit nuclear disassembly, its activity is less robust than when in the native, repetitive context. Having more than one zinc finger and/or having certain linker sequences appears to be important to the recruitment of the inhibition of nuclear disassembly involving at least one subunit of the COPI complex, but not necessary. Although the linker regions themselves do not share tight similarity to each other, they are generally SIT rich and share some motifs (such as “KPG” at +22 in Nup53 and the other conserved residues at −9, −4 and −1 mentioned above).

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444(1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 81, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Nucleic Acids

There are a variety of molecules disclosed herein that are nucleic acid based, including for example the nucleic acids that encode, for example, Nup153 or Nup358, as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, Nup153 or Nup358, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of Nup153 or Nup358, or the genomic DNA of Nup153 or Nup358 or they can interact with the polypeptides thereof. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Also disclosed are compositions that comprise a molecule that inhibits nuclear envelope breakdown, wherein the molecule is an aptamer. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide.

For example, when determining the specificity of Nup153 aptamers, the background protein could be Nup358. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Other agents useful in inhibiting nuclear envelope breakdown include RNA interference (RNAi) or small interfering RNA (SiRNA). Methods of RNAi and SiRNA are described in detail in Hannon et al. (2002), RNA Interference, Nature 418:244-250; Brummelkamp et al. (2002), A System for Stable Expression of Short Interfering RNAs in Mammalian Cells, Science 296:550-508; Paul et al. (2002), Effective expression of small interfering RNA in human cells, Nature Biotechnology 20: 505-508, which are each incorporated by reference in their entirety for methods of RNAi and SiRNA and for designing and testing various oligos useful therein. Such methods could be directed at inhibiting nuclear envelope breakdown by interfering with the RNA of nuclear envelope associated proteins, such as Nup153 or Nup358.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozyrnes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10^{-10,} or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246,5,874,566, and 5,962,426.

External guide sequences (EGSs) are molecules that bind a target nucleic acid molecule forming a complex, and this complex is recognized by RNAse P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631(1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as nucleic acids of chimeric proteins of Nup153 or Nup358, into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291(1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533(1984); Varga et al., J. Virology 65:6061-6070(1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100(1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis., as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials maybe in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 3S:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in viva. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of, viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or er vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

5. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovinis, retroviruses, hepatitis-B virus and most preferably cytomegalovirus, or from heterologous mammalian promoters, e.g. β-actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4:1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100-270), the cytomegaloviris early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes B-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transfomied mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thynidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410413 (1985)). The three examples employ bacterial genes under eikaryotic control to convey resistance to the appropriate drug G418 or neomycin (geneticin), xgpt (mycophenolic acid) or hygromycin, respectively. Others include the neomycin analog G418 and puramrycin.

6. Peptides

a) Protein Variants

As discussed herein there are numerous variants of the Nup153 protein and Nup358 protein that are known and herein contemplated. In addition, to the known functional Nup153 and Nup358 variants, there are derivatives of these proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | Ala A |
| Allosoleucine | AIle |
| Arginine | Arg R |
| Asparagines | Asn N |
| Aspartic acid | Asp D |
| Cysteine | Cys C |
| Glutamic acid | Glu E |
| Glutamine | Gln Q |
| Glycine | Gly G |
| Histidine | His H |
| Isolelucine | Ile I |
| Leucine | Leu L |
| Lysine | Lys K |
| Phenylalanine | Phe F |
| Proline | Pro P |
| Pyroglutamic acid | Pglu |
| Serine | Ser S |
| Threonine | Thr T |
| Tyrosine | Tyr Y |
| Tryptophan | Trp W |
| Valine | Val V |

TABLE 2
Amino Acid Substitutions
Original Residue Exemplary Conservative Substitutions

Ala; Ser
Arg; Lys; Gln
Asn; Gln; His
Asp; Glu
Cys; Ser
Gln; Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 2 sets forth a particular sequence of Nup153 and SEQ ID NO: 8 sets forth a particular sequence of a Nup358 protein. Specifically disclosed are variants of these and other proteins herein disclosed which have at least, 40% 04 50% or 60% or 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipmaii, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 2 is set forth in SEQ ID NO: 1. Another nucleic acid sequence that encodes the same protein sequence set forth in SEQ ID NO: 8 is set forth in SEQ ID NO: 7. It is also understood that while no amino acid sequence indicates what particular DNA sequence encodes that protein within an organism, where particular variants of a disclosed protein are disclosed herein, the known nucleic acid sequence that encodes that protein in the particular sequence from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept ProtRes 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

7. Antibodies

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarily determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain Nup153 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Contemplated herein is a composition comprising a molecule that inhibits nuclear envelope breakdown, wherein the molecule is an antibody. The antibody can bind any molecule associated with nuclear envelope breakdown, including Nup153, Nup358, COPI, or any molecule associated with these. The antibody can bind the N terminal region of Nup153, the C-terminal portion of Nup153, or the zinc fingerportion of Nup153.

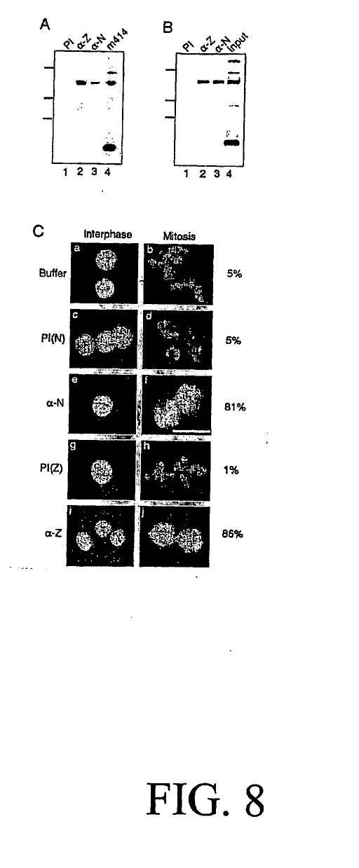

In Example 1, antibodies that specifically recognize Nup153 were used in a nuclear disassembly assay, as assessed by both immunoblot analysis and immunoprecipitation (FIG. 8). One antibody recognized the zinc finger region, and the other N-terminal region. Both antibodies were able to prevent the normal progression of events in disassembly. The nuclear membrane stayed largely intact (81-86% nuclei remaining at mitosis) as indicated by the accumulation of import cargo and membrane staining. Nuclear import mediated by a classical NLS took place as usual in the presence of these antibodies, indicating that there was no impediment to this particular pathway or to general movement through the nuclear pore complex. Although a difference in their potency existed, these two antibodies raised against different Nup153 domains interfered similarly with nuclear envelope breakdown, whereas antibodies purified from pre-immune sera had no effect on nuclear envelope breakdown.

The antibody in certain embodiments can also bind Nup358/RanBp2 or Np14. For example, an antibody that binds Nup358 can block nuclear envelope disassembly.

The antibody can comprise an antibody that binds a peptide, wherein the peptide comprises a sequence having at least 30%, 40%, 45%, 46%, 47%, 48%, 49%, 50%, 60%, 70%, 80%, 90%, or 100% identity to amino acids 658 to 891 of SEQ ID NO: 2 (the zinc finger domain of Nup153).

The antibody can also can also comprise an antibody that binds a peptide, wherein the peptide comprises a sequence having at least 30%, 40%, 45%, 46%, 47%, 48%, 49%, 50%, 60%, 70%, 80%, 90%, or 100% identity to amino acids 1353 to 1811 of SEQ ID NO: 8 or amino acids 583 to 608 of SEQ ID NO: 10 (the zinc finger domains of Nup358 and Np14, respectively).

The antibody can also bind a peptide, wherein the peptide interacts with a peptide comprising a sequence set forth in SEQ ID NOS: 12, 14, 16, 18, 20, 22, 24, 26, or 35 (COPI coatomer proteins.)

The antibodies can be either polyclonal or monoclonal, as described below.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al., and Boerner et al. are also available for the preparation of human monoclonal antibodies (Cole et al., Monoclonal Antibodies and Cancer Therapy, Alan R Liss, p. 77 (1985); Boerner et al., J. Immunol., 147(1):86-95 (1991)).

Disclosed are hybidoma cells that produces the monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises Nup153 or Nup358. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of Nup153 or Nup358 expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma 1998 December;17(6):569-76; Kilpatrick KE et al. High-affinity monoclonal antibodies to PED/PEA-15 generated using 5 μg of DNA. Hybridoma. 2000 August;19(4):297-302, which are incorporated herein by referenced in full for the the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of Nup153 or Nup358 antibody as fusion proteins. The antigen is produced by inserting a gene fragment in-frame between the signal sequence and the mature protein domain of the Nup153 or Nup358 antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against Nup153 or Nup358. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (EIISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for Nup153 or Nup358 and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific irnunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clak-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the intemction of the antibody with Nup153 or Nup358. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of the monoclonal antibody or fragment thereof and one or more reagents for detecting bindingofthe antibodyorfragment thereofto Nup153 or Nup358. The reagents can include, for example, fluorescent tags, enzymatic tags, or other tags. The reagents can also include secondary or tertiary antibodies or reagents for enzymatic reactions, wherein the enzymatic reactions produce a product that can be visualized.

8. Chimeric Proteins and Small Molecules

By “chimeric protein” is meant any single polypeptide unit that comprises two distinct polypeptide domains joined by a peptide bond, optionally by means of an amino acid linker, or a non-peptide bond, wherein the two domains are not naturally occurring within the same polypeptide unit. Typically, such chimeric proteins are made by expression of a cDNA construct but could be made by protein synthesis methods known in the art. A chimeric protein can comprise a fragment or derivative of a naturally occurring protein. The chimeric protein can also contain a mimetic of the naturally occurring protein. The distinct polypeptide domains can be in reverse orientation to those examples given herein, or in any order within the chimeric protein.

Contemplated is a composition comprising a molecule that inhibits nuclear envelope breakdown, wherein the molecule comprises a chimeric protein.

The composition can also be a small molecule. An example of a small molecule that can inhibit disassembly is brefeldin A. Brefeldin A is a fungal metabolite that inhibits the Guanine Exchange Factor for the small GTPase ARF1. This prevents ARF1 from switching into a form that binds tightly to membranes and, in turn, this interferes with the role that ARF1 plays in facilitating the recruitment of COP1 to membranes. Other small molecules can be identified or isolated as described herein using reagents the and methods described herein.

9. Antioxidants

Flavonoids, also known as “phenylchromones,” are naturally occurring, water-soluble compounds which have antioxidant characteristics. Flavonoids are widely distributed in vascular plants and are found in numerous vegetables, fruits and beverages such as tea and wine particularly red wine). Flavonoids are conjugated aromatic compounds. The most widely occurring flavonoids are flavones and flavonols (for example, myricetin, (3,5,7,3′,4′,5′,-hexahydroxyflavone), quercetin (3,5,7,3′,4′-pentahydroxyflavone), kaempferol (3,5,7,4′-tetrahydroxyflavone), and flavones apigenin (5,7,4′-trihydroxyflavone) and luteolin (5,7,3′,4-tetrahydroxyflavone) and glycosides thereof and quercetin).

10. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional fomms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticies, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immununol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214 6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parentally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antinicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fimaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing cancer cell proliferation or tumor growth, the efficacy of the therapeutic antibody can be assessed in various ways well known to the skilled practitioner. For instance, one of ordinary skill in the art will understand that a composition, such as an antibody, disclosed herein is efficacious in treating or inhibiting nuclear envelope dissassembly in a subject by observing that the composition reduces tumor growth or prevents a further increase in cancer cell proliferation

The compositions that inhibit nuclear envelope breakdown disclosed herein may be administered prophylactically to patients or subjects who have or are at risk for cancer. The disclosed compositions and formulations can be used to inhibit abberant cellular proliferation. For example, the disclosed compositions can be used to inhibit cell growth of cancer cells. This disclosed compositions can be used to inhibit cancer cell proliferation. Therefore, the cancer cells do not multiply. Thus, the compositions can be used to treat patients with cancer. It is understood that any therapeutic effect can be beneficial and that a patient does not need to cured to be treated. The compositions can be used to kill cancer cells. The killing of a cancer cell means that die cell not only does not divide, it also gets destroyed. It can be beneficial to both inhibit the growth of a cancer cell as well as kill a cancer cell. The disclosed compositions can be used to treat any disease where uncontrolled cellular proliferation occurs such as cancers.

The disclosed inhibitors can also be used in combination with any other composition that inhibits cellular proliferation, such as other anti-cancer compounds, such as vinblastin or doxorubcin or taxol.

Other molecules that interact with Nup153 or Nup358 to inhibit nuclear envelope breakdown which do not have a specific pharmacuetical function, but which may be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of cancer-related diseases.

11. Chips and Micro Arrays

Disclosed are chips where at least one address is the sequences or part of the sequences set forth in any of the nucleic acid sequences disclosed herein Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

12. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

13. Compositions Identified by Screening with Disclosed Compositions/Combinatorial Chemistry

a) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which the compositions disclosed in SEQ ID NOS: 1-26 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as Nup153 or Nup358, are also disclosed. Thus, the products produced using the combinatorial or screening approaches that involve the disclosed compositions, such as Nup153 or Nup358, are also considered herein disclosed.

It is understood that the disclosed methods for identifying molecules that inhibit nuclear envelope breakdown, for example, can be performed using high through put means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative mannerjust like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al., (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example, the zinc finger portion of Nup153, is attached to a DNA binding domain of a transcriptional activation protein, such as Gal 4. By performing the Two-hybrid technique on this type of system, molecules that bind the zinc finger portion of Nup153 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyiido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits U.S. Pat. Nos. 5,698,685 and 5,506,337), sulfamides (U.S. Pat. No. 5,618,825), and benzodiazepines (U.S. Pat. No. 5,288,514).

Screening molecules similar to Nup153 or Nup358 for inhibition of nuclear envelope breakdown is a method of isolating desired compounds.

As used herein combinatorial methods and libraries included traditional screening methods and libraries as well as methods and libraries used in iterative processes.

b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as Nup153 or Nup358, are also disclosed. Thus, the products produced using the molecular modeling approaches that involve the disclosed compositions, such as Nup153 or Nup358, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMn and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

14. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

15. Compositions with Similar Functions

It is understood that the compositions disclosed herein have certain functions, such as inhibiting nuclear envelope breakdown or binding Nup153 or Nup358. Disclosed herein are certain structural requirements for performing the disclosed functions, and it is understood that there are a variety of structures which can perform the same function which are related to the disclosed structures, and that these structures will ultimately achieve the same result, for example inhibition of Nup153.

C. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System IPlus DNA synthesizer (for example, Model 8700 automated synthesizer of Milligen-Biosearcb, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO: 2, is to link two or more peptides or polypeptides together by protein chemistry techniques. For example, peptides or polypeptides can be chemically synthesized using currently available laboratory equipment using either Fmoc (9-fluorenylniethyloxycarbonyl) or Boc (tert-butyloxycarbonoyl) chemistry. (Applied Biosysterns, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. For example, disclosed are nucleic acids in SEQ ID NOs: 1 and 3. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO: 1 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NOS: 1, 3, 5, 7, 9, 11, 13, 15, 17, 19, 21, 23, or 25, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO: 1 and a sequence controlling the expression of the nucleic acid.

Disclosed are nucleic acid molecules produced by the process comprising linking in an operative way a nucleic acid molecule comprising a sequence encoding a peptide set forth in SEQ H)NOS: 2, 4, 6, 8, or 10, for example, and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO: 2 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO: 2, wherein any change from the sequence are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the aniinal any of the nucleic acid molecules disclosed herein, wherein the animal is a mammal. Also disclosed are aniinals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

D. Methods of Identifying and Screening

Disclosed is a method of identifying a compound that inhibits nuclear envelope breakdown, comprising adding the compound to a system wherein the system comprises Nup153 and COPL wherein Nup153 and COPI can form a complex, and assaying for a molecule that decreases the amount of complex formed compared to the amount of complex formed in the absence of the compound. As disclosed above, by “inhibits nuclear envelope breakdown” is meant reducing the breakdown, or disassembly, of the nuclear envelope by greater than 1%, 10%, 20%, 30%, 40%, 50%, 60%, 70%, 80%, 90%, 100%, or 2-fold, 5-fold, 10-fold, 100-fold, 1000-fold, or greater. Nup153 and COPI can be human-derived, for example, or from a mammal, or from Xenopus laevis.

An egg extract system can be used as the method of identifying a compound. The egg extract system can be based on Xenopus laevis egg extract (Example 1).

As disclosed above, a high through put system, such as the FRET system described herein, can be used as the method of identifying a compound.

Inhibition can be monitored by percent protection, as described above.

Disclosed is a method of identifying a compound that modulates nuclear envelope breakdown, comprising adding the compound to a system wherein the system comprises Nup153, and assaying for a molecule that interacts with Nup153. By “interacts with Nup153” is meant effects, or affects, the function or structure of Nup153, either directly or indirectly, so that nuclear envelope breakdown is modulated. By “modulates” is meant has some affect upon, either directly or indirectly, to enhance, inhibit, or suppress nuclear envelope breakdown.

Also disclosed is a method of identifying and producing a compound, the method comprising bringing into contact a test compound and Nup153, assessing the activity of Nup153, comparing the activity of Nup153 when exposed to the test compound to activity of Nup153 in the absence of the test compound, wherein inhibition of Nup153 when exposed to the test compound identifies the test compound, and producing the identified test compound. The compound can be produced by the methods previously described.

For example, the disclosed compositions, such as Nup153, can be used as targets in a selection scheme disclosed herein to isolate the desired molecules. For example, a library of molecules could be incubated with Nup153, which is bound to a column. The molecules which are collected in the flowthrough after washing the column will be enriched for molecules that interact with Nup153 in a way that is inhibitory of nuclear envelope breakdown.

Inhibition can also be detected by gene array technology. Inhibition can also be detected by proteomic technology such as high throughput screening assays of protein interactions or activities.

Also disclosed is a method of identifying and producing an inhibitor of a COPI and Nup153 interaction, the method comprising bringing into contact a test compound, COPI, and Nup153, assessing the interaction of Nup153 and COPI, comparing the interaction of Nup153 and COPI when exposed to the test compound to the interaction of Nup153 and COPI in the absence of the test compound, wherein a reduction in the interaction of Nup153 and COPI when exposed to the test compound identifies the inhibitor, and producing the identified inhibitor. “Reduction in the interaction” is defined as a reduction of the interaction by less than 10%, 20%, 30%, 40%, 50%, 60%, 70%, 80%, 90%, or less than or equal to 100%.

Also contemplated is a method of identifying and producing a compound, the method comprising bringing into contact a test compound, a ligand of Nup153, and Nup153, assessing the interaction of Nup153 and its ligand, comparing the interaction of Nup153 and its ligand when exposed to the test compound to activity of Nup153 and its ligand in the absence of the test compound, wherein lack of interaction of Nup153 and its ligand when exposed to the test compound identifies the test compound, and producing the identified test compound.

Disclosed is a method of identifying a compound that modulates nuclear envelope breakdown, comprising adding the compound to a system wherein the system comprises Nup358/RanBP2, and assaying for a molecule that interacts with Nup358/RanBP2.

Also disclosed is a method of identifying and producing a compound, the method comprising bringing into contact a test compound and Nup358/RanBP2, assessing the activity of Nup358/RanBP2, comparing the activity of Nup358/RanBP2 when exposed to the test compound to activity of Nup153 in the absence of the test compound, wherein inhibition of Nup153 when exposed to the test compound identifies the test compound, and producing the identified test compound.

Also included is a method of identifying and producing a compound, the method comprising bringing into contact a test compound, a ligand of Nup358/RanBP2, and Nup358/RanBP2; assessing the interaction of Nup358/RanBP2 and its ligand; comparing the interaction of Nup358/RanBP2 and its ligand when exposed to the test compound to activity of Nup358/RanBP2 and its ligand in the absence of the test compound; wherein lack of interaction of Nup358/RanBP2 and its ligand when exposed to the test compound identifies the test compound; and producing the identified test compound.

By “bringing into contact” is meant the molecules are brought in physical proximity to one another, and can act upon each other either directly or indirectly.

Also contemplated is a method of identifying a test compound associated with nuclear envelope breakdown inhibition, comprising bringing into contact a test compound and a cell and identifying inhibition of nuclear envelope breakdown. This method can also include the step of using the test compound associated with nuclear envelope inhibition to identify mechanisms of nuclear envelope breakdown.

Disclosed is a method of identifying proteins that interact with Nup153 comprising operably linking Nup153 or a fragment of Nup153 to a DNA binding domain forming a first nucleic acid, transfecting a cell with the first nucleic acid, wherein the cell comprises a protein or protein fragment which is operably linked to an transcription activation domain, wherein the cell comprises a reporter system specific for the DNA binding domain, assaying the amount of expression from the reporter system, wherein an increase in expression indicates an interaction between the Nup153 or Nup153 fragment and the protein or protein fragment.

Examples of reporter systems include, but are not limited to, luciferase, GFP, RFP, or FTC, for example. Glow luminescence assays have been readily adopted into high throughput screening facilities because of their intrinsically high sensitivities and long-lived signals. The signals for chemiluminescence, bioluminescence, and calorimetric systems such as luciferase and beta-galactosidase reporter genes or for alkaline phosphatase conjugates are often stable for several hours.

Several commercial luminescence and fluorescence detectors are available that can simultaneously inject liquid into single or multiple wells such as the WALLAC VICTOR2 (single well), MICROBETA RTM JET (six wells), or AURORA VIPR (eight wells). Typically, these instruments require 12 to 96 minutes to read a 96-well plate in flash luminescence or fluorescence mode (1 min/well). An alternative method is to inject the test compound/Nup153 molecule into all sample wells at the same time and measure the luminescence in the whole plate by imaging with a CCD camera, similar to the way that calcium responses are read by calcium-sensitive fluorescent dyes in the FLIPR or FLIPR-384 instnuments. Other luminescence or fluorescence imaging systems include LEADSEEKER from AMERSHAM, the WALLAC VIEWLUX™ ultraHTS microplate imager, and the MOLECULAR DEVICES CLIPR imager.

PE BIOSYSTEMS TROPIX produces a CCD-based luminometer, the NORTHSTAR™ HTS Workstation. This instrument is able to rapidly dispense liquid into 96-well or 384-well microtiter plates by an external 8 or 16-head dispenser and then can quickly transfer the plate to a CCD camera that images the whole plate. The total time for dispensing liquid into a plate and transferring it into the reader is about 10 seconds.

Also disclosed are systems for assaying nuclear breakdown comprising Nup153, further comprising COPI, and further comprising a molecule from a Xenopus laevis egg extract Cell-free extracts derived from Xenopus eggs can be used to form synthetic nuclei around sperm chromatin (Example 1). This system offers the advantage of biochemical manipulation, while being well-established to robustly recapitulate both nuclear assembly as well as nuclear disassembly in response to mitotic signals (Murray, A. W. et al., Nature 339, 280-286 (1989)). Cycloheximide can be included to prevent synthesis of cyclin, thereby arresting these extracts in interphase, and cyclin added to induce mitotic signaling at a specific time in the assay.

Also disclosed is a system for assaying nuclear breakdown comprising Nup153, further comprising COPI, and further comprising a Xenopus laevis egg extract. The system can be a cell free system, or can take place in the cell. The system can comprise chromatin, such as sperm chromatin.

Also contemplated is a method of evaluating expression of Nup153, comprising contacting cells undergoing mitosis with a probe for Nup153, and detecting expression of Nup153. Expression of Nup153 can be detected using a fluorescent-based assay, or ELISA, for example.

E. Methods of Using the Compositions

1. Methods of Using the Compositions as Research Tools

The disclosed compositions can be used in a variety of ways as research tools. For example, the disclosed compositions, such as SEQ ID NOs: 2 and 12, 14, 16, 18, 20, 22, 24, and 26, can be used to study the interactions between COPI and Nup153, by for example acting as inhibitors of binding.

The compositions can be used for example as targets in combinatorial chemistry protocols or other screening protocols to isolate molecules that possess desired functional properties related to Nup153 or Nup358.

The disclosed compositions can also be used diagnostic tools related to cancer and other diseases related thereto.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining the role of Nup153 or Nup358 in cell division, particularly as it relates to COPI. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

Disclosed is a method of inhibiting nuclear envelope breakdown, comprising contacting a cell vvith a compound described herein, known to inhibit nuclear envelope breakdown. Nuclear envelope breakdown can be inhibited by inhibiting the activity of Nup153 or the interaction of Nup153 and COPI.

Also contenplated is a method of inhibiting a cell cycle of a cell comprising administering a Nup153 inhibitor to the cell. By “inhibiting a cell cycle” is meant disrupting the cell so that it does not proceed through the stages of cell division. Inhibition of cell cycle is defined as greater than 1%, 10%, 20%, 30%, 40%, 50%, 60%, 70%, 80%, 90%, or 100% inhibition of cell cycle.

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

The disclosed nucleic acids such as SEQ ID NO: 1, can be used to modify a gene of interest. The gene of interest is cloned into a vector designed for example, for homologous recombination. This gene could be, for example, a heterologous or synthetic regulatory sequence of Nup153.

2. Method of Treating Cancer

The disclosed compositions can be used to treat any disease where uncontrolled cellular proliferation occurs such as cancers.

Also disclosed is a method of treating a subject with cancer, comprising administering to the subject an effective amount of a compound that inhibits nuclear envelope breakdown. The compound can be any compound disclosed herein, which inhibits nuclear envelope breakdown.

A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fimgoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

F. Definitions

The present compounds, compositions, articles, devices, and/or methods are disclosed and described, are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “lo” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10“as well as “greater than or equal to “10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as betveen 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### The COPI Complex Functions in Nuclear Envelope Breakdown and is Recruited by the Nucleoporin Nup153

The Nucleoporin Nup153 Plays a Critical Role in Nuclear Envelope Breakdown

To investigate whetherNup153 participates in nuclear envelope breakdown, recombinant fragments of Nup153 were examined to determine if they exert dominant negative effects on this process in vitro. Cell-free extracts derived from Xenopus eggs were used to form synthetic nuclei around sperm chromatin. This system offers the advantage of biochemical manipulation, while being well-established to robustly recapitulate both nuclear assembly as well as nuclear disassembly in response to mitotic signals (Murray, A. W. et al., Nature 339, 280-286 (1989)). Cycloheximide was included to prevent synthesis of cyclin, thereby arresting these extracts in interphase. After nuclei were assembled, a recombinant form of stabilized cyclin B was added to initiate mitotic events. As expected, 20 to 40 minutes after addition of cyclin, the nuclei synchronously exhibited hallmark features of mitosis, including chromatin condensation and, after 75 minutes, the disappearance of a continuous nuclear membrane (FIG. 7C, panels c and d). Detection of import cargo provided the clearest visualization of whether a nuclear envelope was intact. When a protein fragment derived from the unique N-terminal domain of Nup153 (GST-N, FIGS. 7A and 7B, lanes 2 and 6) was present, nuclear envelope breakdown occurred without any marked alterations (FIG. 7C, panels k and 1). A control GST fusion protein (FIG. 7B, lanes 1 and 5) likewise had no effect (FIG. 7C, panels g and h). However, when a fragment encompassing the central zinc finger domain of Nup153 (GST-Z, FIGS. 7A and 7B, lanes 3 and 7) was included, a striking inhibition of nuclear envelope breakdown was apparent (91% of nuclei remaining). Membrane-staining rims around the DNA, typical of the nuclear envelope, were observed in the presence of zinc finger even after cyclin addition (FIG. 7C, panel p). In addition, nuclear import cargo was still concentrated in nuclei under mitotic conditions, confirming both the presence and integrity of the nuclear envelope (FIG. 7C, panel o). The effect of the Nup153 zinc finger was not due to selective stability of this fragment, as equivalent levels of recombinant protein were detected in each reaction by immunoblot following the last time point (FIG. 7B, lanes 9-11). It is noteworthy that nuclear assembly and NLS-mediated import proceeded in the presence of the zinc finger fragment, although there was a reduction in nuclear size (FIG. 7C, panels m-p).

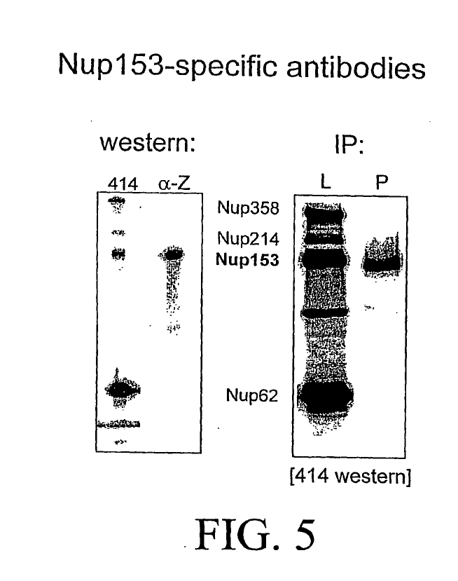

To be certain that the block imposed by this fragment was due to interference with a function attributable to Nup153, antibodies that specifically recognize Nup153 were used, as assessed by both immunoblot analysis (FIG. 8A, lanes 2 and 3) and immunoprecipitation (FIG. 8B, lanes 2 and 3). When these antibodies were included in the nuclear disassembly assay, they too prevented the normal progression of events: the nuclear membrane stayed largely intact (81-86%. p. 6 nuclei remaining at mitosis) as indicated by the accumulation of import cargo (FIG. 8C, panel f andj) and membrane staining. Of note, nuclear import mediated by a classical NLS took place as usual in the presence of these antibodies FIG. 8C, panels e and i), indicating that there was no impediment to this particular pathway or to general movement through the nuclear pore complex, consistent with previous studies (Ullman, K. S., et al., Mol Biol Cell 10,649-664 (1999)). Although a difference in their potency existed, these two antibodies raised against different Nup153 domains interfered similarly with nuclear envelope breakdown, whereas antibodies purified from pre-immune sera had no effect on nuclear envelope breakdown (FIG. 5C, panels d and h).

Nup153 Associates with Members of the COPI Coatomer Complex

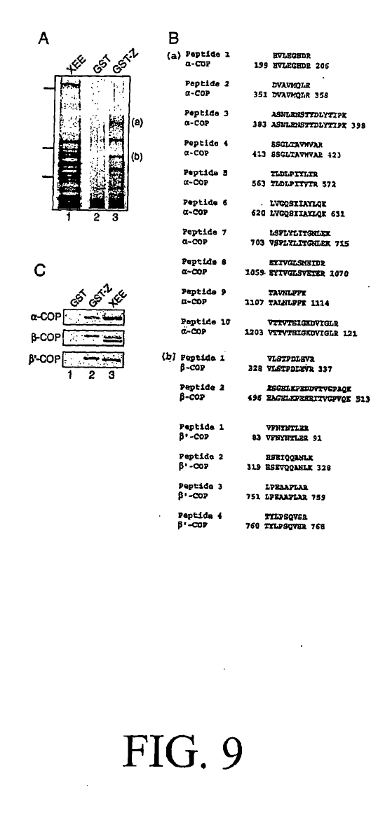

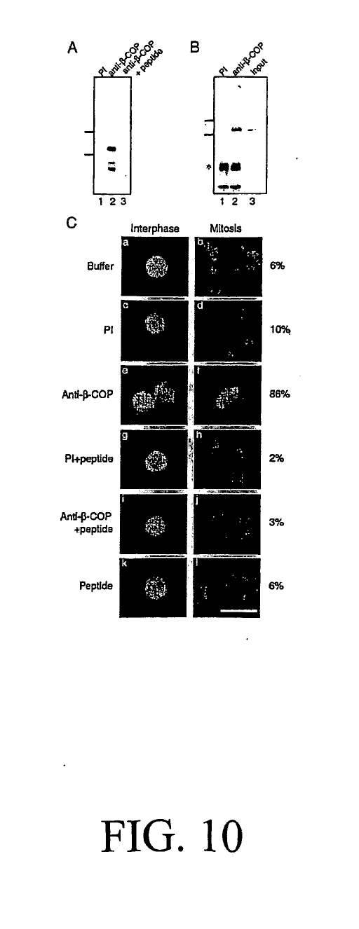

Although various proteins are known to associate with Nup153, none provide a clear link to nuclear disassembly (Hang and Dasso, J Biol Chem 277, 19961-19966, 2002; Moroianu, J., et al., Proc Natl Acad Sci USA 94, 9699-9704 (1997); Nakielny, S., et al., Embo J 18, 1982-1995 (1999); Shah, S., and Forbes, D. J., Curr Biol 8, 1376-1386 (1998); Shah, S., et al., J Cell Biol 141, 31-49 (1998); Smythe, C., et al., Embo J 19, 3918-3931 (2000); Vasu, S., et al., J Cell Biol 155, 339-354 (2001); Zhang, H., et al., Mol Cell Biol 22, 6498-6508 (2002)). To search for partners that would lend insight into how the zinc finger domain exerts its dominant negative effect, recombinant zinc finger protein was immobilized on sepharose beads and incubated in Xenopus egg extract. After several rounds of washes, proteins that remained associated were analyzed and compared to a parallel preparation using immobilized GST (FIG. 9A, lanes 2 and 3). Mass spectrometry analysis revealed that three members of the COPI coatomer complex (β, β′ and α) specifically associated with the Nup153 zinc finger region (FIG. 9B). Other members of this complex would not have been detected, as we had concentrated our efforts on proteins larger than 55 kDa due to the interference of the fusion protein in the analysis of smaller proteins. Identification of COPI components in association with the zinc finger domain of Nup153 was confirmed by immunoblot analysis of similarly prepared samples (FIG. 9C). Two bands were observed when the antibody against human β-COP was used to detect Xenopus proteins (FIG. 9C, lane 3) and only the upper band appears to associate with the zinc finger fragment. The analogous region of Xenopus β-COP detects one major product (see below, FIG. 10).

To determine the significance of the interaction between the coatomer proteins and this nucleoporin domain, the role of the COPI complex in nuclear disassembly was evaluated. Antibodies that immunoprecipitate human COPI components did not work well in immunoprecipitations from egg extract, indicating that their recognition of native Xenopus coatomer proteins was inefficient. An antibody was raised using a peptide derived from the sequence of Xenopus β-coatomer. These antibodies detect β-coatomer among a complex mixture of proteins on an immunoblot (FIG. 10A, lane 2) and recognize native protein in immunoprecipitations (FIG. 10B, lane 2). Addition of these antibodies to the nuclear disassembly assay prevented nuclear envelope breakdown (86% nuclei remaining at mitosis, FIG. 10C, panel f). This protective effect was blocked by inclusion of the immunogenic peptide (FIG. 10C, panel j) and was not observed with antibodies isolated from pre-immune sera (FIG. 10C, panel d). This leads us to conclude that β-coatomer, and likely the entire COPI complex, plays a critical role in disassembly of the nuclear envelope.

COPI Recruitment to Nuclear Membranes is Facilitated by Nup153 and Dependent on ARF

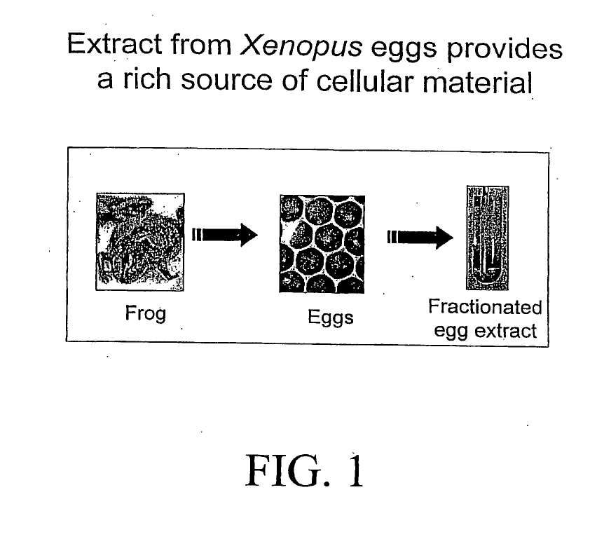

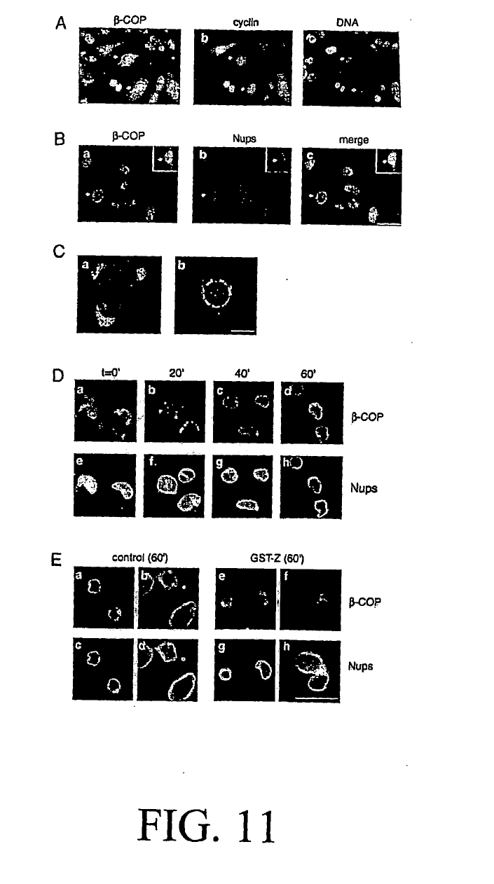

COPI appears to be recruited to the nuclear envelope at an early stage of mitosis. Indeed, the Golgi machinery is known to disperse around the nuclear periphery during prophase. However, the dynamic distribution of the Golgi has not been followed in relation to markers of the nuclear envelope or nuclear pores themselves. To investigate the localization of β-coatomer at different stages of the cell cycle, we first probed for its localization in conjunction with that of cyclin B in HeLa cells. Indirect immunofluorescence of cells in interphase reveals cyclin B in a diffuse staining pattern and β-coatomer predominantly in a pattern distinctive of the Golgi apparatus, clustered intensely at one side of the nucleus (FIG. 1A, panels a and b). In cells at early prophase, as determined by the appearance of cyclin B in the nucleus, dramatic reorganization of the Golgi apparatus is clear, with β-COP found in a rim around the nucleus (FIG. 11A, panels a and b, cells indicated by arrows). To determine whether this localization of coatomer coincides with the nuclear envelope, β-COP and FG-rich nucleoporins were simultaneously visualized. This analysis revealed a close juxtaposition of β-COP and nucleoporins (FIG. 11B, cells indicated by arrows). This observation is further underscored using confocal microscopy, where there is a clear distinction between the position of the Golgi relative to the nucleus at interphase (FIG. 11C, panel a) and the more intimate proximity seen as the Golgi disperses around the nucleus (FIG. 11C, panel b).

To further probe the connection between the population of COPI that colocalizes with the nuclear envelope and the process of nuclear envelope breakdown, nuclei assembled in the Xenopus egg extract was used. First, the kinetic relationship between the appearance of β-COP at the nuclear rim and nuclear envelope breakdown was examined. Before cyclin is added, some β-coatomer is seen by indirect immunofluorescence, likely due to its presence on endoplasmic reticulum or membranes associated superficially with the nuclei (FIG. 11D, panel a). Under these same conditions, an antibody directed against nuclear pore proteins (mAb414), clearly decorates the nuclear rim (panel e). Forty minutes after triggering mitosis by cyclin addition, a rim stain similar to that seen for nucleoporins was detected with antibodies directed against β-coatomer (FIG. 11D, panel c and g). This localization persisted at 60 minutes (panel d), immediately prior to nuclear envelope breakdown. The specificity of this detection was confirmed by blocking reactivity of the antibodies with the immunogenic peptide. To look at the role that Nup153 plays with respect to COPI, whether the zinc finger region of Nup153 alters the localization pattern of COPI at the nuclear envelop was examined. Nuclei were assembled in the presence of GST or GST-Z and the reactions were shifted to mitosis by the addition of cyclin. Sanples that contained the zinc finger domain, and hence held at a stage prior to nuclear envelope breakdown, showed reduced recruitment of β-coatomer to the nuclear envelope 60 minutes post cyclin addition (FIG. 11E, panels e, f compared to a, b). In these same samples, normal levels of mAb414-reactive nucleoporins were observed at the nuclear rim (FIG. 11E compare panels g, h to c, d). Thus, this region of Nup153 exerts a dominant negative effect on nuclear envelope breakdown by reducing recruitment of the COPI complex.

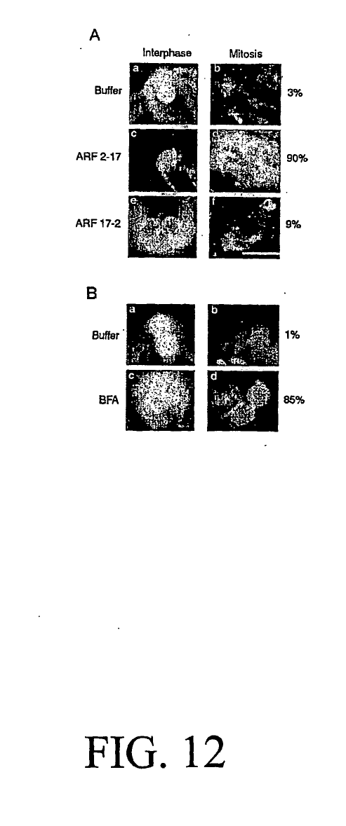

As a further test of the proposed role for COPI in nuclear envelope breakdown, a peptide was used which was derived from the small GTPase ARF1 (amino acids 2-17), that has previously been shown to interfere with ARF1-dependent coatomer recruitment (Kahn, R. A., et al., J Biol Chem 267, 13039-13046 (1992)). Nuclear assembly proceeded in the presence of this peptide: a continuous nuclear envelope with functional pores was observed (FIG. 12A, panel c). In contrast, inclusion of the ARF peptide clearly influenced nuclear envelope breakdown, with 90% of the nuclei remaining intact 75 minutes after cyclui addition (FIG. 12A, panel d). Nuclei in this particular reaction were found to have blebbing of the nuclear envelope, which has been found to correlate with robust nuclear formation activity (Powers, M. A., et al., J Cell Biol 128, 721-736 (1995)). A control peptide, composed of the reverse sequence, had no significant affect on nuclear disassembly (FIG. 12A, panel f). These results indicate that efficient nuclear envelope breakdown requires the participation of ARF. This conclusion is further corroborated by the finding that Brefeldin A, a small molecule that inhibits the exchange factor for ARF, interferes with nuclear envelope breakdown (FIG. 12B, panel d). Thus, the machinery involved in COPI recruitment to the nuclear membrane appears to consist of a prototypic member of the well-characterized coat forming cycle, ARF, as well as a new and unexpected modulator of this process, Nup153.

COPI-Mediated Recruitment to the Nuclear Envelope Influences Specific Cell Cycle Regulated Events

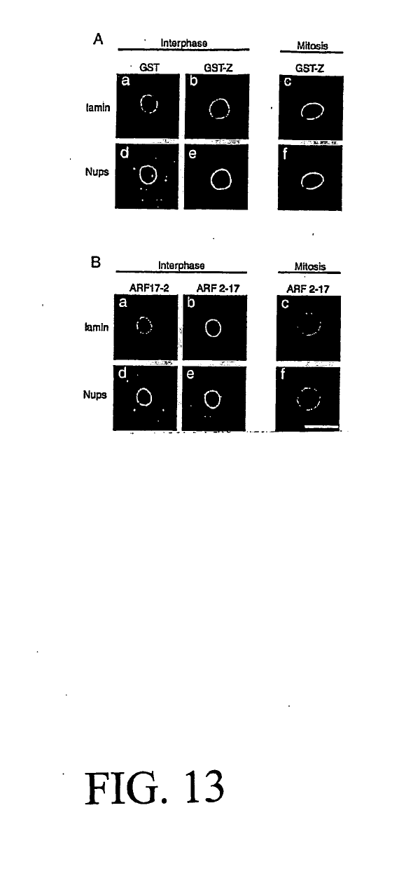

Having discovered several ways of interfering with nuclear envelope breakdown in the course of these studies, the interconnections between mitotic events were probed. For instance, it was noted that chromosome condensation appeared to take place in samples where nuclear envelope breakdown was inhibited (for example, see FIG. 10, panel f). To gain further insight into the question of how nuclear envelope breakdown is integrated with other events at this stage of the cell cycle, nuclear lamina disassembly was examined to determine if it proceeded under conditions in which nuclear envelope breakdown was inhibited. Indirect immunofluorescence revealed that the nuclear lamina appears intact in nuclei that persist under mitotic conditions in the presence of the Nup153 zinc finger fragment (FIG. 13A, panel c). Interestingly, this was also the case when disassembly was blocked by inclusion of ARF-inhibitory peptides (FIG. 13B, panel c). This suggests that a mechanism exists to couple recruitment of COPI to the nuclear envelope with disassembly of the nuclear lamina.

Results

Nucleoporins: New Players in Nuclear Envelope Breakdown

Global changes at the nuclear pore have been shown to precede nuclear envelope breakdown (Lenart, P., et al., J Cell Biol 160, 1055-1068 (2003); Terasaki, M., et al., Mol Biol Cell 12, 503-510 (2001)). Pore components have also been proposed to have a role early in prophase as anchor points at the nuclear surface for a dynein-dynactin complex (Aitchison, J. D., and Rout, M. P. Cell 108, 301-304 (2002); Beaudouin, J., et al., Cell 108, 83-96 (2002); Salina, D., et al., Cell 108, 97-107 (2002)). The functional connection reported here between Nup153 and COPI illustrates a new way in which a nucleoporin can contribute to nuclear disassembly. It remains possible that Nup153 plays other important roles at this step via additional mechanisms such as transport regulation. Indeed, by participating at more than one level in nuclear envelope breakdown, nucleoporins can help coordinate different mechanisms that underlie nuclear division.

Not only does Nup153 play arole in COPI recruitment, Nup153 predominantly localizes on the nuclear basket structure of the nuclear pore (Pante, N., et al., J Cell Biol 126, 603-617 (1994); Walther, T. C., et al., Embo J 20, 5703-5714 (2001)). The coatomer complex may gain access to Nup153 through traditional import mechanisms or via mitosis-specific alterations in nucleocytoplasmic flux. It is also noteworthy that Nup153, although considered a component of the nuclear pore basket, is exposed on the cytoplasmic side of the pore as well (Fahrenkrog, B., et al., J Struct Biol 140, 254-267 (2002); Nakielny, S., et al., Embo J 18, 1982-1995 (1999)). The zinc finger domain itself does not appear to be accessible on the cytoplasmic face of the pore (Fahrenkrog, B., et al., J Struct Biol 140, 254-267 (2002)), but reconfiguration of the pore early in mitosis (Lenart, P., et al., J Cell Biol 160, 1055-1068 (2003)) could lead to its exposure and the opportunity for direct access with the COPI complex. Finally, large-scale perforations in the nuclear envelope, shown to occur in somatic cells at mitosis (Beaudouin, J., et al., Cell 108, 83-96 (2002); Salina, D., et al., Cell 108, 97-107 (2002)), suggest that, at least after these initial events, there is ample opportunity for cytoplasmic components to access nuclear binding partners. These and other possibilities are not mutually exclusive and illustrate the range of mechanisms that could promote the partnership between Nup153 and coatomer proteins.

The specific domain of Nup153 that has been identified in this study to participate in nuclear envelope breakdown also gives rise to interesting implications. Such a zinc finger is found in one additional vertebrate pore protein, Nup358/RanBP2, suggesting that Nup358 also plays a role in recruitment of the COPI machinery. A more distantly related zinc finger motif is found in Np14, a protein that has been implicated in the process of nuclear assembly (Hetzer, M., et al., Nat Cell Biol 3, 1086-1091 (2001)). Interestingly, the zinc finger of Np14 was recently demonstrated to interact with ubiquitin (Meyer, H. H., et al., Embo J 21, 5645-5652 (2002); Wang, B., et al., J Biol. Chem. (2003)). While a zinc finger derived from Nup358 does not bind ubiquitin (Meyer, H. H., et al., Embo J 21, 5645-5652 (2002)), these studies raise the question of whether a ubiquitin-related modification is a determinant of interactions with the Nup153/Nup358 class of zinc finger. Also of note is the observation that the small GTPase Ran can interact with the zinc finger of Nup153 (Nakielny, S., et al., Embo J 18, 1982-1995 (1999)), suggesting that Ran, which is known to play an important role in nuclear envelope assembly, is a candidate for modulating recruitment of the COPI complex to Nup153.

Integrating Vesiculation into a Working Model of Nuclear Envelope Breakdown

Previous reports have pointed toward a thorough dispersal of integral nuclear membrane proteins into the ER (Ellenberg, J., et al., J Cell Biol 138, 1193-1206 (1997); Yang, L., et al., J Cell Biol 137, 1199-1210 (1997)). Since vesicles did not appear to be an end-point of nuclear envelope breakdown in these studies and because lateral movement between the nuclear envelope and ER membranes could explain the observed absorption of nuclear envelope into the ER, a role for vesiculation has been largely discounted (Burke, B., and Ellenberg, J., Nat Rev Mol Cell Biol 3, 487-497 (2002); Collas and Courvalin, Trends Cell Biol 10 5-8, 2000). The results here, however, show that vesicles formed from the nuclear envelope by a COPI-mediated pathway could in fact be an additional mechanism for dispersing the contents of the nuclear envelope into the ER via consequent fusion with the ER (see Yang et al., J Cell Biol 137, 1199-1210 1997). Indeed, electron microscopic data, in which vesicles were seen at the nuclear envelope, supports this conclusion (Cotter, L. A., et al., Scanning 20, 250-251 (1998).

Breakdown of the Golgi apparatus, like the nuclear envelope, appears to be the result of multiple mechanisms (Rossanese, O. W., and Glick, B. S., Traffic 2, 589-596 (2001); Warren, G., and Shorter, J., Annu Rev Cell Dev Biol. (2002)). One common theme in the current views of Golgi disassembly is that COPII-mediated ER to Golgi traffic is blocked at mitosis (Featherstone, C., et al., J Cell Biol 101, 2036-2046 (1985)) while COPI mediated budding from the Golgi continues or even escalates during the initial stages of mitosis (Zaal, K. J., et al., Cell 99, 589-601 (1999)). COPI plays an analogous role in facilitating nuclear envelope dispersal. Specifically, we have found that functionally interfering with the COPI machinery prevents nuclear disassembly (FIG. 10). It has been observed that during reorganization of the Golgi apparatus at mitosis, there is a stage at which the fragmented Golgi closely aligns with the nuclear envelope (FIG. 11A-C). Similar results were obtained using a different marker of the Golgi α-mannosidase) and a different cell line, BHK, indicating that this is a general phenomenon and that the Golgi stacks themselves, and not just components of the COPI complex, are closelyjuxtaposed with the nuclear envelope. This close proximity is consistent with an additional role for the COPI complex at the nuclear envelope at this stage of the cell cycle. β-COP recniitment to the nuclear envelope was also observed during early mitosis in an in vitro nuclear assembly/disassembly system (FIG. 5D) and, moreover, find that the zinc finger fragment of Nup153 interferes with this recruitment step (FIG. 5E). A role for the coatomer machinery in nuclear envelope breakdown does not preclude the notion that other mechanisms contribute to this process. Rather, this information indicates that multiple mechanisms provide a fail-safe mode of nuclear envelope breakdown, reflecting the importance of faithful segregation of the nuclear envelope and the genomic content itself.

Parallels Between the Nuclear Envelope and the Golgi

Consistent with commonalities observed between Golgi and nuclear envelope disassembly, these organelle membranes also share specific aspects of biogenesis machinery. Nuclear envelope closure requires the AAA-ATPase, p97, and its partners Np14/Ufdl (Hetzer, M., et al., Nat Cell Biol 3, 1086-1091 (2001)). Then, during nuclear envelope growth, p97 is again required but now in conjunction with a different partner, p47 (Hetzer, M., et al., Nat Cell Biol 3, 1086-1091 (2001)). p97, along with p47, has been previously implicated in formation of Golgi and transitional ER following mitosis (Acharya, U., et al., Cell 82, 895-904 (1995); Kondo, H., et al., Nature 388, 75-78 (1997); Rabouille, C., et al., Cell 82,905-914 (1995)). Tis close connection between Golgi-ER biogenesis/trafficking and nuclear envelope formation is further underscored by recent results in S. cerevisiae, which demonstrate altered nucleoporin distribution in mutants of several proteins involved in ER/Golgi trafficking (Ryan, K. J., and Wente, S. R., BMC Genet 3, 17 (2002). In contrast to these recently recognized corollaries, past studies focused on the role of ARF1 had led to the conclusion that this small GTPase does not have a critical role in nuclear envelope assembly or breakdown (Gant, T. M., and Wilson, K. L., Eur J Cell Biol 74, 10-19 (1997)). The ARF peptide used in these experiments (FIG. 12A) provided a more complete block to ARF function than did size-dependent ARF depletion (Gant, T. M., and Wilson, K. L., Eur J Cell Biol 74, 10-19 (1997)). A depletion approach can be complicated by the biological activity of residual protein even at extremely low concentration. This is well-illustrated by early studies of mitotic extracts in which an overall 10-fold dilution delayed complete nuclear breakdown only two-fold (Newport, J., and Spann, T., Cell 48,219-230 (1987)). Having obtained several lines of evidence for a role of ARF and coatomer, whether Brefeldin A affects nuclear envelope breakdown was examined. While very clear interference of nuclear envelope breakdown occurred (FIG. 12B), alternative mechanisms that contribute to this process may eventually lead to disassembly even when COPI recruitment has been compromised. Thus, an effect of Brefeldin A may be less evident as the extract progresses through mitosis and, similarly, could be masked to some extent in somatic cells, where it is clear that a combination of mechanisms leads to efficient nuclear envelope breakdown.

Cell Cycle Control of Nuclear Envelope Breakdown

This and other recent studies have highlighted that, far from being a passive process, nuclear envelope breakdown relies on a critical set of activities. Moreover, the observation that prevention of the COPI-mediated step in nuclear envelope breakdown leads to a parallel arrest in nuclear lamina breakdown (FIG. 13) points toward a mechanistic link between these particular events. In previous studies, lamina solubilization was demonstrated to take place under conditions where factors necessary for nuclear envelope breakdown had been titrated to prevent disassembly of the nuclear envelope (Newport, J., and Spann, T., Cell 48, 219-230 (1987)). A model consistent with these previous results, as well as with the results presented here, is that recruitment of COPI complexes to the nuclear pore provides a permissive signal for nuclear lamina breakdown. In the previous study, this initial recruitment step could have occurred even when factors necessary to complete nuclear envelope breakdown were limiting. In contrast, in the experiments reported here, the recruitment step itself was targeted for inhibition, consequently preventing the hypothetical pennissive signal for lamina disassembly.

Materials and Methods

Recombinant Protein Production and Purifcation

Recombinant protein was induced with 1 mM isopropylthio-β-D-galactoside (IPTG) for 3 hours at 37° C. Bacteria were lysed by two different methods, either in the presence of sarkosyl to promote solubilization (Frangioni, J. V., and Neel, B. G., Anal Biochem 210, 179-187 (1993)) or in a PBS-based buffer with lysozyme and deoxycholate. The GST fusion proteins were purified using glutathione-Sepharose 4B resin (Amersham, Piscataway, N.J.) according to the manufacturer's protocol. Constructs encoding amino acids 426-655 (GST-N) and 655-926 (GST-Z) were gifts. Amino acid numbers are based on conceptual translation of the Xenopus N-terminus [Genbank accession AF434196 Dimaano, C., et al., J Biol Chem 276, 45349-45357 (2001)] combined with the original clone [Genbank accession AF045567 (Shah, S., et al., J Cell Biol 141, 3149 (1998))].

Generation and Affinity Purifcation of Antibodies

The purified recombinant Nup153-N and Nup153-Z fragments were used to produce immune antisera at Zymed Laboratories (South San Francisco, Calif.). The antibodies were then affinity purified following standard procedures using a matrix with covalently coupled fragment. Pre-immune antiserum was protein A purified following standard procedures. Antiserum against a synthetic internal peptide of Xenopus β-COP, (C)ESGELKPEDDVTVGPAQK (SEQ ID NO: 27) was also generated in rabbits at Zymed Laboratories. This peptide sequence was obtained in our mass spectrometry analysis and corresponds to amino acid residues 496-513 of human β-COP, accession #NP_{—}057535). A cysteine residue was included at the N-terminus of the peptide to allow coupling to the carrier for immunization as well as to SulfoLink sepharose (Pierce, Rockford, Ill.) for affnity purification.

Preparation of Xenopus Egg Extracts

Interphase Xenopus egg extracts were prepared using egg lysis buffer (ELB: 250 mM sucrose, 50 mM KCl, 2.5 mM MgCl₂, 10 mM Hepes pH 7.4) supplemented with 1 mM dithiothreitol (DTT), 5 μg/ml cytochalasin B, aprotinin and leupeptin (10 μg/ml each), and 50 μg/ml cycloheximide (Powers, et al., In Current Protocols in Cell Biology (New York, John Wiley & Sons), pp. 11.10.11-11.11.24, (2001). Eggswere lysed by centrifugation for 15 min at 10,000 rpm. The cytoplasmic layer was collected as crude extract. For preparation of fiactionated egg extract, the crude extract was further centrifuged for 1.5 hr at 268,000 x g. The clarified supernatant was removed and further centrifuged for 1 hr at 259,000 x g to remove residual membranes. The crude extract was stored in liquid nitrogen; fractionated extract was frozen in liquid nitrogen and then stored at −80° C.

GST Pulldown and Identification of Associated Proteins

50 μg of GST or GST-Z was loaded onto 15 μl bead volume of pre-equilibrated glutathione-Sepharose for 1 hr in 300 μL of pulldown buffer (50 mM Hepes pH 7.8,5 mM MgCl₂, 200 mM NaCl, 0.5% Triton X-100, 2 μg/ml aprotinin and leupeptin) which was used through-out this procedure. After washing with 3×1 ml, the beads were incubated with 50 μl of fractionated egg extract in a total volume of 500 μl, and the mixture was rotated for 2 hr at room temperature. The beads were then washed with 4×1 ml buffer and 1×1 ml of PBS. The bound proteins were eluted with 100 mM glycine, pH 2.5, precipitated with trichloroacetic acid and separated on 6.3% SDS-PAGE gel. Proteins were stained using the SilverQuest silver staining kit (Invitrogen, Carlsbad, Calif.). Gel bands containing binding proteins were excised, washed with 50% acetonitrile, and stored at −80° C. before sending out for peptide sequencing. Sequence analysis was performed at the Harvard Microchemistry Facility by microcapillary reverse-phase. HPLC nano-electrospray tandem mass spectrometry (μLC/MS/MS) on a Finnigan LCQ DECA quadrupole ion trap mass spectrometer.

Immunoprecipitation and Immunobloting

Immunoprecipitation and imnimunoblotting were performed following standard procedures. Antibodies used were obtained as follows: anti-GST was a gift, mAb414 which recognized Nup62, Nup153, Nup214, and Nup358 (Covance, Richmond, Calif.), anti-α-COP, anti-β-COP, anti-β′-COP (Affinity BioReagents, Inc., Golden Colo.), goat anti-rabbit HRP, goat anti-mouse HRP (Zymed Laboratories, Inc., South San Francisco, Calif.).

In Vitro Nuclear Disassembly Assay and Microscopy

Demembranated condensed sperm chromatin was isolated from Xenopus testes as described previously (Powers, et al., In Current Protocols in Cell Biology (New York, John Wiley & Sons), pp. 11.10.11-11.11.24, (2001). For the assembly of nuclei, an ATP-generating system was added to 28 μl of crude egg extract. Sperm chromatin was added and nuclei were allowed to assemble by incubation at room temperature. After assembly for 1 hr, import substrate (NLS-HSA-RITC, prepared as in (Powers, et al., In Current Protocols in Cell Biology (New York, John Wiley & Sons), pp. 11.10.11-11.11.24, (2001)) was added to the reaction and incubated for another 30 min to assess the presence of functional pores and membrane integrity. Recombinant cyclin B with a deletion that confers stability was then added to the nuclei to initiate mitotic events (Murray, A. W., et al., Nature 339, 280-286 (1989). The role of cyclin synthesis and degradation in the control of maturation promoting factor activity Nature 339, 280-286, 1989). Samples were taken from the reaction at the indicated time points to monitor by fluorescence microscopy using DHCC (Calbiochem, San Diego, Calif.) to monitor membranes and Hoechst 33258 (Calbiochem) to monitor DNA. To examine the effects of protein fragments and antibodies on nuclear disassembly, the reagent in question was incubated with crude extract for 15 minutes at room temperature prior to the addition of sperm chromatin and energy mix. As noted, for protein fragments either 3 μg (corresponding to final concentrations of 3.3 μM, 1.7 μM, and 1.5 μM for GST, GST-N, and GST-Z respectively) or 4 μg (corresponding to final concentrations of 4.4 μM and 2 μM for GST and GST-Z respectively) was added. For antibody studies, 2.5 μg of antibodies against Nup153 (and corresponding pre-immune antibodies) and 5 μg of anti-β-COP (or pre-imunune) was used. In order to assess the effect of ADP-ribosylation factor 1 (ARF1) on nuclear disassembly, an inhibitory peptide of ARF1, GNMFANLFKGLFGKKE (SEQ ID NO: 28, ARF 2-17, represents amino acids residue 2-17 of Xenopus ARF1, Accession #AAA74582) was synthesized (Peptide Core Facility, University of Utah). Another peptide with the reverse sequence, EKKGFLGKFLNAF MNG (ARF 17-2, SEQ ID NO: 29), was synthesized in addition and used as a control. Purities of both peptides were above 95%. These peptides (at 11 or 22 μM as indicated) were also added 15 minutes prior to initiation of nuclear assembly. Brefeldin A (Calbiochem) was added to reactions at a final concentration of 0.6 μg/ml.

Images were acquired on a Zeiss Axioskop 2 (Carl Zeiss, Inc., Thornwood, N.Y.) using FV12, a 12 bit monochrome digital camera, and Olympus MicroSuite software (Olympus, Melville, N.Y.). Magnification, exposure, and processing were identical across samples in the same experiment. For quantitation, two samples were taken for the interphase time point (immediately prior to cyclin addition) and mitotic time-point (75 minutes after addition of cyclin). All of the intact nuclei in each sample were counted and the average of the two samples from the second time point was divided by the average of the two samples from the first time point. This number (x.p. 21 100) represents the relative number of nuclei that are present after an individual sample was shifted from interphase to mitosis.

Immunofluorescence

To assess the presence of β-COP, in vitro assembled nuclei were processed for immunofluorescence as described by Macaulay and Forbes (1996). Secondary antibodies used were Alexa Fluoro 488 goat anti-mouse IgG and Alexa Fluoro 568 goat anti-rabbit IgG (Molecular Probes, Eugene, Oreg.). Images were acquired as described above. To assess the status of the nuclear lamina, aliquots of the assembly/disassembly reaction were placed between siliconized coverslips and a slide. The slides were inserted into liquid nitrogen until bubbling ceased and the coverslips immediately removed. The frozen samples were then fixed and dehydrated in methanol for 1 hr at room temperature. Following the blocking and antibody incubation steps, the samples were mounted in 50% glycerol containing 2 mg/ml phenylenediamine and imaged using an Olympus FVX IX70 confocal microscope.

HeLa cells plated on coverslips were synchronized by incubating in media containing 2 mM thymidine (Sigma). After 12 hours, the thymidine was washed out and 11 hours later the samples were processed for indirect immunofluorescence microscopy as previously described (Griffis, E. R., et al., Mol Biol Cell. 13:1282-97 (2002)). This time point was chosen by monitoring cells under these conditions for progression into mitosis and selecting a time at which the culture was enriched for cells in prophase. Antibodies were as follows: anti-cyclin B1 monoclonal antibody at 1:500 (BD Biosciences), mAb414 at 1:2000 (Covance), and anti-β-COP at 1:1000 (Affinity BioRegents). Secondary antibodies and microscopy were as described above.

### Example 2

### Using the Nup153 Zinc finger Domain to Select for Peptides that Inhibit Nuclear Envelope Breakdown

To find peptide inhibitors of Nup153-COPI association, a phage display strategy was employed. A PhD phage display kit was purchased from New England Biolabs for this purpose. In this library, peptides with random arrays of 7 amino acids, flanked by two cysteines to constrain the peptide conformation, were encoded in the gene for the phage outer coat protein. Phage bearing these altered coat proteins were incubated with immobilized GST-ZnF protein or a distinct GST fusion protein (GST-RBD) as a control. Fifty microliters of glutathione beads (GT) were equilibrated and then bound to 300-500 ng of either GST-RBD or GST-hZnF. Ten microliters of the PhD phage library (1×10¹¹ pfu) were added to each of the bound RBD and hZnF tubes. After extensive washing (15×) with TBST (0.1% Triton), the bound phage were eluted with glycine and then amplified by adding to ER2537 bacteria and growing together for 4.5 hrs at 37 degrees. Bacteria were then pelleted and phage particles are precipitated from the supernatant with PEG overnight. These phage were then titered and the whole process repeated two more times (the latter two rounds were washed (25×) with TBS-T (0.5% Triton) or with RBD assay buffer). Ten individual plaques from each of the wash conditions were then picked for PCR amplification and sequencing.

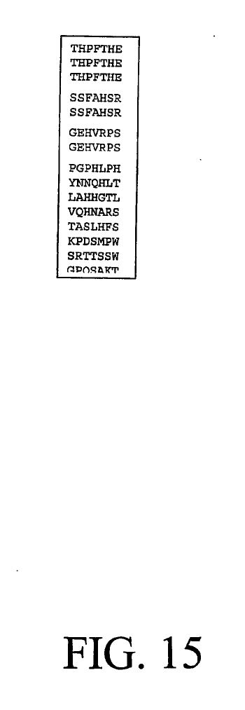

Results of the phage display screen are summarized in FIG. 15. Peptides chosen for further testing are boxed. The two shades of text indicate sequences derived from two different wash conditions in the third round of selection. Basic residues (H, R, and K) are also highlighted.

Synthetic peptides (13-mers containing the flanking cysteines and an additional few amino acids found in the library context) were synthesized and tested for function in the nuclear disassembly assay. Peptides were incubated at a final concentration of 44 μM in the assembly reaction for 15 minutes prior to the addition of sperm chromatin. A sample was taken at 90 minutes to assess nuclear formation. Cyclin was then added to initiate mitotic signaling and samples were assessed 75 and 95 minutes post cyclin addition. One peptide selected from the ZnF screen was inhibitory (ZNFPHD1=CTHPFTHECGGGS (SEQ ID NO: 30). The second peptide did not have activity (CGEHVRPSCGGGS, SEQ ID NO: 31). Two peptides derived from the parallel phage display screen using GST-RBD as the affinity matrix were also not inhibitory (CNNTRSPYCGGGS, SEQ ID NO: 32, CEQTYRVCGGGS, SEQ ID NO: 33).

The functional assessment is qualitative and reproducible in independent tests. Also of note, the ZNFPHD 1 peptide was not inhibitory when added after nuclear assembly has taken place. This suggests that the zinc finger domain of Nup153 is not accessible for saturation with the peptide when the pore is assembled.

### REFERENCES

Acharya, U., Jacobs, R., Peters, J. M., Watson, N., Farquhar, M. G., and Malhotra, V. (1995). The formation of Golgi stacks from vesiculated Golgi membranes requires two distinct fusion events. Cell 82, 895-904.
Ait chison, J. D., and Rout, M. P. (2002). A tense time for the nuclear envelope. Cell 108, 301-304.
Beaudouin, J., Gerlich, D., Daigle, N., Eils, R., and Ellenberg, J. (2002). Nuclear envelope breakdown proceeds by microtubule-induced tearing of the lamina. Cell 108, 83-96.
Buendia, B., and Courvalin, J. C. (1997). Domain-specific disassembly and reassembly of nuclear membranes during mitosis. Exp Cell Res 230, 133-144.
Burke, B., and Ellenberg, J. (2002). Remodelling the walls of the nucleus. Nat Rev Mol Cell Biol 3, 487-497.
Collas, I., and Courvalin, J. C. (2000). Sorting nuclear membrane proteins at mitosis. Trends Cell Biol 10, 5-8.
Cotter, L. A., Goldberg, M. W., and Allen, T. D. (1998). Nuclear pore complex disassembly and nuclear envelope breakdown during mitosis may occur by both nuclear envelope vesicularisation and dispersion throughout the endoplasmic reticulum. Scanning 20, 250-251. p. 23
Daigle, N., Beaudouin, J., Hartnell, L., Imreh, G., Hallberg, E., Lippincott-Schwartz, J., and Ellenberg, J. (2001). Nuclear pore complexes form immobile networks and have a very low turnover in live mammalian cells. J Cell Biol 154, 71-.
Dimaano, C., Ball, J. R., Prunuske, A. J., and Ullman, K. S. (2001). RNA association defines a functionally conserved domain in the nuclear pore protein Nup153. J Biol Chem 276, 45349-45357.
Drummond, S., Ferrigno, P., Lyon, C., Murphy, J., Goldberg, M., Allen, T., Smythe, C., and Hutchison, C. J. (1999). Temporal differences in the appearance of NEP-B78 and an LBR-like protein during Xenopus nuclear envelope reassembly reflect the ordered recruitment of functionally discrete vesicle types. J Cell Biol 144, 225-240.
Ellenberg, J., Siggia, E. D., Moreira, J. E., Smith, C. L., Presley, J. F., Worman, H. J., and Lippincott-Schwartz, J. (1997). Nuclear membrane dynamics and reassembly in living cells: targeting of an inner nuclear membrane protein in interphase and mitosis. J Cell Biol 138, 1193-1206.
Ewald, A., Hofbauer, S., Dabauvalle, M. C., and Lourim, D. (1997). Preassembly of annulate lamellae in egg extracts inhibits nuclear pore complex formation, but not nuclear membrane assembly. Eur J Cell Biol 73, 259-269.
Fahrenkrog, B., Maco, B., Fager, A. M., Koser, J., Sauder, U., Ullman, K. S., and Aebi, U. (2002). Domain-specific antibodies reveal multiple-site topology of Nup153 within the nuclear pore complex. J Struct Biol 140, 254-267.
Featherstone, C., Griffiths, G., and Warren, G. (1985). Newly synthesized G protein of vesicular stomatitis virus is not transported to the Golgi complex in mitotic cells. J Cell Biol 101, 2036-2046.
Frangioni, J. V., and Neel, B. G. (1993). Solubilization and purification of enzymatically active glutathione S-transferase (PGEX) fusion proteins. Anal Biochem 210, 179-187.
Gant, T. M., and Wilson, K. L., Eur J Cell Biol 74, 10-19 (1997). ARF is not required for nuclear vesicle fusion or mitotic membrane disassembly in vitro: evidence for a non-ARF GTPase in fusion. Eur J Cell Biol 74, 10-19.
Gonczy, P. (2002). Nuclear envelope: torn apart at mitosis. Curr Biol 12, R242-244.
Hang, J., and Dasso, M. (2002). Association of the human SUMO-1 protease SENP2 with the nuclear pore. J Biol Chem 277, 19961-19966.
Griffis, E. R. Altan, N., Lippincott-Schwartz, J., Powers, M. A. (2002). Nup98 is a mobile nucleoporin with transcription-dependent dynamics. Mol Biol Cell. 13:1282
Hetzer, M., Meyer, H. H., Walther, T. C., Bilbao-Cortes, D., Warren, G., and Mattaj, I. W. (2001). Distinct AAA-ATPase p97 complexes function in discrete steps of nuclear assembly. Nat Cell Biol 3, 1086-1091.
Kahn, R. A., Randazzo, P., Serafni, T., Weiss, O., Rulka, C., Clark, J., Amherdt, M., Roller, P., Orci, L., and Rothman, J. E. (1992). The amino terminus of ADP-ribosylation factor (ARF) is a critical determinant of ARF activities and is a potent and specific inhibitor of protein transport. J Biol Chem 267, 13039-13046.
Kondo, H., Rabouille, C., Newman, R., Levine, T. P., Pappin, D., Freemont, P., and Warren, G. (1997). p47 is a cofactor for p97-mediated membrane fusion. Nature 388, 75-78. p. 24
Lenart, P., and Ellenberg, J. (2003). Nuclear envelope dynamics in oocytes: from germinal vesicle breakdown to mitosis. Curr Opin Cell Biol 15, 88-95.
Lenart, P., Rabut, G., Daigle, N., Hand, A. R., Terasaki, M., and Ellenberg, J. (2003). Nuclear envelope breakdown in starfish oocytes proceeds by partial NPC disassembly followed by a rapidly spreading fenestration of nuclear membranes. J Cell Biol 160, 1055-1068.
Meyer, H. H., Wang, Y., and Warren, G. (2002). Direct binding of ubiquitin conjugates by the mammalian p97 adaptor complexes, p47 and Ufdl-Np14. Embo J 21, 5645-5652.
Moroianu, J., Blobel, G., and Radu, A. (1997). RanGTP-mediated nuclear export of karyopherin alpha involves its interaction with the nucleoporin Nup153. Proc Natl Acad Sci USA 94, 9699-9704.
Murray, A. W., Solomon, M. J., and Kirschner, M. W. (1989). The role of cyclin synthesis and degradation in the control of maturation promoting factor activity. Nature 339, 280-286.
Nakielny, S., Shaikh, S., Burke, B., and Dreyfuss, G. (1999). Nup153 is an M9-containing mobile nucleoporin with a novel Ran-binding domain. Embo J 18, 1982-1995.
Newport, J., and Spann, T. (1987). Disassembly of the nucleus in mitotic extracts: membrane vesicularizafion, lamin disassembly, and chromosome condensation are independent processes. Cell 48, 219-230.
Nickel, W., Brugger, B., and Wieland, F. T. (2002). Vesicular transport: the core machinery of COPI recruitment and budding. J Cell Sci 115, 3235-3240.
Pante, N., Bastos, R., McMorrow, I., Burke, B., and Aebi, U. (1994). Interactions and three-dimensional localization of a group of nuclear pore complex proteins. J Cell Biol 126, 603-617.
Powers, M. A., Evans, E. K., Yang, J., and Kombluth, S. (2001). Preparation and use of interphase Xenopus egg extracts. In Current Protocols in Cell Biology (New York, John Wiley & Sons), pp. 11.10.11-11.11.24.
Powers, M. A., Macaulay, C., Masiarz, F. R., and Forbes, D. J. (1995). Reconstituted nuclei depleted of a vertebrate GLFG nuclear pore protein, p97, import but are defective in nuclear growth and replication. J Cell Biol 128, 721-736.
Rabouille, C., Levine, T. P., Peters, J. M., and Warren, G. (1995). An NSF-like ATPase, p97, and NSF mediate cisternal regrowth from mitotic Golgi fragments. Cell 82, 905-914.
Rossanese, O. W., and Glick, B. S. (2001). Deconstucting Golgi inheritance. Traffic 2, 589-596.
Ryan, K. J., and Wente, S. R. (2002). Isolation and characterization of new Saccharomyces cerevisiae mutants perturbed in nuclear pore complex assembly. BMC Genet 3, 17.
Salina, D., Bodoor, K, Eckley, D. M., Schroer, T. A., Rattner, J. B., and Burke, B. (2002). Cytoplasmic dynein as a facilitator of nuclear envelope breakdown. Cell 108, 97-107.
Sasagawa, S., Yamamoto, A., Ichirnura, T., Omata, S., and Horigome, T. (1999). In vitro nuclear assembly with affinity-purified nuclear envelope precursor vesicle fractions, PV1 and PV2. Eur J Cell Biol 78, 593-600.
Shah, S., and Forbes, D. J. (1998). Separate nuclear import pathways converge on the nucleoporin Nup153 and can be dissected with dominant-negative inhibitors. Curr Biol 8, 1376-1386.
Shah, S., Tugendreich, S., and Forbes, D. (1998). Major binding sites for the nuclear import receptor are the internal nucleoporin Nup153 and the adjacent nuclear filament protein Tpr. J Cell Biol 141, 31-49.
Smythe, C., Jenkins, H. E., and Hutchison, C. J. (2000). Incorporation of the nuclear pore basket protein nup153 into nuclear pore structures is dependent upon lamina assembly: evidence from cell-free extracts of Xenopus eggs. Embo J 19, 3918-3931.
Sukegawa, J., and Blobel, G. (1993). A nuclear pore complex protein that contains zinc finger motifs, binds DNA, and faces the nucleoplasm. Cell 72, 29-38.
Suntharalingam, M. and Wente, S. R. (2003). Peering through the pore. Nuclear pore complex structure, assembly, and function. Dev. Cell 4:775-89.
Sutterlin, C., Hsu, P., Mallabiabarrena, A., Malhotra, V. (2002) Fragmentation and dispersal of the pericentriolar Golgi complex is required for entry into mitosis in mammalian cells. Cell 109:359-69.
Terasaki, M., Campagnola, P., Rolls, M. M., Stein, P. A., Ellenberg, J., Hinkle, B., and Slepchenko, B. (2001). A new model for nuclear envelope breakdown. Mol Biol Cell 12, 503-510.
Ullman, K. S., Shah, S., Powers, M. A., and Forbes, D. J. (1999). The nucleoporin nupl 53 plays a critical role in multiple types of nuclear export. Mol Biol Cell 10, 649-664.
Vasu, S., Shah, S., Orjalo, A., Park, M., Fischer, W. H., and Forbes, D. J. (2001). Novel vertebrate nucleoporins Nupl33 and Nupl60 play a role in mRNA export. J Cell Biol 155, 339-354.
Vasu, S. K., and Forbes, D. J. (2001). Nuclear pores and nuclear assembly. Curr Opin Cell Biol 13, 363-375.
Vigers, G. P., and Lohka, M. J. (1991). A distinct vesicle population targets membranes and pore complexes to the nuclear envelope in Xenopus eggs. J Cell Biol 112, 545-556.
Walther, T. C., Fomerod, M., Pickersgill, H., Goldberg, M., Allen, T. D., and Mattaj, I. W. (2001). The nucleoporin Nup153 is required for nuclear pore basket formation, nuclear pore complex anchoring and import of a subset of nuclear proteins. Embo J 20,5703-5714.
Wang, B., Alam, S. L., Meyer, H. H., Payne, M., Stemmler, T. L., Davis, D. R., and Sundquist, W. L (2003). Structure and Ubiquitin interactions of the conserved NZF domain of Np14. J Biol. Chem.
Warren, G., and Shorter, J. (2002). Golgi Architecture and Inheritance. Annu Rev Cell Dev Biol.
Yang, L., Guan, T., and Gerace, L. (1997). Integral membrane proteins of the nuclear envelope are dispersed throughout the endoplasmic reticulum during mitosis. J Cell Biol 137, 1199-1210.
Zaal, K. J., Smith, C. L., Polishchuk, R. S., Altan, N., Cole, N. B., Ellenberg, J., Hirschberg, K., Presley, J. F., Roberts, T. H., Siggia, E., et al. (1999). Golgi membranes are absorbed into and reemerge from the ER during mitosis. Cell 99, 589-601.
Zhang, H., Saitoh, H., and Matunis, M. J. (2002). Enzymes of the SUMO modification pathway localize to filaments of the nuclear pore complex. Mol Cell Biol 22, 6498-6508.

1. A composition comprising a molecule that inhibits nuclear envelope breakdown.

2. The composition of claim 1, wherein the molecule is not nocodozole, p50/dynamitin, or p62.

3. The composition of claim 1, wherein the molecule does not arrest cell cycle prior to nuclear envelope breakdown.

4. (canceled)

5. (canceled)

6. (canceled)

7. (canceled)

8. (canceled)

9. (canceled)

10. (canceled)

11. The composition of claim 1, wherein the composition binds a peptide, wherein the peptide comprises a sequence having at least 47% identity to amino acids 658 to 891 of SEQ ID NO: 2.

12. (canceled)

13. (canceled)

14. The composition of claim 1, wherein the molecule is an antibody.

15. (canceled)

16. (canceled)

17. (canceled)

18. (canceled)

19. (canceled)

20. (canceled)

21. (canceled)

22. (canceled)

23. (canceled)

24. The composition of claim 1, wherein the molecule comprises a peptide such as a protein variant, a chimeric protein or a related derivative.

25. The composition of claim 1, wherein the molecule is a small molecule.

26. The composition of claim 25, wherein the small molecule comprises Brefeldin A.

27. The composition of claim 1, wherein the molecule is an aptamer.

28. A method of identifying a compound that inhibits nuclear envelope breakdown, comprising adding the compound to a system wherein the system comprises Nup153 and COPI, wherein Nup153 and COPI can form a complex, and assaying for a molecule that decreases the amount of complex formed compared to the amount of complex formed in the absence of the compound

29. (canceled)

30. (canceled)

31. (canceled)

32. (canceled)

33. (canceled)

34. A method of identifying and producing a compound, the method comprising
bringing into contact a test compound and Nup153;
assessing the activity of Nup153;
comparing the activity of Nup153 when exposed to the test compound to activity of Nup153 in the absence of the test compound;
wherein inhibition of Nup153 when exposed to the test compound identifies the test compound;
and producing the identified test compound.

35. (canceled)

36. (canceled)

37. (canceled)

38. (canceled)

39. (canceled)

40. A method of identifying and producing a compound, the method comprising
bringing into contact a test compound and Nup358/RanBP2;
assessing the activity of Nup358/RanBP2;
comparing the activity of Nup358/RanBP2 3 when exposed to the test compound to activity of Nup153 in the absence of the test compound;
wherein inhibition of Nup 153 when exposed to the test compound identifies the test compound;
and producing the identified test compound.

41. (canceled)

42. (canceled)

43. (canceled)

44. (canceled)

45. (canceled)

46. A method of producing an antibody comprising, administering a region of Nup153 to an animal.

47. (canceled)

48. (canceled)

49. A method of using the antibody of claim 46, the method comprising administering the antibody to an animal.

50. A method of inhibiting a cell cycle of a cell comprising administering a Nup153 inhibitor to the cell.

51. A method of identifying proteins that interact with Nup153 comprising operably linking Nup153 or a fragment of Nup153 to a DNA binding domain forming a first nucleic acid, transfecting a cell with the first nucleic acid, wherein the cell comprises a protein or protein fragment which is operably linked to an transcription activation domain, wherein the cell comprises a reporter system specific for the DNA binding domain, assaying the amount of expression from the reporter system, wherein an increase in expression indicates an interaction between the Nup153 or Nup153 fragment and the protein or protein fragment.

52. (canceled)

53. A system for assaying nuclear breakdown comprising Nup153, further comprising COPI, and further comprising a Xenopus laevis egg extract.

54. (canceled)

55. (canceled)

56. (canceled)

57. A method of evaluating expression of Nup153, comprising
contacting cells undergoing mitosis with a probe for Nup153;
detecting expression of Nup153.

58. (canceled)

59. A method of inhibiting nuclear envelope breakdown, comprising contacting a cell with the compound from claim 1.

60. (canceled)

61. (canceled)

62. A method of treating a subject with cancer, comprising administering to the subject an effective amount of a compound that inhibits nuclear envelope breakdown.

63. (canceled)


SEQUENCE LISTING (35 sequences)

SEQ ID NO: 1 - DNA, 5687 bp, Artificial Sequence
  Description of Artificial Sequence/note = synthetic construct
ttccccgctgccagcactcaccctctctccgctcctgctcgcaacctcgcggttcctggggtgcttgtgcccactgtgtggacagcgcgggcggacttttgggccggggccgggcggcgggggaggctctctaaggcctccgcctctgcctctcccgcccccttacccgccccggagcgggaagcggcggaggctccgccatggcctcgggagccggaggagtcggagggggcggtggcggcaagatccggacgcggcgttgccaccaggggccaattaagccttaccagcaggggcgacaacagcatcagggcattcttagcagggttacagaatctgttaagaatattgtgccagggtggctacaaagatacttcaacaagaatgaagatgtatgcagctgttcaacagacacaagcgaggttccacgctggccagaaaataaagaggaccatctggtatatgccgatgaggagagctctaatattactgatgggagaatcacacctgagccagcagtcagtaatacagaagaaccttcaacaactagtactgcttcaaattatccagatgtgttaacaaggccttctcttcatcggagccatctgaatttttccatgttggaatcccctgcattacactgtcagccatctacatcctcggcattcccaattggcagttcgggattttcccttgtaaaggaaattaaagattctacctctcagcatgatgatgataacatctcaactaccagtggtttttcttcaagagcttctgataaagatataactgtttcaaagaacacttcattgccacctctgtggtccccagaagctgaacgttctcactcactctcacagcacactgccaccagctcaaaaaaaccagcattcaacttgtctgcctttggaacactttccccttcacttgggaattcttcaatccttaaaaccagtcagcttggagattctcctttttatcctggaaaaacaacatacggtggggcagcagctgctgtaagacagtctaaactacgaaatacaccttatcaggcaccagttagaagacaaatgaaagctaagcaactcagtgcacaatcttacggtgtgaccagttcaacagctcggcgaatattgcagtctttagagaagatgtcaagccctttagcggatgcaaaaagaattccatccattgtttcttctcctctgaattctcctcttgataggagtgggatagatatcacagattttcaggccaaaagagaaaaggtggattctcaatatcctcctgttcagagacttatgaccccaaagccagtttccatagcaacaaatcgaagtgtttattttaaaccatctctgactccttctggtgaattcaggaagactaatcaaagaatagataacaagtgcagtactggatatgaaaaaaatatgacacccggacaaaatagagaacaacgagaaagtggcttttcatatccaaatttcagtttgcctgcagccaatggtttatcttctggagtaggtggtggaggtggcaagatgagacgagaaagaacacgctttgttgcttctaaacctctggaggaggaggaaatggaagttccagtattaccgaaaatctctctaccgatcaccagttcttcactgcctacctttaattttagttcccctgagatcacaacttcctctccatcacccatcaattcgtctcaagcattaacaaacaaggtacaaatgacctctccgagcagcactggcagtcccatgtttaaattttcatctccaatcgtaaaatctactgaggcaaatgtactacctccatcatctattggatttacatttagtgtgcctgttgcaaaaacagcagaactttctggttctagtagtactttagaaccaattataagtagttcagctcatcatgtcactacagtgaacagtacaaattgtaagaagacaccacctgaagattgtgagggtccttttagacctgcagaaatcctgaaagaaggaagtgttctagatattctgaaaagccctggtttcgcatcgccgaagatagattctgttgctgctcagcccaccgcaacaagcccagtagtttatacaagaccagcaataagtagcttttcttctagtggaattgggtttggggagagtttaaaagctgggtcatcatggcagtgtgatacatgtctactccagaacaaagttacagacaacaaatgcatagcctgtcaagcagcaaaattgtcacccagagatactgctaaacagactggaattgaaacaccaaataaaagtggcaaaacaactctttctgcatcagggacaggctttggagacaaatttaaaccagtgataggcacttgggattgtgatacctgtttagtgcaaaataaacctgaagcaataaaatgtgtagcctgtgaaacaccgaaacctggaacttgtgtgaagcgagcccttacattgacagtggtttcggaaagtgctgagactatgactgcttcatcttccagctgcactgtaaccactggtaccttaggatttggagataaattcaaaaggcccattggatcttgggagtgttcagtatgctgtgtttctaataatgcagaagacaataagtgtgtgtcctgtatgtctgagaaaccaggaagttcagtacctgcttcaagtagcagcactgtacctgtctctctgccttctggaggctctctaggattggaaaagttcaagaaacccgagggaagctgggactgtgaattgtgcctagtgcagaataaggcagactctaccaaatgtttggcatgtgaaagtgcaaagccaggcacaaaatctgggtttaaaggctttgacacatcttcctcatcttcgaactcagcagcctcctcatccttcaaatttggtgtctcatcatcctcttctgggccttctcagactttaacaagcactggaaattttaaatttggagatcagggaggattcaaaataggtgtgtcatctgattctgggtctataaaccccatgagtgaaggctttaaattttctaaaccaataggagattttaaatttggagtttcatctgaatctaagcccgaagaagttaaaaaagatagtaagaatgataattttaagtttggactttcttctggtttaagcaacccagtttctttaactccatttcaatttggggtatctaatcttggacaggaagaaaagaaagaggaactgcccaaatcttcctctgcaggttttagctttggtacaggtgttattaactccacccctgctcctgctaacaccatagtgacctctgagaacaagagcagcttcaaccttggaaccatagaaaccaagagtgcttcagtggctcctttcacatgtaagacatcagaagctaaaaaagaagaaatgcctgccaccaaaggaggattctcttttggcaacgtggagcctgcctctctgccatctgcctcagtgtttgttttgggaaggacagaagagaaacagcaagagcctgtcacttctacttccctagtttttgggaagaaagctgacaatgaagagccaaagtgtcaaccagtgttttcctttgggaattcagagcaaaccaaagatgagaattcttcaaagtccacatttagttttagtatgacaaaaccatctgagaaggaatctgaacagccagcaaaagccacttttgcctttggagctcaaactagtactacagctgatcaaggtgcagcaaagccagtttttagtttcttgaacaacagttcctctagttcaagtacaccagccacttctgctggtggtggcatatttggtagttccacctcttcctccaatccacctgtggctacctttgtgtttggacagtccagcaatcctgtgagcagctctgcctttggtaacactgctgaatccagcacctctcagtctttgctattttctcaagatagcaaactagcaaccacatccagcacaggtacagctgtcaccccatttgtctttggtccaggagccagcagtaataatactaccacctctggtttcggctttggagccacaaccacatctagctctgcaggatcctcctttgtatttggaactggaccctcagcaccatctgccagtccagcatttggtgctaaccagaccccaacatttggacaaagtcaaggtgccagccagcccaatcccccaggctttggatctatatcatcttccacagcattatttcccactggttctcagcctgcaccacctacttttgggacagtgtcaagcagtagccagccccctgtgtttggacagcaacctagtcagtctgcatttggctctggaacaactcctaattctagttcggctttccagtttggcagcagcactacaaatttcaacttcacaaacaacagtccatcaggagtgttcacatttggtgcaaattctagcacacctgcagcctcagcccagccttcaggctcggggggctttccatttaaccagtctccagcagcatttacagtggggtcaaatgggaaaaatgtgttctcttcttctggaacttcattctctggtcgcaagataaagactgctgttagacgcaggaaataaaggtcacattggtgttgtactcaattttaacaacagctggtgccctgctttcagatactggattgtactttgtgctggggttatctgaagtcagatctgcctaaggacttctttaattttggaattttcctcctttctctttcgttacagaagccccaccctgcctcacccacccttttttaaataaataaatagctagactggtgactgattcttcagcaaaaatattttatgatccagcagattattcactgatttgacatagtctggctgtacccaggaatggagcctgcacggtgaatggctttgtatagaacctctttgtctacaccattatgtgcgctgataagcgttcatggaacgcgttgaaattgtaattatatctgaggaattctgtatagattagaattctgtatagattagagagtgttgaaacggatgatttctatgctgagtttgtgctggtgtatgtgtgaagtgagtgagttgggtgtattgtgcgctaaacttttctgatagaggaagcctgattaaagaatggtccgtgctaaggacttgttagatctagttcactctccatttaataattatatgctatttctatattttcattctcctatcacctgtcttgcctttttcattattttattatgaaacttgtgtaaatacaattttgtttctgtactttttggcataacataaatctgtgaacttgaaatttgaattttgtgttagagatttttttgttgtttgtttagtcttgtctcagattttattatgtaaatcccattattcaaagttgcctaaatccatttggaaatctttaaaaaaaaaattggggattcttaaagttgaatttattggcttttctgatccagttttgtttggaccaaaaaccagtattgtacaaagtattaagcatatatttttatatttactaaaatggactgtggtgactttggataataaggaaaagtttaatattaaagccatgtttattacagtataattaacatgttaaaccatgggataaatgccatcaataaaaaattatgacat

SEQ ID NO: 2 - protein, 1475 aa, Artificial Sequence
  Description of Artificial Sequence/note = synthetic construct
MASGAGGVGGGGGGKIRTRRCHQGPIKPYQQGRQQHQGILSRVTESVKNIVPGWLQRYFNKNEDVCSCSTDTSEVPRWPENKEDHLVYADEESSNITDGRITPEPAVSNTEEPSTTSTASNYPDVLTRPSLHRSHLNFSMLESPALHCQPSTSSAFPIGSSGFSLVKEIKDSTSQHDDDNISTTSGFSSRASDKDITVSKNTSLPPLWSPEAERSHSLSQHTATSSKKPAFNLSAFGTLSPSLGNSSILKTSQLGDSPFYPGKTTYGGAAAAVRQSKLRNTPYQAPVRRQMKAKQLSAQSYGVTSSTARRILQSLEKMSSPLADAKRIPSIVSSPLNSPLDRSGIDITDFQAKREKVDSQYPPVQRLMTPKPVSIATNRSVYFKPSLTPSGEFRKTNQRIDNKCSTGYEKNMTPGQNREQRESGFSYPNFSLPAANGLSSGVGGGGGKMRRERTRFVASKPLEEEEMEVPVLPKISLPITSSSLPTFNFSSPEITTSSPSPINSSQALTNKVQMTSPSSTGSPMFKFSSPIVKSTEANVLPPSSIGFTFSVPVAKTAELSGSSSTLEPIISSSAHHVTTVNSTNCKKTPPEDCEGPFRPAEILKEGSVLDILKSPGFASPKIDSVAAQPTATSPVVYTRPAISSFSSSGIGFGESLKAGSSWQCDTCLLQNKVTDNKCIACQAAKLSPRDTAKQTGIETPNKSGKTTLSASGTGFGDKFKPVIGTWDCDTCLVQNKPEAIKCVACETPKPGTCVKRALTLTVVSESAETMTASSSSCTVTTGTLGFGDKFKRPIGSWECSVCCVSNNAEDNKCVSCMSEKPGSSVPASSSSTVPVSLPSGGSLGLEKFKKPEGSWDCELCLVQNKADSTKCLACESAKPGTKSGFKGFDTSSSSSNSAASSSFKFGVSSSSSGPSQTLTSTGNFKFGDQGGFKIGVSSDSGSINPMSEGFKFSKPIGDFKFGVSSESKPEEVKKDSKNDNFKFGLSSGLSNPVSLTPFQFGVSNLGQEEKKEELPKSSSAGFSFGTGVINSTPAPANTIVTSENKSSFNLGTIETKSASVAPFTCKTSEAKKEEMPATKGGFSFGNVEPASLPSASVFVLGRTEEKQQEPVTSTSLVFGKKADNEEPKCQPVFSFGNSEQTKDENSSKSTFSFSMTKPSEKESEQPAKATFAFGAQTSTTADQGAAKPVFSFLNNSSSSSSTPATSAGGGIFGSSTSSSNPPVATFVFGQSSNPVSSSAFGNTAESSTSQSLLFSQDSKLATTSSTGTAVTPFVFGPGASSNNTTTSGFGFGATTTSSSAGSSFVFGTGPSAPSASPAFGANQTPTFGQSQGASQPNPPGFGSISSSTALFPTGSQPAPPTFGTVSSSSQPPVFGQQPSQSAFGSGTTPNSSSAFQFGSSTTNFNFTNNSPSGVFTFGANSSTPAASAQPSGSGGFPFNQSPAAFTVGSNGKNVFSSSGTSFSGRKIKTAVRRRK

SEQ ID NO: 3 - DNA, 1200 bp, Artificial Sequence
  Description of Artificial Sequence/note = synthetic construct
atggcggcagccggaggaggaggccctggtggtccaggaaccgggggcaaaatacgcagtaggagatatcacttgagttccggcaggactccgtacagcaagagccggcagcggcagcagggtatcatcagccgtgtcacggatactgtcaagagcattgttcctggatggttgcagaagtattttaacaaacaagaagaggaacatgaccgagtacatagtgcaagtgaagtaattgtgaatgatactgaggctagagaaaataatgctcaacaccatatatatgatgatgatgatgaagaaggaaattctcccactgatggcagagtcactcctgagccagttattaatgttgatgaagaagttccgtccactagtcagtcagcaataaacaacacagatgcccttactaggccatctttacaccgtgctagtctaaacttcaacatatttgactctcctgcactgaactgccagccttcaacctcttcggcatttcctattggcacctctggcttctcacttataaaagagatcaaggactctacctcgcaacatgatgacgataacatctccaccaccagtggctttacctctagagcatctgataaggatctagccgtttcaaaaaatgtcagcgtgccacctctttggtctcctgaagttgatcgctcacagtctctgtcacataactcctccatgacctccaaaaaaccaacttttaatctttctgctttcggttctctctcaccttctcttggaaacgcatctatcttaaaccgtcagcttggtgattctcctttttatcctggcaaaacaacataccaaggtgcagctgcagtccgttcctcgcgagtgcgcgccaccccatatcaagcacctttacgaagacaggtgaaggcaaaaccagcagcacactctcagcagtgtggtgttaccagctccgctgcacgccgcattctacagtctttggagaagatgtcaagtcctttagcggatgccaaaaggatcccatcaaactcttctttatcacatacaccagaaaagaacgtcatggatattcccgaaaacccatcaaaaaggaaaaaggtggagtccccatttcctccagttcagaggctcgtgactccaaaatccatttctgtctctgcaaaccgttccctgtatatcaagccttcactgacaccatctgctgtatca

SEQ ID NO: 4 - protein, 400 aa, Artificial Sequence
  Description of Artificial Sequence/note = synthetic construct
MAAAGGGGPGGPGTGGKIRSRRYHLSSGRTPYSKSRQRQQGIISRVTDTVKSIVPGWLQKYFNKQEEEHDRVHSASEVIVNDTEARENNAQHHIYDDDDEEGNSPTDGRVTPEPVINVDEEVPSTSQSAINNTDALTRPSLHRASLNFNIFDSPALNCQPSTSSAFPIGTSGFSLIKEIKDSTSQHDDDNISTTSGFTSRASDKDLAVSKNVSVPPLWSPEVDRSQSLSHNSSMTSKKPTFNLSAFGSLSPSLGNASILNRQLGDSPFYPGKTTYQGAAAVRSSRVRATPYQAPLRRQVKAKPAAHSQQCGVTSSAARRILQSLEKMSSPLADAKRIPSNSSLSHTPEKNVMDIPENPSKRKKVESPFPPVQRLVTPKSISVSANRSLYIKPSLTPSAVS

SEQ ID NO: 5 - DNA, 4305 bp, Artificial Sequence
  Description of Artificial Sequence/note = synthetic construct
cgcaaaccgttccctgtatatcaagccttcactgacaccatctgctgtatcaaatacaaacagtcgcagaatacagccagacaagcacaatgagtccacaaagaataatctgcaatcaacatctcagtctcacagcttctcttacccaaagttcagtactccggcatccaatggattgtcatcaggaacaggtggtggcaaaatgatgagggaaaaaggctctcattactcaacaaagccagctaatgaggagctggatgggcctgttttacctgaaatcccattgcctcttagcacagcagcccttcccagctttcaattttcaactttgtctggctctgctacttcgcctatctctgtcactaaacctgcaaacagtacgacatgtcacttaaccagcagtagtccatcgttcacattttcgtcccctattgtaaaatcgactgaatcaaatgctcagttttctggctcatctgttgatttcactttcagtgtaccagcagcgaaagcgtcatctgcaacatctgatgagagcaaggtgtctgctgtaagtagcgcggctaaaacacatgctgctgttaatagcagtgcaaagaacactgatgatgaacaagtgggattctgtaaacctgcaaaaactctgaaggaagggagtgtgttagacatgctgagaagtccagggttttcttctttaccctccctgctaacttcagaatcaactcttaacagaagcacaccaaccctctcaaagactgtggggaatacattctcccctgcaaatgtatcattaggagtaggaagcaagcaatttggactttggcaatgcagcgcatgctttcatgagaatatgtcatctgatagcaactgtatttcttgctctgctgtaaaacctcggcctacagaaacctctaagaaactacctgcttcaccaccaagtagcaatacaaaaagcactgtcccactttctagcacaccaggactaggagacatatttaaaaaacctgcaggcatgtgggattgtgacacttgtttagtacagaacaaagcagaagtaacaaaatgtgttgcttgtgaaacccccaaacctgggacttgtataaaagctaccttgttgataccgtccaccacaaagtctataaatccagccacaaatacccttgcttttgcatcttgctctgcaagcattccaaatgaagaaatgttcaaaaagcctatgggatcttgggagtgcacagtttgtcatatgcagaataaaacagaggataatacatgtgtgggctgcaaagctgagaaaccaggaaccgtcaagagcgtacctactgctgcaccatcagggctgctgggattgttacatcagtttaaaaaaccaacagggagctgggattgtgatgtctgcttaatacaaaacaaaccagaagccgccaaatgtattgcatgtgaaagtgccaaaccaggcaccaaagcagaaccgaaaggtacttttgatacagtgaaaaattccgtttcagttgcacccctttcttcagggcagctgggattgttagatcagtttaagaagtcggcaggaagctgggattgtgatgtctgcctagtagaaaacaaaccagaagccacaaaatgtgttgcctgtgagacctcaaaaccaggcacaaaagcagaattaaaaggttttggaacatcaaccttttcctctggaacagcagctccgaccttcaagtttggtgtgcagtcttcagatagcactgctgagttaaaatcgggtgcctctacaagtggttttgccaaatctataggggattttaaatttggtttagtgtctgcatccacaacaacagaggaaactggcaagaaaagctttacatttggctcttctactaccaatgaagtttcagcaggttttaagtttggcattgccggttctgctcagacaaaaccagacaccctaagtcagtctaccacaagtggtttcacatttggctctgtttccaacacggtttctcttgctcctgctgcaactagctcatcgagtacaggcttgcaagtggcggctgcaattgctgattccaaccttgcaactactgctgccttgaaatcagcagaagagaaaaaggccgaagcacctacaataacaccgttttcttttgggaaaacagatcaaaataaagagactgcatccacctcttttgtctttggaaagaaagatgaaaagacagactctgctccaactggatcttcctttgcttttgggctaaaaaaggatggggaggaatctaagccgtttctttttggaaaacctgagccaaccaaagtagatggcaatgcagcatctgctggttttgcttttggtgtgactaatcccacagagaaaaaggatattgaacagcctggaaaatcagtttttgccttcggtgctcaaacttcaatcacagatgctggagcttccaagcaaccttttagttttctgaccaatgtctcctccactgcagcttcatcaagcacttgtggtgtttccagcagtgtatttggcagtgtgactcagtcaagcactcctgcaacccctagtaatgtgtttggcagtgctatttcagcaaatgctcctgccccgtctagtggtgtatttggaaacctcactccatcaaatgcccctgcagcttctagcactttgtttggtaacgttgctccctcaagtactccttctggctccagtggtttgtttggcactgcagctgcatccagtactcctgccacttccactagcttgtttggctctgcagctaaatcgagtgctcctgcaagctctggaggggtgttcaacagtgcagctcctgctgccccagcatcaactacaagtagtgtgtttggcagtgtagcctcttcaacaaacacttctgcaaattctgcaaacatatttggaagctctggtggagcggccactgctcctggggcttttgtgtttggacagccagctagtactgctagcactgtttttggaaactcgagcgaatcaaaatcgacctttgttttctctggtcaggaaaataaacccgtgacctctgcaagtacctctgtgactccatttctatttggagcagtgtctgccactactaccccagcagctccaagttttaactttggacgaacaatcacttcaaatacaacaggcactagctcgtctccatttatctttggtgctggtgcaagtggctctgcttcttcaagtataacggctcaggccaatccagtgccagcatttgggcaaagttctaatccgtctactgcacccgcttttggctcttcaacttcagttccagtgtttccagctgggagttctcagcaagtcccggcctttggttcgagtagtgcacaaccccctgtgtttgggcaacaagccgctcagccttcttttggatctcctgctgcacccagtgctggatctggtttcccgtttggcaacaacgcaaacttcaactttaacagcacaaattcatcaggtggagtcttcacgtttaatgcaaattcaggatccaccacccaaccacctcccccaggatacatgtttaatgcagctgcgccaggttttaacataggaacgaatggcaggactactccagcatctactatttctactcgcaagataaagaccgccagaagaagaaagtaattgaatgaatgtactggaagtaatggttgtttcaagaaatgaatggtgtccctgcattgtgcaaatactggagtgttcctcatgctgtgtgtagtggaagtcagatcagcccaaggacaaaaatatatgctgaaacttgtctctttttcctctgtaattaaaacatggtgatgggaaatttcttaaccaagaaatctgattccggcgcctcctttgtttacctgacttggcatgatttggctatacgagtgtttatagcctgcacagcgaatggcttttgtataatacctctttcatctgcaccactattttattgatgctttgaaattgtaattgtgagtgaggagccgtgtataggttacagactgatttctacaccacagttgtgctggtgaatgtgtggagcggagtgtagtgtgcaccgaaacttttctgcttacggaagactgactcatgaatggtccttacataggatgtgctagatctgtacttctctttcaaatatatgccttttcttctccttctcctcccccctgttttcattcagctcttgtcatcttctccattattttattatattcatgtaaatacatttgtttctccatgtacctttcagcataaactaaatcaaaaaaaaaaaaaaa

SEQ ID NO: 6 - protein, 1219 aa, Artificial Sequence
  Description of Artificial Sequence/note = synthetic construct
ANRSLYIKPSLTPSAVSNTNSRRIQPDKHNESTKNNLQSTSQSHSFSYPKFSTPASNGLSSGTGGGKMMREKGSHYSTKPANEELDGPVLPEIPLPLSTAALPSFQFSTLSGSATSPISVTKPANSTTCHLTSSSPSFTFSSPIVKSTESNAQFSGSSVDFTFSVPAAKASSATSDESKVSAVSSAAKTHAAVNSSAKNTDDEQVGFCKPAKTLKEGSVLDMLRSPGFSSLPSLLTSESTLNRSTPTLSKTVGNTFSPANVSLGVGSKQFGLWQCSACFHENMSSDSNCISCSAVKPRPTETSKKLPASPPSSNTKSTVPLSSTPGLGDIFKKPAGMWDCDTCLVQNKAEVTKCVACETPKPGTCIKATLLIPSTTKSINPATNTLAFASCSASIPNEEMFKKPMGSWECTVCHMQNKTEDNTCVGCKAEKPGTVKSVPTAAPSGLLGLLHQFKKPTGSWDCDVCLIQNKPEAAKCIACESAKPGTKAEPKGTFDTVKNSVSVAPLSSGQLGLLDQFKKSAGSWDCDVCLVENKPEATKCVACETSKPGTKAELKGFGTSTFSSGTAAPTFKFGVQSSDSTAELKSGASTSGFAKSIGDFKFGLVSASTTTEETGKKSFTFGSSTTNEVSAGFKFGIAGSAQTKPDTLSQSTTSGFTFGSVSNTVSLAPAATSSSSTGLQVAAAIADSNLATTAALKSAEEKKAEAPTITPFSFGKTDQNKETASTSFVFGKKDEKTDSAPTGSSFAFGLKKDGEESKPFLFGKPEPTKVDGNAASAGFAFGVTNPTEKKDIEQPGKSVFAFGAQTSITDAGASKQPFSFLTNVSSTAASSSTCGVSSSVFGSVTQSSTPATPSNVFGSAISANAPAPSSGVFGNLTPSNAPAASSTLFGNVAPSSTPSGSSGLFGTAAASSTPATSTSLFGSAAKSSAPASSGGVFNSAAPAAPASTTSSVFGSVASSTNTSANSANIFGSSGGAATAPGAFVFGQPASTASTVFGNSSESKSTFVFSGQENKPVTSASTSVTPFLFGAVSATTTPAAPSFNFGRTITSNTTGTSSSPFIFGAGASGSASSSITAQANPVPAFGQSSNPSTAPAFGSSTSVPVFPAGSSQQVPAFGSSSAQPPVFGQQAAQPSFGSPAAPSAGSGFPFGNNANFNFNSTNSSGGVFTFNANSGSTTQPPPPGYMFNAAAPGFNIGTNGRTTPASTISTRKIKTARRRK

SEQ ID NO: 7 - DNA, 10697 bp, Artificial Sequence
  Description of Artificial Sequence/note = synthetic construct
cacagtggtcctccgccggctacggcgctgcgtcactggtttgcaggcgctttcctcttggaagtggcgactgctgcgggcctgagcgctggtctcacgcgcctcgggagccaggttggcggcgcgatgaggcgcagcaaggctgacgtggagcggtacatcgcctcggtgcagggctccaccccgtcgcctcgacagaagtcaatgaaaggattctattttgcaaagctgtattatgaagctaaagaatatgatcttgctaaaaaatacatatgtacttacattaatgtgcaagagagggatcccaaagctcacagatttctgggtcttctttatgaattggaagaaaacacagacaaagccgttgaatgttacaggcgttcagtggaattaaacccaacacaaaaagatcttgtgttgaagattgcagaattgctttgtaaaaatgatgttactgatggaagagcaaaatactggcttgaaagagcagccaaacttttcccaggaagtcctgcaatttataaactaaaggaacagcttctagattgtgaaggtgaagatggatggaataaactttttgacttgattcagtcagaactttatgtaagacctgatgacgtccatgtgaacatccggctagtggaggtgtatcgctcaactaaaagattgaaggatgctgtggcccactgccatgaggcagagaggaacatagctttgcgttcaagtttagaatggaattcgtgtgttgtacagacccttaaggaatatctggagtctttacagtgtttggagtctgataaaagtgactggcgagcaaccaatacagacttactgctggcctatgctaatcttatgcttcttacgctttccactagagatgtgcaggaaagtagagaattactgcaaagttttgatagtgctcttcagtctgtgaaatctttgggtggaaatgatgaactgtcagctactttcttagaaatgaaaggacatttctacatgcatgctggttctctgcttttgaagatgggtcagcatagtagtaatgttcaatggcgagctctttctgagctggctgcattgtgctatctcatagcatttcaggttccaagaccaaagattaaattaataaaaggtgaagctggacaaaatctgctggaaatgatggcctgtgaccgactgagccaatcagggcacatgttgctaaacttaagtcgtggcaagcaagattttttaaaagagattgttgaaacttttgccaacaaaagcgggcagtctgcattatatgatgctctgttttctagtcagtcacctaaggatacatcttttcttggtagcgatgatattggaaacattgatgtacgagaaccagagcttgaagatttgactagatacgatgttggtgctattcgagcacataatggtagtcttcagcaccttacttggcttggcttacagtggaattcattgcctgctttacctggaatccgaaaatggctaaaacagcttttccatcatttgccccatgaaacctcaaggcttgaaacaaatgcacctgaatcaatatgtattttagatcttgaagtatttctccttggagtagtatataccagccacttacaattaaaggagaaatgtaattctcaccacagctcctatcagccgttatgcctgccccttcctgtgtgtaaacagctttgtacagaaagacaaaaatcttggtgggatgcggtttgtactctgattcacagaaaagcagtacctggaaacgtagcaaaattgagacttctagttcagcatgaaataaacactctaagagcccaggaaaaacatggccttcaacctgctctgcttgtacattgggcagaatgccttcagaaaacgggcagcggtcttaattctttttatgatcaacgagaatacatagggagaagtgttcattattggaagaaagttttgccattgttgaagataataaaaaagaagaacagtattcctgaacctattgatcctctgtttaaacattttcatagtgtagacattcaggcatcagaaattgttgaatatgaagaagacgcacacataacttttgctatattggatgcagtaaatggaaatatagaagatgctgtgactgcttttgaatctataaaaagtgttgtttcttattggaatcttgcactgatttttcacaggaaggcagaagacattgaaaatgatgccctttctcctgaagaacaagaagaatgcaaaaattatctgagaaagaccagggactacctaataaagattatagatgacagtgattcaaatctttcagtggtcaagaaattgcctgtgcccctggagtctgtaaaagagatgcttaattcagtcatgcaggaactcgaagactatagtgaaggaggtcctctctataaaaatggttctttgcgaaatgcagattcagaaataaaacattctacaccgtctcctaccaaatattcactatcaccaagtaaaagttacaagtattctcccaaaacaccacctcgatgggcagaagatcagaattctttactgaaaatgatttgccaacaagtagaggccattaagaaagaaatgcaggagttgaaactaaatagcagtaactcagcatcccctcatcgttggcccacagagaattatggaccagactcggtgcctgatggatatcaggggtcacagacatttcatggggctccactaacagttgcaactactggcccttcagtatattatagtcagtcaccagcatataattcccagtatcttctcagaccagcagctaatgttactcccacaaagggcccagtctatggcatgaataggcttccaccccaacagcatatttatgcctatccgcaacagatgcacacaccgccagtgcaaagctcatctgcttgtatgttctctcaggagatgtatggtcctcctgcattgcgttttgagtctcctgcaacgggaattctatcgcccaggggtgatgattactttaattacaatgttcaacagacaagcacaaatccacctttgccagaaccaggatatttcacaaaacctccgattgcagctcatgcttcaagatctgcagaatctaagactatagaatttgggaaaactaattttgttcagcccatgccgggtgaaggattaaggccatctttgccaacacaagcacacacaacacagccaactccttttaaatttaactcaaatttcaaatcaaatgatggtgacttcacgttttcctcaccacaggttgtgacacagccccctcctgcagcttacagtaacagtgaaagccttttaggtctcctgacttcagataaacccttgcaaggagatggctatagtggagccaaaccaattcctggtggtcaaaccattgggcctcgaaatacattcaattttggaagcaaaaatgtgtctggaatttcatttacagaaaacatggggtcgagtcagcaaaagaattctggttttcggcgaagtgatgatatgtttactttccatggtccagggaaatcagtatttggaacacccactttagagacagcaaacaagaatcatgagacagatggaggaagtgcccatggggatgatgatgatgacggtcctcactttgagcctgtagtacctcttcctgataagattgaagtaaaaactggtgaggaagatgaagaagaattcttttgcaaccgcgcgaaattgtttcgtttcgatgtagaatccaaagaatggaaagaacgtgggattggcaatgtaaaaatactgaggcataaaacatctggtaaaattcgccttctaatgagacgagagcaagtattgaaaatctgtgcaaatcattacatcagtccagatatgaaattgacaccaaatgctggatcagacagatcttttgtatggcatgcccttgattatgcagatgagttgccaaaaccagaacaacttgctattaggttcaaaactcctgaggaagcagcactttttaaatgcaagtttgaagaagcccagagcattttaaaagccccaggaacaaatgtagccatggcgtcaaatcaggctgtcagaattgtaaaagaacccacaagtcatgataacaaggatatttgcaaatctgatgctggaaacctgaattttgaatttcaggttgcaaagaaagaagggtcttggtggcattgtaacagctgctcattaaagaatgcttcaactgctaagaaatgtgtatcatgccaaaatctaaacccaagcaataaagagctcgttggcccaccattagctgaaactgtttttactcctaaaaccagcccagagaatgttcaagatcgatttgcattggtgactccaaagaaagaaggtcactgggattgtagtatttgtttagtaagaaatgaacctactgtatctaggtgcattgcgtgtcagaatacaaaatctgctaacaaaagtggatcttcatttgttcatcaagcttcatttaaatttggccagggagatcttcctaaacctattaacagtgatttcagatctgttttttctacaaaggaaggacagtgggattgcagtgcatgtttggtacaaaatgaggggagctctacaaaatgtgctgcttgtcagaatccgagaaaacagagtctacctgctacttctattccaacacctgcctcttttaagtttggtacttcagagacaagtaaaactctaaaaagtggatttgaagacatgtttgctaagaaggaaggacagtgggattgcagttcatgcttagtgcgaaatgaagcaaatgctacaagatgtgttgcttgtcagaatccggataaaccaagtccatctacttctgttccagctcctgcctcttttaagtttggtacttcagagacaagcaaggctccaaagagcggatttgagggaatgttcactaagaaggagggacagtgggattgcagtgtgtgcttagtaagaaatgaagccagtgctaccaaatgtattgcttgtcagaatccaggtaaacaaaatcaaactacttctgcagtttcaacacctgcctcttcagagacaagcaaggctccaaagagcggatttgagggaatgttcactaagaaggagggacagtgggattgcagtgtgtgcttagtaagaaatgaagccagtgctaccaaatgtattgcttgtcagaatccaggtaaacaaaatcaaactacttctgcagtttcaacacctgcctcttcagagacaagcaaggctccaaagagcggatttgagggaatgttcactaagaaggaaggacagtgggattgcagtgtgtgcttagtaagaaatgaagccagtgctaccaaatgtattgcttgtcagtgtccaagtaaacaaaatcaaacaactgcaatttcaacacctgcctcttcggagataagcaaggctccaaagagtggatttgaaggaatgttcatcaggaaaggacagtgggattgtagtgtttgctgtgtacaaaatgagagttcttccttaaaatgtgtggcttgtgatgcctctaaaccaactcataaacctattgcagaagctccttcagctttcacactgggctcagaaatgaagttgcatgactcttctggaagtcaggtgggaacaggatttaaaagtaatttctcagaaaaagcttctaagtttggcaatacagagcaaggattcaaatttgggcatgtggatcaagaaaattcaccttcatttatgtttcagggttcttctaatacagaatttaagtcaaccaaagaaggattttccatccctgtgtctgctgatggatttaaatttggcatttcggaaccaggaaatcaagaaaagaaaagtgaaaagcctcttgaaaatggtactggcttccaggctcaggatattagtggccagaagaatggccgtggtgtgatttttggccaaacaagtagcacttttacatttgcagatcttgcaaaatcaacttcaggagaaggatttcagtttggcaaaaaagaccccaatttcaagggattttcaggtgctggagaaaaattattctcatcacaatacggtaaaatggccaataaagcaaacacttccggtgactttgagaaagatgatgatgcctataagactgaggacagcgatgacatccattttgaaccagtagttcaaatgcccgaaaaagtagaacttgtaacaggagaagaagatgaaaaagttctgtattcacagcgggtaaaactatttagatttgatgctgaggtaagtcagtggaaagaaaggggcttggggaacttaaaaattctcaaaaacgaggtcaatggcaaactaagaatgctgatgcgaagagaacaagtactaaaagtgtgtgctaatcattggataacgactacgatgaacctgaagcctctctctggatcagatagagcatggatgtggttagccagtgatttctctgatggtgatgccaaactagagcagttggcagcaaaatttaaaacaccagagctggctgaagaattcaagcagaaatttgaggaatgccagcggcttctgttagacataccacttcaaactccccataaacttgtagatactggcagagctgccaagttaatacagagagctgaagaaatgaagagtggactgaaagatttcaaaacatttttgacaaatgatcaaacaaaagtcactgaggaagaaaataagggttcaggtacaggtgcggccggtgcctcagacacaacaataaaacccaatcctgaaaacactgggcccacattagaatgggataactatgatttaagggaagatgctttggatgatagtgtcagtagtagctcagtacatgcttctccattggcaagtagccctgtgagaaaaaatcttttccgttttggtgagtcaacaacaggatttaacttcagttttaaatctgctttgagtccatctaagtctcctgccaagttgaatcagagtgggacttcagttggcactgatgaagaatctgatgttactcaagaagaagagagagatggacagtactttgaacctgttgttcctttacctgatctagttgaagtatccagtggtgaggaaaatgaacaagttgtttttagtcacagggcaaaactctacagatatgataaagatgttggtcaatggaaagaaaggggcattggtgatataaagattttacagaattatgataataagcaagttcgtatagtgatgagaagggaccaagtattaaaactttgtgccaatcacagaataactccagacatgactttgcaaaatatgaaagggacagaaagagtatggttgtggactgcatgtgattttgcagatggagaaagaaaagtagagcatttagctgttcgttttaaactacaggatgttgcagactcgtttaagaaaatttttgatgaagcaaaaacagcccaggaaaaagattctttgataacacctcatgtttctcggtcaagcactcccagagagtcaccatgtggcaaaattgctgtagctgtattagaagaaaccacaagagagaggacagatgttattcagggtgatgatgtagcagatgcaacttcagaagttgaagtgtctagcacatctgaaacaacaccaaaagcagtggtttctcctccaaagtttgtatttggttcagagtctgttaaaagcatttttagtagtgaaaaatcaaaaccatttgcattcggcaacagttcagccactgggtctttgtttggatttagttttaatgcacctttgaaaagtaacaatagtgaaactagttcagtagcccagagtggatctgaaagcaaagtggaacctaaaaaatgtgaactgtcaaagaactctgatatcgaacagtcttcagatagcaaagtcaaaaatctctttgcttcctttccaacggaagaatcttcaatcaactacacatttaaaacaccagaaaaggcaaaagagaagaaaaaacctgaagattctccctcagatgatgatgttctcattgtatatgaactaactccaaccgctgagcagaaagcccttgcaaccaaacttaaacttcctccaactttcttctgctacaagaatagaccagattatgttagtgaagaagaggaggatgatgaagatttcgaaacagctgtcaagaaacttaatggaaaactatatttggatggctcagaaaaatgtagacccttggaagaaaatacagcagataatgagaaagaatgtattattgtttgggaaaagaaaccaacagttgaagagaaggcaaaagcagatacgttaaaacttccacctacatttttttgtggagtctgtagtgatactgatgaagacaatggaaatggggaagactttcaatcagagcttcaaaaagttcaggaagctcaaaaatctcagacagaagaaataactagcacaactgacagtgtatatacaggtgggactgaagtgatggtaccttctttctgtaaatctgaagaacctgattctattaccaaatccattagttcaccatctgtttcctctgaaactatggacaaacctgtagatttgtcaactagaaaggaaattgatacagattctacaagccaaggggaaagcaagatagtttcatttggatttggaagtagcacagggctctcatttgcagacttggcttccagtaattctggagattttgcttttggttctaaagataaaaatttccaatgggcaaatactggagcagctgtgtttggaacacagtcagtcggaacccagtcagccggtaaagttggtgaagatgaagatggtagtgatgaagaagtagttcataatgaagatatccattttgaaccaatagtgtcactaccagaggtagaagtaaaatctggagaagaagatgaagaaattttgtttaaagagagagccaaactttatagatgggatcgggatgtcagtcagtggaaggagcgcggtgttggagatataaagattctttggcatacaatgaagaattattaccggatcctaatgagaagagaccaggtttttaaagtgtgtgcaaaccacgttattactaaaacaatggaattaaagcccttaaatgtttcaaataatgctttagtttggactgcctcagattatgctgatggagaagcaaaagtagaacagcttgcagtgagatttaaaactaaagaagtagctgattgtttcaagaaaacatttgaagaatgtcagcagaatttaatgaaactccagaaaggacatgtatcactggcagcagaattatcaaaggagaccaatcctgtggtgttttttgatgtttgtgcggacggtgaacctctagggcggataactatggaattattttcaaacattgttcctcggactgctgagaacttcagagcactatgcactggagagaaaggctttggtttcaagaattccatttttcacagagtaattccagattttgtttgccaaggaggagatatcaccaaacatgatggaacaggcggacagtccatttatggagacaaatttgaagatgaaaattttgatgtgaaacatactggtcctggtttactatccatggccaatcaaggccagaataccaataattctcaatttgttataacactgaagaaagcagaacatttggactttaagcatgtagtatttgggtttgttaaggatggcatggatactgtgaaaaagattgaatcatttggttctcccaaagggtctgtttgtcgaagaataactatcacagaatgtggacagatataaaatcattgttgttcatagaaaatttcatctgtataagcagttggattgaagcttagctattacaatttgatagttatgttcagcttttgaaaatggacgtttccgatttacaaatgtaaaattgcagcttatagctgttgtcactttttaatgtgttataattgaccttgcatggtgtgaaataaaagtttaaacactggtgatttcaggtgtacttgtgtttatgtactcctgacgtattaaaatggaataatactaatcttgttaaaagcaatagacctcaaactattgaaggaatatgatatatgcaatttaattttaattccttttaagatatttggacttcctgcatggatatacttaccatttgaataaagggaccacaacttggataatttaattttaggtttgaaatatatttggtaatcttaactattggtgtactcatttatgcatagagactcgtttatgaatgggtagagccacagaacgtatagagttaaccaaagtgctcttctctagaatctttacacctcctgtgtggttacaagttaactttgtaagtagcgtaccttccttccttaaaatatctagcttcctgtgccctttcatagatattcgattaatttttacattttaaacaagttgactatttcctttaggggttttgtttcaaacttttctgtcatctgtctctactacctcagaaactgcagcttggttctgatgatagaaattgaatttttccttgtagttattgtgataaagtatgaatatttttagaaagtctataccatgttctttcgttaaagatttgctttatacaagattgttgcagtacctttttctggtaaattttgtagcagaaataaaatgacaattcctaagagccaaaa

SEQ ID NO: 8 - protein, 3224 aa, Artificial Sequence
  Description of Artificial Sequence/note = synthetic construct
MRRSKADVERYIASVQGSTPSPRQKSMKGFYFAKLYYEAKEYDLAKKYICTYINVQERDPKAHRFLGLLYELEENTDKAVECYRRSVELNPTQKDLVLKIAELLCKNDVTDGRAKYWLERAAKLFPGSPAIYKLKEQLLDCEGEDGWNKLFDLIQSELYVRPDDVHVNIRLVEVYRSTKRLKDAVAHCHEAERNIALRSSLEWNSCVVQTLKEYLESLQCLESDKSDWRATNTDLLLAYANLMLLTLSTRDVQESRELLQSFDSALQSVKSLGGNDELSATFLEMKGHFYMHAGSLLLKMGQHSSNVQWRALSELAALCYLIAFQVPRPKIKLIKGEAGQNLLEMMACDRLSQSGHMLLNLSRGKQDFLKEIVETFANKSGQSALYDALFSSQSPKDTSFLGSDDIGNIDVREPELEDLTRYDVGAIRAHNGSLQHLTWLGLQWNSLPALPGIRKWLKQLFHHLPHETSRLETNAPESICILDLEVFLLGVVYTSHLQLKEKCNSHHSSYQPLCLPLPVCKQLCTERQKSWWDAVCTLIHRKAVPGNVAKLRLLVQHEINTLRAQEKHGLQPALLVHWAECLQKTGSGLNSFYDQREYIGRSVHYWKKVLPLLKIIKKKNSIPEPIDPLFKHFHSVDIQASEIVEYEEDAHITFAILDAVNGNIEDAVTAFESIKSVVSYWNLALIFHRKAEDIENDALSPEEQEECKNYLRKTRDYLIKIIDDSDSNLSVVKKLPVPLESVKEMLNSVMQELEDYSEGGPLYKNGSLRNADSEIKHSTPSPTKYSLSPSKSYKYSPKTPPRWAEDQNSLLKMICQQVEAIKKEMQELKLNSSNSASPHRWPTENYGPDSVPDGYQGSQTFHGAPLTVATTGPSVYYSQSPAYNSQYLLRPAANVTPTKGPVYGMNRLPPQQHIYAYPQQMHTPPVQSSSACMFSQEMYGPPALRFESPATGILSPRGDDYFNYNVQQTSTNPPLPEPGYFTKPPIAAHASRSAESKTIEFGKTNFVQPMPGEGLRPSLPTQAHTTQPTPFKFNSNFKSNDGDFTFSSPQVVTQPPPAAYSNSESLLGLLTSDKPLQGDGYSGAKPIPGGQTIGPRNTFNFGSKNVSGISFTENMGSSQQKNSGFRRSDDMFTFHGPGKSVFGTPTLETANKNHETDGGSAHGDDDDDGPHFEPVVPLPDKIEVKTGEEDEEEFFCNRAKLFRFDVESKEWKERGIGNVKILRHKTSGKIRLLMRREQVLKICANHYISPDMKLTPNAGSDRSFVWHALDYADELPKPEQLAIRFKTPEEAALFKCKFEEAQSILKAPGTNVAMASNQAVRIVKEPTSHDNKDICKSDAGNLNFEFQVAKKEGSWWHCNSCSLKNASTAKKCVSCQNLNPSNKELVGPPLAETVFTPKTSPENVQDRFALVTPKKEGHWDCSICLVRNEPTVSRCIACQNTKSANKSGSSFVHQASFKFGQGDLPKPINSDFRSVFSTKEGQWDCSACLVQNEGSSTKCAACQNPRKQSLPATSIPTPASFKFGTSETSKTLKSGFEDMFAKKEGQWDCSSCLVRNEANATRCVACQNPDKPSPSTSVPAPASFKFGTSETSKAPKSGFEGMFTKKEGQWDCSVCLVRNEASATKCIACQNPGKQNQTTSAVSTPASSETSKAPKSGFEGMFTKKEGQWDCSVCLVRNEASATKCIACQNPGKQNQTTSAVSTPASSETSKAPKSGFEGMFTKKEGQWDCSVCLVRNEASATKCIACQCPSKQNQTTAISTPASSEISKAPKSGFEGMFIRKGQWDCSVCCVQNESSSLKCVACDASKPTHKPIAEAPSAFTLGSEMKLHDSSGSQVGTGFKSNFSEKASKFGNTEQGFKFGHVDQENSPSFMFQGSSNTEFKSTKEGFSIPVSADGFKFGISEPGNQEKKSEKPLENGTGFQAQDISGQKNGRGVIFGQTSSTFTFADLAKSTSGEGFQFGKKDPNFKGFSGAGEKLFSSQYGKMANKANTSGDFEKDDDAYKTEDSDDIHFEPVVQMPEKVELVTGEEDEKVLYSQRVKLFRFDAEVSQWKERGLGNLKILKNEVNGKLRMLMRREQVLKVCANHWITTTMNLKPLSGSDRAWMWLASDFSDGDAKLEQLAAKFKTPELAEEFKQKFEECQRLLLDIPLQTPHKLVDTGRAAKLIQRAEEMKSGLKDFKTFLTNDQTKVTEEENKGSGTGAAGASDTTIKPNPENTGPTLEWDNYDLREDALDDSVSSSSVHASPLASSPVRKNLFRFGESTTGFNFSFKSALSPSKSPAKLNQSGTSVGTDEESDVTQEEERDGQYFEPVVPLPDLVEVSSGEENEQVVFSHRAKLYRYDKDVGQWKERGIGDIKILQNYDNKQVRIVMRRDQVLKLCANHRITPDMTLQNMKGTERVWLWTACDFADGERKVEHLAVRFKLQDVADSFKKIFDEAKTAQEKDSLITPHVSRSSTPRESPCGKIAVAVLEETTRERTDVIQGDDVADATSEVEVSSTSETTPKAVVSPPKFVFGSESVKSIFSSEKSKPFAFGNSSATGSLFGFSFNAPLKSNNSETSSVAQSGSESKVEPKKCELSKNSDIEQSSDSKVKNLFASFPTEESSINYTFKTPEKAKEKKKPEDSPSDDDVLIVYELTPTAEQKALATKLKLPPTFFCYKNRPDYVSEEEEDDEDFETAVKKLNGKLYLDGSEKCRPLEENTADNEKECIIVWEKKPTVEEKAKADTLKLPPTFFCGVCSDTDEDNGNGEDFQSELQKVQEAQKSQTEEITSTTDSVYTGGTEVMVPSFCKSEEPDSITKSISSPSVSSETMDKPVDLSTRKEIDTDSTSQGESKIVSFGFGSSTGLSFADLASSNSGDFAFGSKDKNFQWANTGAAVFGTQSVGTQSAGKVGEDEDGSDEEVVHNEDIHFEPIVSLPEVEVKSGEEDEEILFKERAKLYRWDRDVSQWKERGVGDIKILWHTMKNYYRILMRRDQVFKVCANHVITKTMELKPLNVSNNALVWTASDYADGEAKVEQLAVRFKTKEVADCFKKTFEECQQNLMKLQKGHVSLAAELSKETNPVVFFDVCADGEPLGRITMELFSNIVPRTAENFRALCTGEKGFGFKNSIFHRVIPDFVCQGGDITKHDGTGGQSIYGDKFEDENFDVKHTGPGLLSMANQGQNTNNSQFVITLKKAEHLDFKHVVFGFVKDGMDTVKKIESFGSPKGSVCRRITITECGQI

SEQ ID NO: 9 - DNA, 4346 bp, Artificial Sequence
  Description of Artificial Sequence/note = synthetic construct
aagctggagcggcggtggcggcggcgtagggaggccggggccggggctgaggccgcggccgggtctgcgaggcccttggggcggcaggcggcggcggcccggggctcgagcccggaggcaggagcagccgccatggccgagagcatcataattcgtgtccagtccccagatggagtgaagcggatcacagcaacaaagagagaaacagcagcaacatttttgaaaaaggttgcaaaggagtttggcttccaaaataatggcttctcggtttacatcaatagaaacaagactggagagataacagcctcctccaacaaatccctcaacttgctaaaaatcaagcatggcgatttgttgttcctgtttccctcgagccttgctgggccctcatctgaaatggagacgtcagttccaccgggcttcaaagtctttggcgctcccaacgtggtggaggatgagattgatcagtacctcagcaaacaggacgggaagatttacagaagccgagacccacagctatgccgccacggccctttggggaaatgcgtgcactgcgtccctctagagccattcgatgaggactatctaaaccatctcgagcctcccgtgaagcacatgtccttccacgcctacatccggaagctgactggaggggctgacaaggggaagtttgttgccctggagaacatcagctgcaagattaagtcagggtgcgaggggcacctcccgtggccgaatggcatctgtactaagtgccagccgagcgccatcacgctgaacagacagaagtacaggcatgtggacaatatcatgtttgagaatcacaccgtcgctgaccgctttcttgacttctggagaaagacagggaaccagcaatttgggtacttatacggacggtacaccgagcacaaagacattccccttggcatcagggctgaagtggctgcgatttatgagccacctcagattggtacacagaacagcttggagcttcttgaggatccaaaagctgaagtggtcgatgaaattgctgccaaacttggcctgcggaaggttggctggatatttacagacctcgtctcagaagatacccgaaagggtaccgtccgctacagtcgaaataaggacacctatttcctaagttcagaagagtgcatcactgcaggagacttccagaacaagcatcccaacatgtgccggctctctccagacggacattttggatccaagtttgttactgcagtggctacaggtggtcctgacaaccaagtccactttgaagggtaccaggtgtccaatcagtgtatggcactggtccgtgatgagtgtttgctgccatgcaaggacgccccggagcttggctacgccaaggagtctagcagtgagcagtacgtgcctgatgtgttttataaggacgtagacaagtttggcaacgagatcacccagctggcccggcccctgcctgtggagtatctcatcatagacatcacaacaactttccccaaggatccagtttacactttttctatttcgcaaaatccatttcctattgaaaaccgggatgtattgggtgagacacaggacttccatagcttggccacctatttgtctcagaatacctcatctgtgttcttggataccatctcagatttccacctcttgctgttcctggtcaccaatgaagttatgcctctgcaggacagcatcagcttgctgctggaggccgtgcggaccagaaatgaggagctcgcccagacatggaagaggtctgagcagtgggccaccatcgagcagctgtgcagtacagttggcgggcagctcccaggtctccatgagtacggggccgtcgagggctccacacacacggccactgcagccatgtgggcctgtcagcactgcacgttcatgaaccagccaggcacaggccactgcgagatgtgcagcctccccaggacctagggcgcctgccctctgctggctaggaccgggcccagcccagcccttcctgaagccagaagcgttgctgagtgtgttccctgtaactgccccatagtgggcagccctggaggaacaaggggctggctgtcctgggctccctgacccactgaagcttctcagcacgttcctccctggagagcgggcgccacggctggtattctgcaggctgaatgcagtctccagactggaaacgcagagcggctcctcacgcctaatcctgttgacaagtcccccgccgtgttggaaagacctctcgcctctacgtggcacctggaattggggcgcacagggaggggcggatgctgccaccacccagccttctctttattttcagtgctttttgtttggcctccctgcccccatgtttttctggctgggagctcttggttacccccttgtctgactgcttggtggcaggcaccctgcctgtgagggccactttcctcttctagtagctgttgtctggtggggacgcagtggccctggacacggctcccctctgcaggtctgcaggtcggtttgctgcctgccctcctcctctcacccgatgtccaggtgggattttaaagtctgcattggttataacaacagttatcagtaattcctgcccagaagacttttatttatttttttttaagataaaaactgcacaaaaggggagtgagagagactagtttccacatccttccctcctttagtgaagcccccgaggttgtgtccagggtgatgagtgtggacgggggcaccagtcagttctcccttgaagtaaacctcagtgcctgagacttttctaccaagccacacagctgcagcaactgcagatactgcgggctgagcaggagcagtggtggcgcctgccctgaggctgccctgcgatggcctgggtgggagagctgggtcaccggtgccgatgctctggccctccccaatacgctgcttcgtcccactgcaccgcctggctgagggcgttaggggctgtgcctcttgtgagggccatttgggacctccctggggcactgcacaattgatagtgtaccaatagggggagactgggcgatctggaacagcacgtggtggggtcctgcttgtgtgctctgcgttcctctgtggcgtggccaggccggggcctggctcttacccggggagtggtgggcatctcgatgcttctttgccttaatgatggccacatctgggctgctctgcacccacgggagaggctggcccagctgcagactgcttagggacttctgtgtccatcctggggggtaagcccacgtgacccacattcttggcactatgaacagagaacatttgcctgttggcttctgaagtggtcagggccatggctgacacctccaggtccgcctggcatgggacaccaagtggaaggcccaagcagctcatctgctcttgggaccaggggccagttgggttgggtctggtcacggcagagcttttgtggagggtcaggaagggtggagaggagctgggttgaagcggactgctgcggatgcaactcccagcttgcccaccgcgggctgtctgctctccctcctagcagctgtcacactgaagttttgtcctctgctgtctcctctggtcctgagatgagctgtgagcctaggtggccaaggcttcctgcattgcttccctgtgagtccaaggccttcccccaccactgggcagaggctggacagcacggacttctagagagagccgcgttgccagttcctctcccactcgctcgtccttatccaccacgctattatagtttccgttgtcctccaccagcatttcccttactctgaagttccggcattcacatcattcatgttttcttttgtcttttagctaaaggaaaagcattggcgatttgtctgattctggttttgagttactctttgttcagtaatgcactttattttattgtccaaagagagtcagagctaagcatacaggcttgggggtgagccctgctgtgagagttcaggccctgggaggctcagccacctcctcttgtgggaaggaggtctcagccccacctcgcatcttcacctgcccttggtgtggacacaccctctcatgctaccagcaccataatccagtgggggtgactgggtgcacacctgcccaggtgaacacagcggctgccagtctcctggtcccgagaggaggtggggcctggccctggctccctccaaccagctgctcctgggacacaggtgctcctgcttcggctctgtttcggctcacaggtgtgcatcactgggcttggatttgcattacattgaccccagccctgcagtggaacctaataaaagcgcctgaagcaaaaaaaaaaaaaaaaa

SEQ ID NO: 10 - protein, 608 aa, Artificial Sequence
  Description of Artificial Sequence/note = synthetic construct
MAESIIIRVQSPDGVKRITATKRETAATFLKKVAKEFGFQNNGFSVYINRNKTGEITASSNKSLNLLKIKHGDLLFLFPSSLAGPSSEMETSVPPGFKVFGAPNVVEDEIDQYLSKQDGKIYRSRDPQLCRHGPLGKCVHCVPLEPFDEDYLNHLEPPVKHMSFHAYIRKLTGGADKGKFVALENISCKIKSGCEGHLPWPNGICTKCQPSAITLNRQKYRHVDNIMFENHTVADRFLDFWRKTGNQQFGYLYGRYTEHKDIPLGIRAEVAAIYEPPQIGTQNSLELLEDPKAEVVDEIAAKLGLRKVGWIFTDLVSEDTRKGTVRYSRNKDTYFLSSEECITAGDFQNKHPNMCRLSPDGHFGSKFVTAVATGGPDNQVHFEGYQVSNQCMALVRDECLLPCKDAPELGYAKESSSEQYVPDVFYKDVDKFGNEITQLARPLPVEYLIIDITTTFPKDPVYTFSISQNPFPIENRDVLGETQDFHSLATYLSQNTSSVFLDTISDFHLLLFLVTNEVMPLQDSISLLLEAVRTRNEELAQTWKRSEQWATIEQLCSTVGGQLPGLHEYGAVEGSTHTATAAMWACQHCTFMNQPGTGHCEMCSLPRT

SEQ ID NO: 11 - DNA, 3067 bp, Artificial Sequence
  Description of Artificial Sequence/note = synthetic construct
ggtgggtttatctcaaggcctgagtagccggtaacaaacgagggttcccgggattggaccgacgcaccatgcctctgcgacttgatatcaaaagaaagctaactgctagatctgatcgagttaagagtgtggatctgcatcctacagagccatggatgttggcaagtctttacaatggcagtgtgtgtgtttggaatcatgaaacacagacactggtgaagacatttgaagtatgtgatcttcctgttcgagctgcaaagtttgttgcaaggaagaattgggttgtgacaggagcggatgacatgcagattagagtgttcaattacaatactctggagagagttcatatgtttgaagcacactcagactacattcgctgtattgctgttcatccaacccagcctttcattctaactagcagtgatgacatgcttattaagctctgggactgggataaaaaatggtcttgctcacaagtgtttgaaggacacacccattatgttatgcagattgtgatcaaccccaaagataacaatcagtttgccagtgcctctttggacaggactatcaaggtgtggcagttgggctcttcgtcaccaaacttcactttggaaggacatgagaaaggcgtgaattgcattgattactacagtggtggggacaagccatacctcatttcaggtgcagatgaccgtcttgttaaaatatgggattatcagaataaaacatgtgtgcagacactggaaggacatgcccaaaatgtgtcttgtgccagctttcatcctgagttgccaatcattatcacaggttcagaagatggaacagtacgtatttggcattcaagcacctaccggcttgagagcacactgaattatggaatggagagggtatggtgcgtggccagtctaagagggtcaaacaatgtcgctttgggctatgatgaagggagcatcattgttaagcttggtcgggaggaacctgccatgtccatggatgccaatggaaagataatttgggccaagcattcagaagtccagcaggccaacctaaaagcaatgggagatgctgaaattaaagatggtgaaagattgccactggcagtaaaggatatgggcagttgtgaaatataccctcagactattcagcacaatcctaatgggcggtttgtggtggtgtgtggtgatggggagtatatcatctacacagcaatggcattgagaaacaagagctttggatctgctcaggagtttgcatgggcccacgattcttcagagtatgcaataagagagagcaacagcattgtaaagatatttaagaactttaaggaaaaaaaatcatttaaaccagattttggagcagaaagtatctacggcggcttcttattgggagtcagatctgtaaatggcttagccttctatgactgggacaatacagaactcatacgaagaattgaaattcagcccaaacatattttctggtctgactctggagagctagtctgtattgctactgaggaatcattttttatccttaagtatctgtcagaaaaagtcttggctgcacaggaaacacatgagggagttactgaagatggcattgaagatgcctttgaggttcttggtgagattcaggaaattgtgaaaacagggctttgggtaggcgattgcttcatttacacaagttctgtgaacagattaaattattatgttggaggagaaatagtcaccattgcccacttggacaggacgatgtatctcctaggctacattcctaaagacaacaggctttatctgggggataaagaattgaacatcattagctattccctgctggtttcagtcctggaataccagacagctgtcatgcggagggactttagcatggctgataaggtccttcctaccattccaaaagaacagaggaccagagttgcacactttttggaaaagcagggcttcaagcagcaagctcttacagtatccacagatcctgagcatcgttttgagcttgctcttcagcttggagagttaaaaattgcataccagttagcagtggaagcagagtcagaacagaagtggaaacaacttgctgaacttgccattagtaaatgtcagtttggcctagcccaggagtgcctgcatcatgcacaggattatgggggcctgctgcttttggccactgcctctggaaatgctaatatggtgaacaagctagcagagggtgcggagagagatggcaaaaataatgtggcattcatgagctactttttacagggcaaggttgatgcctgcctagagctcttaattagaactggacggctgccagaagctgccttcttggcccgaacttacttacccagtcaggtttcaagggtagtgaaactctggagagagaatctctcaaaagtcaatcagaaagcagcagaatcccttgctgacccaacagagtatgaaaacctgttccctggattaaaagaagcctttgttgttgaagaatgggtgaaggaaacacatgctgatctgtggccagccaaacaatacccacttgtcacgccaaatgaagagagaaatgtcatggaagagggaaaagactttcagccctcaagatctacagctcaacaggaacttgatgggaaacctgcttctcctactccggttattgtggcctcccacacagccaacaaagaagaaaagagtttactcgaactagaagtagatttggataatttggaattagaagatattgacacaacagatatcaatctggatgaagatattttggatgattgactgtaatgctttccatttacctgactaaacagatcattattatatataggtattgattgctaccctgaccacagtgctttggactatgagaaacttcttagatttttatatgtaaatgctgtggaccactgggagcacaatgcccacatcatcttaagaagagtttatgtgcagcatttaaatcactgtgttttccttgttaactaaaacagacatgggctttgatttttttcatactattagaccatatctcataaaaccttttgaattaaaaaaaa

SEQ ID NO: 12 - protein, 906 aa, Artificial Sequence
  Description of Artificial Sequence/note = synthetic construct
MPLRLDIKRKLTARSDRVKSVDLHPTEPWMLASLYNGSVCVWNHETQTLVKTFEVCDLPVRAAKFVARKNWVVTGADDMQIRVFNYNTLERVHMFEAHSDYIRCIAVHPTQPFILTSSDDMLIKLWDWDKKWSCSQVFEGHTHYVMQIVINPKDNNQFASASLDRTIKVWQLGSSSPNFTLEGHEKGVNCIDYYSGGDKPYLISGADDRLVKIWDYQNKTCVQTLEGHAQNVSCASFHPELPIIITGSEDGTVRIWHSSTYRLESTLNYGMERVWCVASLRGSNNVALGYDEGSIIVKLGREEPAMSMDANGKIIWAKHSEVQQANLKAMGDAEIKDGERLPLAVKDMGSCEIYPQTIQHNPNGRFVVVCGDGEYIIYTAMALRNKSFGSAQEFAWAHDSSEYAIRESNSIVKIFKNFKEKKSFKPDFGAESIYGGFLLGVRSVNGLAFYDWDNTELIRRIEIQPKHIFWSDSGELVCIATEESFFILKYLSEKVLAAQETHEGVTEDGIEDAFEVLGEIQEIVKTGLWVGDCFIYTSSVNRLNYYVGGEIVTIAHLDRTMYLLGYIPKDNRLYLGDKELNIISYSLLVSVLEYQTAVMRRDFSMADKVLPTIPKEQRTRVAHFLEKQGFKQQALTVSTDPEHRFELALQLGELKIAYQLAVEAESEQKWKQLAELAISKCQFGLAQECLHHAQDYGGLLLLATASGNANMVNKLAEGAERDGKNNVAFMSYFLQGKVDACLELLIRTGRLPEAAFLARTYLPSQVSRVVKLWRENLSKVNQKAAESLADPTEYENLFPGLKEAFVVEEWVKETHADLWPAKQYPLVTPNEERNVMEEGKDFQPSRSTAQQELDGKPASPTPVIVASHTANKEEKSLLELEVDLDNLELEDIDTTDINLDEDILDD

SEQ ID NO: 13 - DNA, 914 bp, Artificial Sequence
  Description of Artificial Sequence/note = synthetic construct
ggcggcgagcggaatgcagcggcccgaggcctggccacgtccgcacccgggggagggggccgcggcggcccaggccgggggcccggcgccgcctgctcgagccggggagccctcggggctgcggttgcaggaaccttccctctacaccatcaaggctgttttcatcctagataatgacgggcgccggctgctggccaagtattatgatgacacattcccctccatgaaggagcagatggttttcgagaaaaatgtcttcaacaagaccagccggactgagagtgagattgcattttttgggggtatgaccatcgtctacaagaacagcattgacctcttcctatacgtggtgggctcatcctacgagaatgagctgatgctcatgtctgttctcacctgcctgtttgagtctctgaaccacatgttaaggaagaacgtggagaagcgctggttgctggagaacatggacggagccttcttggtgctggacgagattgtggatggcggtgtgattctggagagtgacccccagcaagtgatccagaaggtgaattttagggcagatgatggcggcttgactgaacagagtgtggcccaggttcttcagtctgccaaggaacaaattaaatggtcgttattgaaatgaaggctgtggattcaaggctccctgccccccagaccatttccccaatcctggcaaaagcccaaagatcccagggtcaggagagacccctctgtatccccaggtccctcccagaactgactcctaaggtctccagccagggcttctgagatgcaaaggtttggcctcaggagagtcaccttttctcacggccctggccttaactcatatcttaggcattcctggccccagggccctaataaacctgcttttgtcttctgcaaaaaaaaaa

SEQ ID NO: 14 - protein, 210 aa, Artificial Sequence
  Description of Artificial Sequence/note = synthetic construct
MQRPEAWPRPHPGEGAAAAQAGGPAPPARAGEPSGLRLQEPSLYTIKAVFILDNDGRRLLAKYYDDTFPSMKEQMVFEKNVFNKTSRTESEIAFFGGMTIVYKNSIDLFLYVVGSSYENELMLMSVLTCLFESLNHMLRKNVEKRWLLENMDGAFLVLDEIVDGGVILESDPQQVIQKVNFRADDGGLTEQSVAQVLQSAKEQIKWSLLK

SEQ ID NO: 15 - DNA, 5064 bp, Artificial Sequence
  Description of Artificial Sequence/note = synthetic construct
gagaaggggaccttcaggtccaggcaaagggggaacttctgtcgtgggaacgaaaaagaaagaggatttacagggtggggggacagaggggcagcaggaaccagaagggagacagtggcggtcgcaccggggccgatccgagagttccccttagagaacggagctcacgggcggggaggcctcacctgctagtaggacgcagaaagacagaaggcgaaggagaccccctgccgtagccatcttgcctctctgctgagcggaagcccccgttcggctcctgtctgttagcggcctctctaggctaccactgacaccgtctctgtggcccggagcctaagagaccggaagttcgtgtttccaggcgcttccggaaaccgcgggagagggtcgctgacgtggaggcgtccgaagggcagcagggtgtgtcggggctcggattaagacatcggagtcggagacctgagagatgttaaccaaattcgagaccaagagcgcgcgggtcaaagggctcagctttcaccccaaaagaccttggatcctgactagtttacataatggggtcatccagttatgggactatcggatgtgcactctcattgacaagtttgatgaacatgatggtccagtgcgaggcattgacttccataagcagcagccactgttcgtctctggaggagatgactataagattaaggtttggaattacaagcttcggcgctgtcttttcacattgcttgggcacttagattatattcgcaccacgttttttcatcatgaatatccctggattctgagtgcctccgatgatcagaccatccgagtgtggaattggcaatctagaacctgtgtttgtgtgttaacagggcacaaccattatgtgatgtgtgctcagttccaccccacagaagacttggtagtatcagccagcctggaccagactgtgcgcgtttgggatatttctggtctgaggaaaaaaaacctgtcccctggtgcggtggaatcggatgtgagaggaataactggggttgatctatttggaactacagatgcagtggtgaagcatgtactagagggtcacgatcgtggagtaaactgggctgccttccaccccactatgccccttattgtatctggggcagatgatcgtcaagtgaagatctggcgcatgaatgaatcaaaggcatgggaggttgatacctgccggggccattacaacaatgtatcttgtgccgtcttccaccctcgccaagagttgatcctcagcaattctgaggacaagagtattcgagtctgggatatgtctaagcggactggggttcagactttccgcagagaccatgatcgtttctgggtcctagctgctcaccctaaccttaacctctttgcagcaggccatgatggtggtatgattgtgtttaagctggaacgggaacggccagcctatgctgttcatggcaatatgctacactatgtcaaggaccgattcttacgacagctggatttcaacagctccaaagatgtagctgtgatgcagttgcggagtggttccaagtttccagtattcaatatgtcatacaatccagcagaaaatgcagtcctgctttgtacaagagctagcaatctagagaatagtacctatgacctgtacaccatccctaaagatgctgactcccagaatcctgatgcgcctgaagggaaacgatcctcaggcctgacagccgtttgggtcgctcgaaatcggtttgctgtcctagatcggatgcattcgcttctgatcaagaatctgaagaatgagatcaccaaaaaggtacaggtgcccaactgtgatgagatcttctatgctggcacaggcaatctcctgcttcgagatgcggactctatcacactctttgacgtacagcagaagcggactctggcatctgtgaagatttctaaagtgaaatacgttatctggtcagcagacatgtcacatgtagcactactagccaaacacgccattgtgatctgtaaccgcaaactggatgctttatgtaacattcatgagaacattcgtgtcaagagtggggcctgggatgagagtggggtatttatctataccacaagcaaccacatcaaatatgctgtcaccactggggaccacgggatcattcgaactctggatttacccatctatgtcacacgggtgaagggcaacaatgtatactgcctagacagggagtgtcgtccccgggtactcaccattgatcccactgagttcaaattcaagctggccctgatcaacagaaaatatgatgaggtactgcacatggtgaggaatgccaaactagttggccagtctattattgcttatctccagaagaagggctatcctgaagtggcactgcattttgtcaaggatgagaaaactcgctttagtctggcactggagtgtggaaacattgagattgctctggaagcagccaaagcactggatgacaagaactgctgggaaaagctgggagaagtggccctgctgcaggggaaccaccagattgtggaaatgtgctatcagcgtaccaaaaactttgacaaagtttccttcctgtatcttatcactggcaacttagaaaaacttcgcaagatgatgaagattgctgagatcagaaaggacatgagtggccactatcagaatgccctatacctgggtgatgtgtcagagcgtgtgcggatcctgaagaactgtggacagaagtccctggcctatctcacagctgctacccatggcttagatgaagaagctgagagcctaaaggagacatttgacccagagaaggagacaatcccagacattgaccctaatgccaagctgctccagccacctgcacctatcatgccattggataccaattggcctttattgactgtatccaaaggattttttgaaggcaccattgccagcaaagggaagggaggagcactggctgctgacattgacattgacactgttggtacagagggctggggagaggatgcagagctgcagttggatgaagatgggtttgtggaggctacagaaggtttgggggatgatgctcttggcaagggacaggaagaaggaggtggctgggatgtagaagaagatctggagctccctcctgagctggatatatcccctggggcagctggtggggctgaagatggtttctttgtgcccccaaccaagggaacaagtccaactcagatctggtgtaataactctcagcttccagttgatcacatcctggcaggctctttcgaaacagccatgcggctccttcatgaccaagtaggggtaatccagtttggcccctacaagcaactgttcctacagacatacgcccgaggccgcacaacctatcaggctctgccctgcctaccctccatgtatggctatcctaatcgcaactggaaggatgcagggctgaagaatggtgtaccagctgtgggcctgaagcttaatgacctcatccaacggttgcagctgtgctaccagctcaccacagttggcaaatttgaggaggctgtggaaaaattccgttccatccttctcagtgtgccacttcttgttgtggacaataaacaagagattgcagaggcccagcagctcatcaccatttgccgtgagtacattgtgggtttgtccgtggagacagaaaggaagaagctgcccaaagagactctagaacagcagaagcgcatctgtgagatggcagcctatttcacccactcaaacctgcagcctgtgcacatgatcctggtgctgcgtacagccctcaatctgttcttcaagctcaagaacttcaagacagctgccacctttgctcggcgcctactagaactcgggcccaagcctgaggtggcccaacagacccgaaaaatcctgtctgcctgtgagaagaatcccacagatgcctaccagctcaattatgacatgcacaacccctttgacatttgtgctgcatcatatcggcccatctaccgtggaaagccagtagaaaagtgtccactcagtggggcctgctattcccctgagttcaaaggtcaaatctgcagggtcaccacagtgacagagattggcaaagatgtgattggtttaaggatcagtcctctgcagtttcgctaaggccccctttgtgtgcatgggtcagtcaccatatgttccccccagagaatgtgtctatatcctccttctaacagcaccttccccctgcagctactcttcagatctggctctctgtaccctaaaacctagtatctttttctcttctatggaaaatccgaaggtctaaacttgacttttttgaggtcttctcaacttgactacagttgtgctcataattgtccttgcctttccagcttaattattttaaggaacaaatgaaaactctgggctgggtggagtggctcatacctgtaatcccagcactttgggaggctacggtgggcagatcatctgaggccaggagttcgagacctgcctggccaacatggcaacaccccgtctctaataaaaatataaaaattagcctggcatggtagcatgcgcctatagtcccagctgctcaggaggctgaggcatgagaatcgcttgaacctaggaggtggaggttgcattcaactgagatcataccacttcattccagcctgggtgacagagcaagactctgtctcaaaaaaaaaaaaaaaaaaaaaaaaaaaaaaaaaaggaaaactctgtgatggacatttgtttagtaaatcccttcagtatttatccctcctttccccacagcagctttctttcctgtcaactagaaaggagcaggatgtaataaatacattttggtgtgactaggccacaccaactcttaatcatctcccattttccttagacatttaaatttcaaggcaggtaccctctgtgtactcagaaatttgaagaagttatttggttttccaaaatgcacactgcgggttattgatttgttctttacaactattgttctcatatttctcacactaaataaatctctatgagagcttcttgaaaaaaaaaaaaaaaaagcg

SEQ ID NO: 16 - protein, 1224 aa, Artificial Sequence
  Description of Artificial Sequence/note = synthetic construct
MLTKFETKSARVKGLSFHPKRPWILTSLHNGVIQLWDYRMCTLIDKFDEHDGPVRGIDFHKQQPLFVSGGDDYKIKVWNYKLRRCLFTLLGHLDYIRTTFFHHEYPWILSASDDQTIRVWNWQSRTCVCVLTGHNHYVMCAQFHPTEDLVVSASLDQTVRVWDISGLRKKNLSPGAVESDVRGITGVDLFGTTDAVVKHVLEGHDRGVNWAAFHPTMPLIVSGADDRQVKIWRMNESKAWEVDTCRGHYNNVSCAVFHPRQELILSNSEDKSIRVWDMSKRTGVQTFRRDHDRFWVLAAHPNLNLFAAGHDGGMIVFKLERERPAYAVHGNMLHYVKDRFLRQLDFNSSKDVAVMQLRSGSKFPVFNMSYNPAENAVLLCTRASNLENSTYDLYTIPKDADSQNPDAPEGKRSSGLTAVWVARNRFAVLDRMHSLLIKNLKNEITKKVQVPNCDEIFYAGTGNLLLRDADSITLFDVQQKRTLASVKISKVKYVIWSADMSHVALLAKHAIVICNRKLDALCNIHENIRVKSGAWDESGVFIYTTSNHIKYAVTTGDHGIIRTLDLPIYVTRVKGNNVYCLDRECRPRVLTIDPTEFKFKLALINRKYDEVLHMVRNAKLVGQSIIAYLQKKGYPEVALHFVKDEKTRFSLALECGNIEIALEAAKALDDKNCWEKLGEVALLQGNHQIVEMCYQRTKNFDKVSFLYLITGNLEKLRKMMKIAEIRKDMSGHYQNALYLGDVSERVRILKNCGQKSLAYLTAATHGLDEEAESLKETFDPEKETIPDIDPNAKLLQPPAPIMPLDTNWPLLTVSKGFFEGTIASKGKGGALAADIDIDTVGTEGWGEDAELQLDEDGFVEATEGLGDDALGKGQEEGGGWDVEEDLELPPELDISPGAAGGAEDGFFVPPTKGTSPTQIWCNNSQLPVDHILAGSFETAMRLLHDQVGVIQFGPYKQLFLQTYARGRTTYQALPCLPSMYGYPNRNWKDAGLKNGVPAVGLKLNDLIQRLQLCYQLTTVGKFEEAVEKFRSILLSVPLLVVDNKQEIAEAQQLITICREYIVGLSVETERKKLPKETLEQQKRICEMAAYFTHSNLQPVHMILVLRTALNLFFKLKNFKTAATFARRLLELGPKPEVAQQTRKILSACEKNPTDAYQLNYDMHNPFDICAASYRPIYRGKPVEKCPLSGACYSPEFKGQICRVTTVTEIGKDVIGLRISPLQFR

SEQ ID NO: 17 - DNA, 1208 bp, Artificial Sequence
  Description of Artificial Sequence/note = synthetic construct
ctggtgagaagacagcgaaatggcgcctccggcccccggcccggcctccggcggctccggggaggtagacgagctgttcgacgtaaagaacgccttctacatcggcagctaccagcagtgcataaacgaggcgcagcgggtgaagctgtcaagcccagagagagacgtggagagggacgtcttcctgtatagagcgtacctggcgcagaggaagttcggtgtggtcctggatgagatcaagccctcctcggcccctgagctccaggccgtgcgcatgtttgctgactacctcgcccacgagagtcggagggacagcatcgtggccgagctggaccgagagatgagcaggagcgtggacgtgaccaacaccaccttcctgctcatggccgcctccatctatctccacgaccagaacccggatgccgccctgcgtgcgctgcaccagggggacagcctggagtgcacagccatgacagtgcagatcctgctgaagctggaccgcctggacctcgcccggaaggagctgaagagaatgcaggacctggacgaggatgccaccctcacccagctcgccactgcctgggtcagcctggccacgggtggtgagaagctgcaggatgcctactacatcttccaggagatggctgacaagtgctcgcccaccctgctgctgctcaatgggcaggcggcctgccacatggcccagggccgctgggaggccgctgagggcctgctgcaggaggcgctagacaaggatagtggctacccggagacgctggtcaacctcatcgtcctgtcccagcacctgggcaagccccctgaggtgacaaaccgatacctgtcccagctgaaggatgcccacaggtcccatcccttcatcaaggagtaccaggccaaggagaacgactttgacaggctggtgctacagtacgctcccagcgcctgaggctggcccagagctgtcaggaccatgaagccaggacagaggccaggagccagccctgcagccctccccacccggcatccacctgcatcccctctgggggcaggagcccacccccagcacccccatctgttaataaatatctcaactccagggtgttccacctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - protein, 308 aa, Artificial Sequence
  Description of Artificial Sequence/note = synthetic construct
MAPPAPGPASGGSGEVDELFDVKNAFYIGSYQQCINEAQRVKLSSPERDVERDVFLYRAYLAQRKFGVVLDEIKPSSAPELQAVRMFADYLAHESRRDSIVAELDREMSRSVDVTNTTFLLMAASIYLHDQNPDAALRALHQGDSLECTAMTVQILLKLDRLDLARKELKRMQDLDEDATLTQLATAWVSLATGGEKLQDAYYIFQEMADKCSPTLLLLNGQAACHMAQGRWEAAEGLLQEALDKDSGYPETLVNLIVLSQHLGKPPEVTNRYLSQLKDAHRSHPFIKEYQAKENDFDRLVLQYAPSA

SEQ ID NO: 19 - DNA, 3528 bp, Artificial Sequence
  Description of Artificial Sequence/note = synthetic construct
agccaaggactctggagccgccgccgccgctgctgcggttcatatccggagtagacggagccgcagtagacggatccgcggctgcaccaaaccactgcccctcggagcctggtagtgggccacaagcccccagtcccagaggcgtggtgggtcgggcagagtcggaagaactggctttctagctggaagatgcggaaggggagcgactaggccgcttgcgtctgggcctggcagaagggaccggattttctggcatccttaaatcttgtgtcaaggattggttataatataaccagaaaccatgacggcggctgagaacgtatgctacacgttaattaacgtgccaatggattcagaaccaccatctgaaattagcttaaaaaatgatctagaaaaaggagatgtaaagtcaaagactgaagctttgaagaaagtaatcattatgattctgaatggtgaaaaacttcctggacttctgatgaccatcattcgttttgtgctacctcttcaggatcacactatcaagaaattacttctggtattttgggaaattgttcctaaaacaactccagatgggagacttttacatgagatgatccttgtatgtgatgcatacagaaaggatcttcaacatcctaatgaatttattcgaggatctactcttcgttttctttgcaaattgaaagaagcagaattgctagaacctttaatgccagctattcgtgcatgtttggagcatcgacacagctatgttagaagaaatgctgttttggccatctataccatctatagaaattttgaacatcttatacctgatgctcctgaactgatacatgattttctggtgaatgagaaggatgcaagttgcaaaaggaatgcatttatgatgctaattcatgcagatcaggatcgagctttggattacttaagtacttgcattgatcaagttcaaacatttggagacattctgcagctggttattgttgaactgatttataaggtctgtcatgctaatccatcagaaagagctcgttttattcgctgcatctataacttattacagtcatccagccctgctgtaaaatatgaagctgctgggacattagtgacactctctagtgcaccaactgcaatcaaggctgctgctcagtgttacattgatttaattattaaggagagcgacaacaatgtaaaactcatagttttggatcgcttgatagaattaaaagagcatcctgctcatgaacgagtactacaggatctggttatggatatcctaagagtattgagcacaccagacttagaagtacgaaagaaaactctgcagttagcactggatcttgtctcttctagaaatgttgaagagctggttattgtcctgaagaaggaagtgataaaaacaaataatgtgtctgagcatgaagatactgacaaatacagacaactcctagtgcgaacattgcattcctgttctgtccgatttccagatatggctgcaaatgttattcctgtgttaatggaatttctcagtgacaacaacgaagcagcagctgctgatgtcttggagtttgttcgtgaagccattcagcgctttgataacctgagaatgcttattgttgagaagatgcttgaagtctttcatgctattaaatctgtcaagatttaccgaggagcattatggatcctgggagaatactgtagtaccaaggaagacattcagagtgtgatgactgagatccgcaggtcccttggagagatcccaattgtagagtcagaaataaagaaagaagctggtgaattaaaacctgaagaagaaataactgtagggccagttcagaaattggttactgaaatgggtacctatgcaactcagagtgcccttagcagttctagacccaccaagaaagaggaagacagacctcccttgagaggattccttctggatggagatttctttgttgctgcctcccttgccacaactctgaccaagattgcattgcgctatgtagctttggttcaggagaagaaaaagcaaaattcttttgttgctgaggctatgttgctcatggctactatcctgcatttgggaaaatcctctcttcctaagaagccaattactgatgatgatgtggatcgaatttccctgtgcctcaaggtcttgtctgaatgttcacctttaatgaatgacattttcaataaggaatgcagacagtccctttctcacatgttatctgctaaactagaagaagagaaattatcccaaaagaaagaatctgaaaagaggaatgtgacagtacagcctgatgaccccatttccttcatgcaactaactgctaagaatgaaatgaactgcaaggaagatcagtttcagctgagtttactggcagcaatgggtaacacacagaggaaagaggcagcagatcccctagcatctaaacttaacaaggtcacccaattgacaggtttctcagatcctgtatatgcagaagcttacgttcatgtcaaccaatatgatattgtcctggatgtacttgttgtgaaccaaaccagtgatactttgcagaattgcacattagaactagctacactaggggatctgaaacttgtggaaaagccgtctcctttgactcttgctcctcatgacttcgcaaatattaaagctaacgtcaaagtagcatcaacagaaaatggaataatttttggtaatatagtttatgatgtctctggagcagcaagtgacagaaattgtgtggttctcagtgatattcacatcgacatcatggactatatccagcctgcaacttgcactgatgcagaattccgtcagatgtgggccgaatttgaatgggaaaacaaagtgacagttaacaccaacatggttgatttaaatgactacttacagcacatattaaagtcaaccaatatgaaatgcctgactccagaaaaggccctttctggttactgtggctttatggcagccaacctttatgctcgttccatatttggtgaagatgcacttgcaaatgtcagcattgagaagccaattcaccagggaccagatgctgctgttaccggccatataagaattcgtgcaaagagccagggaatggccttaagtcttggagataaaatcaacttgtcacagaagaaaactagtatataaaaataaacaaaaagtccttgaagctttacagttaatttaggtatgggcttactggactccaacatcttttgtactctttcatgcttatatagaatctgagttcatgctgaatacttttcagccaataatttatagcctttcccttaaatcaagattgagtttaaaattatagtttgtcttttgtcttaacagttctgaatgctgtcctcaaagtatataatgtttcatgtaccaagacccttttcacagtacaataaacagatctattcataaatttttgttattttataaataaatgattacataattttagttataaaaaaaaaaaaaaaaaaaaagaaaaaaaaaaaaaaaaaaaaaaaaaa

SEQ ID NO: 20 - protein, 953 aa, Artificial Sequence
  Description of Artificial Sequence/note = synthetic construct
MTAAENVCYTLINVPMDSEPPSEISLKNDLEKGDVKSKTEALKKVIIMILNGEKLPGLLMTIIRFVLPLQDHTIKKLLLVFWEIVPKTTPDGRLLHEMILVCDAYRKDLQHPNEFIRGSTLRFLCKLKEAELLEPLMPAIRACLEHRHSYVRRNAVLAIYTIYRNFEHLIPDAPELIHDFLVNEKDASCKRNAFMMLIHADQDRALDYLSTCIDQVQTFGDILQLVIVELIYKVCHANPSERARFIRCIYNLLQSSSPAVKYEAAGTLVTLSSAPTAIKAAAQCYIDLIIKESDNNVKLIVLDRLIELKEHPAHERVLQDLVMDILRVLSTPDLEVRKKTLQLALDLVSSRNVEELVIVLKKEVIKTNNVSEHEDTDKYRQLLVRTLHSCSVRFPDMAANVIPVLMEFLSDNNEAAAADVLEFVREAIQRFDNLRMLIVEKMLEVFHAIKSVKIYRGALWILGEYCSTKEDIQSVMTEIRRSLGEIPIVESEIKKEAGELKPEEEITVGPVQKLVTEMGTYATQSALSSSRPTKKEEDRPPLRGFLLDGDFFVAASLATTLTKIALRYVALVQEKKKQNSFVAEAMLLMATILHLGKSSLPKKPITDDDVDRISLCLKVLSECSPLMNDIFNKECRQSLSHMLSAKLEEEKLSQKKESEKRNVTVQPDDPISFMQLTAKNEMNCKEDQFQLSLLAAMGNTQRKEAADPLASKLNKVTQLTGFSDPVYAEAYVHVNQYDIVLDVLVVNQTSDTLQNCTLELATLGDLKLVEKPSPLTLAPHDFANIKANVKVASTENGIIFGNIVYDVSGAASDRNCVVLSDIHIDIMDYIQPATCTDAEFRQMWAEFEWENKVTVNTNMVDLNDYLQHILKSTNMKCLTPEKALSGYCGFMAANLYARSIFGEDALANVSIEKPIHQGPDAAVTGHIRIRAKSQGMALSLGDKINLSQKKTSI

SEQ ID NO: 21 - DNA, 3075 bp, Artificial Sequence
  Description of Artificial Sequence/note = synthetic construct
ggcactgtagcaccgctactccgtgccgcgcccgtcgagcattgcgttgctgcattgcgccccaccgactccactatgttgaagaaattcgacaagaaggatgaggagtcaggtggaggctccaacccattccagcaccttgagaagagtgcggtactccaggaggcccgtgtatttaatgaaactcccatcaaccctcggaaatgtgcccacatcctcaccaagattctttatctcataaaccagggggagcacctggggaccacggaagcgaccgaggccttctttgccatgaccaagctctttcagtccaatgaccccacactccgtcggatgtgctacttgaccatcaaggagatgtcttgcattgcagaggatgtcatcattgtcaccagcagcctaacaaaagacatgactgggaaagaagacaactaccggggcccggccgtgcgagccctctgccagatcactgatagcaccatgctgcaggctattgagcgctacatgaaacaagccattgtggacaaggtgcccagtgtctccagctctgccctcgtgtcttccttgcacctgctgaagtgcagctttgacgtggtcaagcgctgggtgaatgaggctcaggaggcagcatccagtgataacatcatggtccagtaccacgcactagggctcctgtaccatgtgcgtaagaatgaccgcctagccgtcaataagatgatcagcaaggtcacacggcatggccttaagtctccctttgcctactgcatgatgatccgggtggccagcaagcagctggaagaggaggatggcagccgtgacagcccactgtttgacttcatcgagagctgcttgcgcaacaagcacgagatggtggtgtatgaagccgcctcggccattgtcaacctgcctgggtgcagcgccaaggagctggccccagctgtctcagtgctccagctcttctgcagctcccccaaggccgccctccgttacgccgccgtccgcaccctcaacaaggtggccatgaagcacccgtccgctgtgacagcttgtaatctggatctggagaacctggtcacagattcaaaccgcagcattgccacgctggccatcaccaccctccttaagacgggcagcgagagcagcatcgaccgcctcatgaagcagatctcctccttcatgtcagaaatctcggatgaattcaaggtggtggttgtccaggccatcagtgccctgtgtcagaaatatcctcgcaaacacgccgtccttatgaacttcctgttcaccatgctgcgggaagagggtggctttgagtataagcgcgctatcgtggactgcatcatcagcatcattgaagagaactcagagagcaaggagacagggctgtcacatctgtgcgagttcatcgaggactgcgagttcacagtgctggccacccgtattctacatctcctgggccaggaggggcccaagaccaccaatccctcaaagtacatccgcttcatctataaccgagtggtcttggagcatgaggaggtccgggcaggtgctgtgagtgctctggcgaagtttggagcccagaatgaagagatgttacccagtatcttggtgttgctgaagaggtgtgtgatggatgatgacaatgaagtaagggaccgagccaccttctacctaaatgtcctggagcagaagcagaaggcccttaatgcaggctatatcctaaatggtctgactgtgtccatccctggtctggagagggctctgcagcagtacactctagaaccatcagaaaaaccttttgacctcaagtctgtgcccctggccacggcgcccatggcagagcagagaacagaaagtacccccatcacagcagtcaaacagcctgagaaagtggcagctaccaggcaggagatcttccaggagcagttggcagcagtgccagagttccgcggtcttgggcccctcttcaagtcctcgcctgagcccgtggccctcaccgagtcagagacggagtatgtcatccgctgcaccaaacacaccttcaccaaccacatggtttttcagtttgactgcacaaacacactcaatgaccagaccttggagaatgtcacagtgcagatggagcccactgaggcctatgaggtgctctgttacgtgcctgcccggagcctgccctacaaccagcccgggacctgctacacactggtggcactgcccaaagaagaccccacagctgtggcctgcacattcagctgcatgatgaagttcactgtcaaggactgtgatcccaccactggggagactgatgacgaaggctatgaggatgagtatgtgctggaagatctggaagttactgtagctgatcacattcaaaaggtcatgaaactgaacttcgaagcagcctgggatgaggtaggggatgaatttgagaaggaggaaacgttcaccttgtctaccatcaagacacttgaagaggctgtgggtaatattgtgaagttcttgggaatgcacccttgtgagaggtcagacaaagtgccggataacaagaacacccacacgttgctcctggctggtgtgttccggggtggtcatgacatcctggtgcgctcccggctgctgcttttggacacagtgacaatgcaggtgacagccagaagtttggaggagctgccagtagacatcatcttggcatctgtgggataagaggccagcctgcataggacctcatacccttccccaacactacctggaagttgtgccttcctcatgaaactggcagaaaccccttcccaagcttctgtattgaaaaacaattaggaatcattgcagatttttttttattctgctcccacctcccacccgggactacttgctggtgactttttttttttttttttttaaataggggatgattttagcttgtcctaaatcttgctgtccacccttccaggaaagggacattgtaaatgaataaaacattctcaactcctcttgaatctatcccccaagaaaccatcttatccctgtaataaatcagcatgtatttattgaaaaaaaaaaaaaaaaaaaaaaaaaaaa

SEQ ID NO: 22 - protein, 874 aa, Artificial Sequence
  Description of Artificial Sequence/note = synthetic construct
MLKKFDKKDEESGGGSNPFQHLEKSAVLQEARVFNETPINPRKCAHILTKILYLINQGEHLGTTEATEAFFAMTKLFQSNDPTLRRMCYLTIKEMSCIAEDVIIVTSSLTKDMTGKEDNYRGPAVRALCQITDSTMLQAIERYMKQAIVDKVPSVSSSALVSSLHLLKCSFDVVKRWVNEAQEAASSDNIMVQYHALGLLYHVRKNDRLAVNKMISKVTRHGLKSPFAYCMMIRVASKQLEEEDGSRDSPLFDFIESCLRNKHEMVVYEAASAIVNLPGCSAKELAPAVSVLQLFCSSPKAALRYAAVRTLNKVAMKHPSAVTACNLDLENLVTDSNRSIATLAITTLLKTGSESSIDRLMKQISSFMSEISDEFKVVVVQAISALCQKYPRKHAVLMNFLFTMLREEGGFEYKRAIVDCIISIIEENSESKETGLSHLCEFIEDCEFTVLATRILHLLGQEGPKTTNPSKYIRFIYNRVVLEHEEVRAGAVSALAKFGAQNEEMLPSILVLLKRCVMDDDNEVRDRATFYLNVLEQKQKALNAGYILNGLTVSIPGLERALQQYTLEPSEKPFDLKSVPLATAPMAEQRTESTPITAVKQPEKVAATRQEIFQEQLAAVPEFRGLGPLFKSSPEPVALTESETEYVIRCTKHTFTNHMVFQFDCTNTLNDQTLENVTVQMEPTEAYEVLCYVPARSLPYNQPGTCYTLVALPKEDPTAVACTFSCMMKFTVKDCDPTTGETDDEGYEDEYVLEDLEVTVADHIQKVMKLNFEAAWDEVGDEFEKEETFTLSTIKTLEEAVGNIVKFLGMHPCERSDKVPDNKNTHTLLLAGVFRGGHDILVRSRLLLLDTVTMQVTARSLEELPVDIILASVG

SEQ ID NO: 23 - DNA, 3110 bp, Artificial Sequence
  Description of Artificial Sequence/note = synthetic construct
tcgccggctgcggcgcctgggacggttgcggtgggtctgggcgctgggaagtcgtccaagatgattaaaaaattcgacaagaaggacgaggagtctggtagtggctccaatcctttccagcatctggagaagagtgctgttttacaggaggctcgtatattcaatgaaactccaatcaatccaagaagatgtttgcatattcttacaaagattctttacttactgaaccagggtgaacactttggaacaacggaagctacagaagccttctttgcaatgacgcgattgtttcaatctaatgatcaaacattgaggagaatgtgctaccttaccatcaaagaaatggctaccatctctgaggatgtgataattgtcacaagcagtctgactaaagacatgactggaaaagaagatgtataccgaggcccggccatcagagctctctgcaggatcaccgatggaacaatgttgcaagccattgaaagatacatgaagcaggccattgtggataaagtttccagtgtatccagttcagcactggtatcttccctgcacatgatgaagataagctatgatgtggttaagcgctggatcaatgaagcccaagaagctgcatcaagtgataatattatggtccagtaccatgcattgggagtcctgtatcaccttagaaagaatgatcgacttgctgtttccaagatgttgaataagtttactaaatctggtctcaagtcacagtttgcttactgcatgctgatccgaattgccagtcgcttactaaaagaaactgaggatggccatgaaagtccactgtttgatttcattgagagctgcttgcgaaataaacatgaaatggttatttatgaagctgcttcagctatcatccatcttcctaactgcactgcaagagagttggcacctgctgtttcagttcttcaacttttctgtagttctcctaagccagccttgagatatgcagctgtgaggaccttgaacaaggtggcaatgaagcacccctctgctgttactgcctgcaatctggacttagaaaacttaatcacagactcaaacagaagcattgctaccttagccattactacactcctcaaaacaggaagtgagagcagtgtggaccggctcatgaagcagatatcttcttttgtgtctgaaatctcagatgagttcaaggtggtggttgtacaggcaattagtgctctctgtcagaaataccctcgaaagcacagtgtcatgatgactttcctctccaacatgctccgagatgatggaggctttgagtacaagcgggccattgtggactgtataatcagcattgtggaagagaaccctgagagtaaagaagcaggcctagcccacctttgtgaattcattgaggactgtgaacacactgttctggctactaagattctacacttgttgggcaaagagggccctagaacgcctgtcccctccaaatatatccgttttatttttaatagggttgtcctggagaatgaggctgtcagagctgctgctgtgagtgctttggctaaatttggggctcagaatgagagtcttctcccaagcatccttgtactcttacagaggtgtatgatggatactgatgacgaggtacgagacagagctaccttctatctgaatgtgctgcagcagaggcagatggcactaaatgccacatatatctttaatggtttgacggtctctgtaccagggatggaaaaagccttacaccagtacacgttggagccttcagaaaaaccgtttgacatgaaatcaattcctcttgctatggctcctgtctttgaacagaaagcagaaatcacacttgtggctactaagccagagaagttggctccttccaggcaagacattttccaagaacaattggctgccattcctgagtttctgaatataggacccttgttcaagtcttctgagcctgttcaacttacagaagcagagacagaatattttgttcgatgtatcaagcacatgtttaccaatcacatcgtgttccagtttgactgcaccaacactctcaatgaccagctgctggaaaaagtgacagtgcagatggagccatcagattcctatgaagtgctgtcttgtatcccagcccccagccttccttataaccaaccaggaatatgttacactcttgttcgtttgcctgatgatgaccctacagcagttgcaggctcctttagctgcaccatgaagtttacagtccgggactgtgaccctaacactggagttccagatgaggatgggtatgatgatgagtatgtgctggaagatctcgaagtgactgtgtctgaccatattcagaaagtactgaagcctaactttgctgctgcttgggaagaggtgggagatacctttgagaaagaggaaacctttgccctcagttctaccaaaacccttgaagaggctgtcaacaatatcatcacatttctgggcatgcagccatgtgagaggtccgataaagtacctgagaacaagaattcccattcgctctatctggcaggtatattcagaggtggctatgatttattggtgaggtccaggctggccttagccgatggagtgaccatgcaggtgactgtcagaagtaaagagagaacacctgtagatgttatcttagcttctgttggataaatgcttactggacaagaggaaactgatgcacactacatggtcagtgggcttttaggctagtggcatcagtttcccagaatcagacttttgaagatgaatgactttggagaagcaaattaaacatttggccctgagccagcagatcaagcaaatgtctatctttgcgcatgggttgttttttttttttttctttttattctacttggtcagctttgggacgatagtgcagctttgggtgatcttgaaaatcaaatactatcctatactccagctgcttaacttcattttattctttaatgtgtacctgaaagctcctggcaatgctggaaaatttttatcccagaggggtgggggggaggggggaggggaagccagagtccacttttgtcacaattcatttttattaatagaaaataaacacttattccagtttc

SEQ ID NO: 24 - protein, 871 aa, Artificial Sequence
  Description of Artificial Sequence/note = synthetic construct
MIKKFDKKDEESGSGSNPFQHLEKSAVLQEARIFNETPINPRRCLHILTKILYLLNQGEHFGTTEATEAFFAMTRLFQSNDQTLRRMCYLTIKEMATISEDVIIVTSSLTKDMTGKEDVYRGPAIRALCRITDGTMLQAIERYMKQAIVDKVSSVSSSALVSSLHMMKISYDVVKRWINEAQEAASSDNIMVQYHALGVLYHLRKNDRLAVSKMLNKFTKSGLKSQFAYCMLIRIASRLLKETEDGHESPLFDFIESCLRNKHEMVIYEAASAIIHLPNCTARELAPAVSVLQLFCSSPKPALRYAAVRTLNKVAMKHPSAVTACNLDLENLITDSNRSIATLAITTLLKTGSESSVDRLMKQISSFVSEISDEFKVVVVQAISALCQKYPRKHSVMMTFLSNMLRDDGGFEYKRAIVDCIISIVEENPESKEAGLAHLCEFIEDCEHTVLATKILHLLGKEGPRTPVPSKYIRFIFNRVVLENEAVRAAAVSALAKFGAQNESLLPSILVLLQRCMMDTDDEVRDRATFYLNVLQQRQMALNATYIFNGLTVSVPGMEKALHQYTLEPSEKPFDMKSIPLAMAPVFEQKAEITLVATKPEKLAPSRQDIFQEQLAAIPEFLNIGPLFKSSEPVQLTEAETEYFVRCIKHMFTNHIVFQFDCTNTLNDQLLEKVTVQMEPSDSYEVLSCIPAPSLPYNQPGICYTLVRLPDDDPTAVAGSFSCTMKFTVRDCDPNTGVPDEDGYDDEYVLEDLEVTVSDHIQKVLKPNFAAAWEEVGDTFEKEETFALSSTKTLEEAVNNIITFLGMQPCERSDKVPENKNSHSLYLAGIFRGGYDLLVRSRLALADGVTMQVTVRSKERTPVDVILASVG

SEQ ID NO: 25 - DNA, 1900 bp, Artificial Sequence
  Description of Artificial Sequence/note = synthetic construct
ttttgcggctccacgtcggcaccagctgcggggcaagatggaggcgctgattttggaaccttccctgtatactgtcaaagccatcctgattctggacaatgatggagatcgactttttgccaagtactatgacgacacctaccccagtgtcaaggagcaaaaggcctttgagaagaacattttcaacaagacccatcggactgacagtgaaattgccctcttggaaggcctgacagtggtatacaaaagcagtatagatctctatttctatgtgattggcagctcctatgaaaatgagctgatgcttatggctgttctgaactgtctcttcgactcattgagccagatgctgaggaaaaatgtagaaaagcgagcactgctggagaacatggaggggctgttcttggctgtggatgaaattgtagatggaggggtgatcctagagagtgatccccagcaggtggtacaccgggtggcattaaggggtgaagatgtcccccttacggagcagaccgtgtctcaggtgctgcagtcagccaaagaacagatcaagtggtcactccttcggtgaagacctcactgttcctggctcttcatcctcttcaaaaaatttgcatgtctgctgtgaattttcatctagttccccaatcgatgctctcagggtcatctcggggatcacagggatccttaaatctccattctgtttgtggttgccccctcaacctcccctacacccttcctattctttttcattcttcttgcagttctgggagtaaagctcccagcatatttagataatagggcaggggaagcaccctctttctttctagactggattatgctcacatgctcccttgccctgacatttttgtaaattctgtgccctttgctgtagctacacttcagattaaagtaggagaaagaatgtgctgagtgttttcctccctttgcctctacctggccctcatcccaacagcccagcaaggggagagagaaagagaattcttttctatagaacgagtgggggcggggatgggtagggatttatccaatctaagccctaaccccacttagtgacctcagtgttttcttccattccttcttactgccctgtcctctgccttggaagaggctttgggaatagttcatagggaagggacaacatggaagaaacagcgatttaaattgtattgaacagggcatataaaatgcattctgtaccctgatctggcatatagcttcaaaactgcagtggcgagtgtccatctcttagttagctaccttaactgtccacccttactacctgtgggatcgttgcctggtttgtcttctctgtgtcctggagcaaagccagttcctaaaactaaaactccattctagtcttgggaagaaaagtttctactcagaactggggaaggagtggaacttatgacttgggcctctaggctgtctctgtcccctcagctccccgacatgcatttactctctgccgtgggtctgcagtcgctgcaacctaccctctctctgcctcagccttacacccaagcagtaggtctgtgctctccctgtctctaggtcgctgagagaggtgcttttcttcataaaacctttggggtttggatttccccaggaagatggagaatggaatactcactcttgggtctaatctttccccttgacccagaacttcctccccacaaaaatgcctttaaaaaccttcctgagacttaagcattctgccccacttactaactgccagttctccagcactgaggtggggcagataacggggcatatttaagggggcatctttgtgtaaaagatgcatggagtcaggagaaaaccaccttcataaactgctctgtgcaaagaggaataaaacattttttccaaactgaaa

SEQ ID NO: 26 - protein, 177 aa, Artificial Sequence
  Description of Artificial Sequence/note = synthetic construct
MEALILEPSLYTVKAILILDNDGDRLFAKYYDDTYPSVKEQKAFEKNIFNKTHRTDSEIALLEGLTVVYKSSIDLYFYVIGSSYENELMLMAVLNCLFDSLSQMLRKNVEKRALLENMEGLFLAVDEIVDGGVILESDPQQVVHRVALRGEDVPLTEQTVSQVLQSAKEQIKWSLLR

SEQ ID NO: 27 - protein, 19 aa, Artificial Sequence
  Description of Artificial Sequence/note = synthetic construct
CESGELKPEDDVTVGPAQK

SEQ ID NO: 28 - protein, 16 aa, Artificial Sequence
  Description of Artificial Sequence/note = synthetic construct
GNMFANLFKGLFGKKE

SEQ ID NO: 29 - protein, 16 aa, Artificial Sequence
  Description of Artificial Sequence/note = synthetic construct
EKKGFLGKFLNAFMNG

SEQ ID NO: 30 - protein, 13 aa, Artificial Sequence
  Description of Artificial Sequence/note = synthetic construct
CTHPFTHECGGGS

SEQ ID NO: 31 - protein, 13 aa, Artificial Sequence
  Description of Artificial Sequence/note = synthetic construct
CGEHVRPSCGGGS

SEQ ID NO: 32 - protein, 13 aa, Artificial Sequence
  Description of Artificial Sequence/note = synthetic construct
CNNTRSPYCGGGS

SEQ ID NO: 33 - protein, 13 aa, Artificial Sequence
  Description of Artificial Sequence/note = synthetic construct
CEQTLYRVCGGGS

SEQ ID NO: 34 - DNA, 3916 bp, Artificial Sequence
  Description of Artificial Sequence/note = synthetic construct
gcttggggccgccatcttggcaagaggcgaagcggcagcggttcctgtcaagggggcagcaggtccagagctgctggtgctcccgttccccagaccctacccctatccccagtggagccggagtgcgggcgcgccccaccaccgccctcaccatggtgctgttggcagcagcggtctgcacaaaagcaggaaaggctattgtttctcgacagtttgtggaaatgacccgaactcggattgagggcttattagcagcttttccaaagctcatgaacactggaaaacaacatacgtttgttgaaacagagagtgtaagatatgtctaccagcctatggagaaactgtatatggtactgatcactaccaaaaacagcaacattttagaagatttggagaccctaaggctcttctcaagagtgatccctgaatattgccgagccttagaagagaatgaaatatctgagcactgttttgatttgatttttgcttttgatgaaattgtcgcactgggataccgggagaatgttaacttggcacagatcagaaccttcacagaaatggattctcatgaggagaaggtgttcagagccgtcagagagactcaagaacgtgaagctaaggctgagatgcgtcgtaaagcaaaggaattacaacaggcccgaagagatgcagagagacagggcaaaaaagcaccaggatttggcggatttggcagctctgcagtatctggaggcagcacagctgccatgatcacagagaccatcattgaaactgataaaccaaaagtggcacctgcaccagccaggccttcaggccccagcaaggctttaaaacttggagccaaaggaaaggaagtagataactttgtggacaaattaaaatctgaaggtgaaaccatcatgtcctctagtatgggcaagcgtacttctgaagcaaccaaaatgcatgctccacccattaatatggaaagtgtacatatgaagattgaagaaaagataacattaacctgtggacgagacggaggattacagaatatggagttgcatggcatgatcatgcttaggatctcagatgacaagtatggccgaattcgtcttcatgtggaaaatgaagataagaaaggggtgcagctacagacccatccaaatgtggataaaaaacttttcactgcagagtctctaattggcctgaagaatccagagaagtcatttccagtcaacagtgacgtaggggtgctaaagtggagactacaaaccacagaggaatcttttattccactgacaattaattgctggccctcggagagtggaaatggctgtgatgtcaacatagaatatgagctacaagaagataatttagaactgaatgatgtggttatcaccatcccactcccgtctggtgtcggcgcgcctgttatcggtgagatcgatggggagtatcgacatgacagtcgacgaaataccctggagtggtgcctgcctgtgattgatgccaaaaataagagtggcagcctggagtttagcattgctgggcagcccaatgacttcttccctgttcaagtttcctttgtctccaagaaaaattactgtaacatacaggttaccaaagtgacccaggtagatggaaacagccccgtcaggttttccacagagaccactttcctagtggataagtatgaaattctgtaataccaagaagagggagctgaaaaggaaaattttcagattaataaagaagacgccaatgatggctgaagagtttttcccagatttacaagccactggagaccccttttttctgatacaatgcacgattctctgcgcgcaaggaccctcgactcacccccatgtttcagtgtcacagagacattctttgataaggaaatggcacaaacataaagggaaaggctgctaattttctttggcagattgtattggccagcaggaaagcaagctctccagagaatgcccccagttaaatacctcctctacctttacctaagttgctcctttatttttattttattattattattattattattattttttgagatggagtctcactttgtaacccaggctggaatgcaatggcatgatctcagctcactgcaacctccgcctcctgggttcaagcaagtctcctgcctcagcctccgagtagctgggactacaggtgcacgccaccacgcctggctaattttttgtattttagtagagacggggtttcaccgtgttgcccaggctggtcgcgaactcctgagctcaggcaatccgcccacctcagcctcccaaagtgttgggattacaggcatgagccaccatgcccagctgctcctttattttaatccctaaatataatccctaaatatagttatatttcatacttagtttgtttttaaaaagttttctctgtagaaaattttaatcattcataccctttacctttaggtttttctttctatacattcagtcaggcactgggatcatctgtttacaggcattatatttatttggcactcctggaacaagtatatctaacccattcttgatttttggactattcaggtgaactatttgaggggtatggggtctagaagttaaaagatacgcatgtcttctgttcttttcccgtatcaattcattccttcatctctttgccaagttgttttcctttcagggcctgtccttccagtttagaacagtaccatgaatcccacttgtgtcaatattaaagatagctgagaagcacctttcaaatggcacagtccctcttcaagatgtctaaaagaatggttatgtctgtccagttagggatttcacatccacatgtaatcatgtctgctgctgttgctacccaaattttcatttctccacattttgggtacttaagctaaaacgtaatggccacagtctgtaatccattcacattcctcagtttcaccacctccctcttccagactgcactctctgtcatcagtcccctcctttctaacagaaatggggttatgattttgaaggctgtgggttcagggagtctttgccaatcctgttggccctaaactatcaaggaggctccatttcaccatttgattttttgcatttcaggaggcaactgattgtttcgatatgtacatattactcacgtataccccatttccttccagtcagcccaacattttccaccagtctgtccccatctctgaaatccttccttctctttccccctaagtcttttgagtgtcatcatgtactggtggtttctcggttccatctcatccatttccttttcaatggagactacagcgtcagccagctcagccttggcttttaactcaatattccagtccataggggtggttaaaagttgctgcaaggctgcaggcactggcagtgggaagaggcagacgactagatgacttctgcacttttagctggttgaaaagtaccactcccactctgaacatctggccgtccctgcaaagagtgtactgtgcttgaagcagagcactcacacataaatggctgtgtgtggaattgcttgccaaagaagtttctagcctttccctttcccctaactgcatcagggaagaattcttatctctagcttggtttccacatgaggtttttctgagaagggcttgggacaagaagtctgtcatgttagttaagcaggcaagaaatcctactaatccagttttgtttgaaagttgtttgtccgtatgattttttaaaagtcaagtttaatttcaaaaaaccttttttttctgagattacttttggggtaatatttaaaatgagagacattttgtaaccctgtaaaatacatagggaatataacattccagtgtatacaaagaaggcaaattctttaatcaaataaagcgtattataaaatgagaaaaaaaaaaaaaaaa

SEQ ID NO: 35 - protein, 511 aa, Artificial Sequence
  Description of Artificial Sequence/note = synthetic construct
MVLLAAAVCTKAGKAIVSRQFVEMTRTRIEGLLAAFPKLMNTGKQHTFVETESVRYVYQPMEKLYMVLITTKNSNILEDLETLRLFSRVIPEYCRALEENEISEHCFDLIFAFDEIVALGYRENVNLAQIRTFTEMDSHEEKVFRAVRETQEREAKAEMRRKAKELQQARRDAERQGKKAPGFGGFGSSAVSGGSTAAMITETIIETDKPKVAPAPARPSGPSKALKLGAKGKEVDNFVDKLKSEGETIMSSSMGKRTSEATKMHAPPINMESVHMKIEEKITLTCGRDGGLQNMELHGMIMLRISDDKYGRIRLHVENEDKKGVQLQTHPNVDKKLFTAESLIGLKNPEKSFPVNSDVGVLKWRLQTTEESFIPLTINCWPSESGNGCDVNIEYELQEDNLELNDVVITIPLPSGVGAPVIGEIDGEYRHDSRRNTLEWCLPVIDAKNKSGSLEFSIAGQPNDFFPVQVSFVSKKNYCNIQVTKVTQVDGNSPVRFSTETTFLVDKYEIL